(19) United States
(12) Patent Application Publication
(10) Pub. No.: US 2012/0269849 A1
(43) Pub. Date: Oct. 25, 2012

(54) ATTENUATED INFLUENZA VIRUSES AND VACCINES

(76) Inventors: Eckard Wimmer, East Setauket, NY (US); Steve Skiena, Setauket, NY (US); Steffen Mueller, Kings Point, NY (US); Bruce Futcher, Stony Brook, NY (US); Dimitris Papamichail, South Miami, FL (US); John Robert Coleman, Blauvelt, NY (US); Jeronimo Cello, Port Jefferson, NY (US)

(21) Appl. No.: 13/501,045

(22) PCT Filed: Oct. 11, 2010
(86) PCT No.: PCT/US10/52188

§ 371 Date: Jun. 26, 2012

Related U.S. Application Data
(60) Provisional application No. 61/250,456, filed on Oct. 9, 2009.

(51) Int. Cl. A61K 39/12; C12N 7/04; A61P 31/12; C12N 15/54

(52) U.S. Cl. 424/204.1; 536/23.2; 435/236

(57) ABSTRACT

The present provides attenuated influenza viruses comprising a modified viral genome containing a plurality of nucleotide substitutions. The nucleotide substitutions result in the rearrangement of preexisting codons of one or more protein encoding sequences and changes in codon pair bias. Substitutions of non-synonymous and synonymous codons may also be included. The attenuated influenza viruses enable production of improved vaccines and are used to elicit protective immune responses.

### CROSS-REFERENCE TO RELATED APPLICATIONS

This application claims priority to U.S. Application No. 61/250,456, filed Oct. 9, 2009, which is incorporated herein by reference in its entirety. This application is related to International Patent Application PCT/US2008/058952, which is incorporated herein by reference in its entirety.

### COPYRIGHT NOTICE

A portion of the disclosure of this patent document contains material which is subject to copyright protection. The copyright owner has no objection to the facsimile reproduction by anyone of the patent document or patent disclosure as it appears in the Patent and Trademark Office patent file or records, but otherwise reserves all copyright rights whatsoever.

### FIELD OF THE INVENTION

The present provides attenuated influenza viruses comprising a modified viral genome containing a plurality of nucleotide substitutions. The nucleotide substitutions result in the rearrangement of preexisting codons of one or more protein encoding sequences and changes in codon pair bias. Substitutions of non-synonymous and synonymous codons may also be included. The attenuated influenza viruses enable production of improved vaccines and are used to elicit protective immune responses.

### BACKGROUND OF THE INVENTION

Influenza annually kills 250,000 to 500,000 worldwide despite existing live and inactivated vaccines, motivating the search for new, more effective, vaccines that can be rapidly generated and easily produced. Between 1990 and 1999, influenza caused about 35,000 deaths each year in the U.S. These staggering numbers have not changed significantly over the last two decades in spite of enormous efforts in biomedical research (R. Salomon, R. G. Webster, Cell 136, 402 (Feb. 6, 2009).

Influenza viruses are negative stranded, enveloped orthomyxoviruses with eight gene segments (P. Palese, M. L. Shaw, in Field's Virology, D. M. Knipe et al., Eds., Lippincott Williams & Wilkins (LWW), Philadelphia, 2007, vol. 2, pp. 1647-1689). There are three types of influenza viruses: A, B, and C. The antigenicity of the A and B types of influenza viruses, which cause serious disease, is determined by the two glycoproteins hemagglutinin (HA) and neuraminidase (NA). NA is absent from type C viruses. Antigenicity of both types undergoes yearly genetic drift (by point mutations), which is the basis for seasonal epidemics (D. A. Steinhauer, J. J. Skehel, 2002, Annu Rev Genet 36, 305). Swapping of entire gene segments by reassortment between viruses of aquatic birds, swine and humans produces new type A influenza viruses (genetic shift) that may cause devastating pandemics in a world population that is immunologically naive to them. The genetic capacity of influenza viruses for rapid immune escape demands the annual updating of vaccine strains to reflect the most recent changes in the HA and NA genes within the impending seasonal or pandemic strains. Two types of vaccines are currently used in attempts to control influenza: the standard vaccine of chemically inactivated virus and a recently licensed live attenuated influenza vaccine (LAIV) of cold adapted virus (H. F. Maassab, Feb. 11, 1967, Nature 213), delivered as a nasal-spray (“FluMist”) (CDC; http://www.cdc.gov/flu/protect/keyfacts.htm). Either vaccine comes with certain limitations. While cell-mediated responses are increasingly being recognized as a major determinant of anti influenza immunity (G. F. Rimmelzwaan, R. A. Fouchier, A. D. Osterhaus, December 2007, Curr Opin Biotechnol 18, 529), the traditional, killed vaccines act on the principle of inducing predominantly neutralizing antibodies. LAIV, on the other hand, effectively induce both humoral and cellular immunity, but their production is the result of lengthy trial and error experimentation. When an acceptable, attenuated donor genotype is identified, it must be “reused” in every subsequent, annually updated vaccine. After each annual re-vaccination a 4 mounting cellular immunity against the internal, preserved gene products of the donor strain, or preexisting cellular immunity from natural infections, may limit replication of the live vaccine in the host, ultimately reducing its efficacy to induce neutralizing antibodies against the novel HA and NA proteins.

There are three types of influenza viruses: A, B, and C. Influenza A viruses are further classified by subtype on the basis of the two main surface glycoproteins hemagglutinin (HA) and neuraminidase (NA). Influenza A subtypes and B viruses are further classified by strains.

Wild birds are the natural host for all known subtypes of influenza A viruses. Typically, wild birds do not become sick when they are infected with avian influenza A viruses. However, domestic poultry, such as turkeys and chickens, can become very sick and die from avian influenza, and some avian influenza A viruses also can cause serious disease and death in wild birds.

Influenza type A viruses can infect people, birds, pigs, horses, and other animals, but wild birds are the natural hosts for these viruses. Influenza type A viruses are divided into subtypes and named on the basis of two proteins on the surface of the virus: hemagglutinin (HA) and neuraminidase (NA). For example, an “H7N2 virus” designates an influenza A subtype that has an HA 7 protein and an NA 2 protein. Similarly an “H5N1” virus has an HA 5 protein and an NA 1 protein. There are 16 known HA subtypes and 9 known NA subtypes. Many different combinations of HA and NA proteins are possible. Only some influenza A subtypes (i.e., H1N1, H1N2, and H3N2) are currently in general circulation among people. Other subtypes are found most commonly in other animal species. For example, H7N7 and H3N8 viruses cause illness in horses, and H3N8 also has recently been shown to cause illness in dogs.

There remains a need for a systematic approach to generating attenuated live viruses that have practically no possibility of reversion and thus provide a fast, efficient, and safe method of manufacturing a vaccine. The present invention fulfills this need, is broadly applicable to a wide range of influenza viruses and provides an effective approach for producing anti-viral vaccines.

### SUMMARY OF THE INVENTION

The invention provides a systematic, rational approach, termed Synthetic Attenuated Virus Engineering (SAVE), to develop a new, highly effective live attenuated influenza virus vaccine candidate by rearrangement of synonymous codons, resulting in changes in codon pair bias, usually without changing any viral proteins. Attenuation is based on many hundreds of nucleotide changes in different influenza virus genes and offers high genetic stability and a large margin of safety.

In particular, the invention provides influenza viruses for use in vaccines, in which specific influenza virus genes are deoptimized, primarily or solely by rearrangements of preexisting synonymous codons in the genes, accompanied by reductions in codon pair bias (CPB). In one embodiment of the invention, synonymous codons are only rearranged, so that codon pair bias, but not codon bias, is altered. In other embodiments, codon rearrangement may be accompanied by some degree of codon substitution. Not every codon that can be rearranged need be rearranged. Accordingly, the density of deoptimized codon pairs in a coding sequence can be varied to achieve a desired degree of deoptimization of any given coding sequence. The rearrangements and substitutions may result in changes in RNA secondary structure, CpG dinucleotide content, C+G content, translation frameshift sites, translation pause sites, the presence or absence of tissue specific microRNA recognition sequences, or any combination thereof, in the genome.

The large number of mutations introduced into a sequence by codon rearrangement provides for stably attenuated, live vaccines. Also, each influenza virus vaccine can be designed independently of other vaccines. Thus, unlike the currently available live attenuated Influenza vaccine (FluMist®), the technology is independent of any particular “master” donor strain and can be applied rapidly to any emerging influenza virus as a whole. This is significant for dealing with seasonal epidemics and with pandemics, such as the current new A(H1N1) or the feared A(H5N1) pandemics.

The invention provides an attenuated influenza virus genome which comprises two or more nucleic acids with reduced codon pair bias as compared to the parent nucleic acids from which they are derived. The parent nucleic acids can be naturally occurring, or have been genetically manipulated. Each of the nucleic acids encodes a different influenza protein selected from nucleoprotein (NP), a virion protein, and a polymerase protein. The virion proteins include hemagglutinin (HA) and neuraminidase (NA). The polymerase proteins include three RNA polymerase subunits encoded by the P (also known as PA), PB1, and PB2 genes. In certain embodiments, deoptimization of PB1 creates one or stop codons in the PB1-F2 open reading frame. When the codon pair bias of two nucleic acids is reduced, the nucleic acid pairs are (NP, NA), (NP, P), (NP, PB1), (NP, PB2), (NA, P), (NA, PB1), (NA, PB2), (HA, P), (HA, PB1), (HA, PB2), (P, PB1), (P, PB2), or (PB1, PB2). In an embodiment of the invention, only the codon pair bias of the HA nucleic acid is reduced. In another embodiment of the attenuated virus genome, the codon pair bias of HA is reduced together with the codon pair bias of a second influenza nucleic acid other than NP.

In certain embodiments, the attenuated influenza virus genome comprises three nucleic acids with reduced codon pair bias. Such combinations of deoptimized genes include, but are not limited to: (NP, HA, PB1), (NP, NA, PB1), (NP, HA, NA), (NP, HA, PB2), (NP, NA, PB2), (NP, HA, P), (NP, NA, P), (NP, PB1, PB2), (HA, NA, P), (HA, NA, PB1), and (HA, NA, PB2). In one embodiment, one nucleic acid is NP, the second nucleic acid encodes a virion protein, and the third nucleic acid encodes a polymerase protein.

As mentioned, the parent nucleic acid can be from a naturally occurring virus isolate, or have been genetically manipulated. In one embodiment, the nucleic acids of the attenuated influenza virus genome encoding the nucleoprotein (NP), hemagglutinin (HA), and PB1 polymerase proteins are obtained by shuffling the synonymous codons of the parent nucleic acid. In another embodiment, one or more of the codons of the parent nucleic acid is substituted with a non-synonymous codon prior to or after shuffling. In another embodiment, one or more of the codons of the parent nucleic acid is substituted with a synonymous codon prior to or after shuffling.

According to the invention, an attenuated influenza virus genome is provided wherein the codon pair bias of one or more of the nucleic acids, for example, encoding nucleoprotein (NP), hemagglutinin (HA), and the PB1 polymerase protein, is at least 0.05 less than the codon pair bias of the parent nucleic acid. In another embodiment, the codon pair bias of one of more of the nucleic acids is at least 0.1, or at least 0.2, or at least 0.3, or at least 0.4 less that the codon pair bias of the parent nucleic acid.

The codon pair bias of the nucleic acids of the attenuated influenza virus genome can also be stated in absolute terms. Thus, in an embodiment of the invention, the codon pair bias of one or more of the nucleic acids encoding, for example, nucleoprotein (NP), hemagglutinin (HA), and the PB1 polymerase protein is less than −0.5, or less than −0.1, or less that −0.2, or less that −0.3, or less that −0.4. In an embodiment of the invention, the codon pair bias of the nucleic acids encoding nucleoprotein (NP), hemagglutinin (HA), and the PB1 polymerase protein are all less than −0.5, or less than −0.1, or less that −0.2, or less that −0.3, or less that −0.4.

In another embodiment, the invention provides an attenuated influenza virus which comprises an attenuated influenza virus genome as set forth above. In an embodiment of the invention, the attenuated influenza virus is capable of infecting a human. In another embodiment, the attenuated influenza virus is capable of infecting a bird. In yet another embodiment, the attenuated influenza virus is capable of infecting a pig.

In an embodiment of the invention, a vaccine composition is provided for inducing a protective immune response in a subject, wherein the vaccine composition comprises attenuated viruses, each virus containing two or more deoptimized nucleic acids encoding different influenza proteins selected from nucleoprotein (NP), a virion protein, and a polymerase protein. In one such embodiment, the virion protein is hemagglutinin (HA), and the polymerase protein is PB1. Other combinations of influenza nucleic acids that can be deoptimized are set forth above. In certain embodiments, the codon pair bias of each of the deoptimized nucleic acids is less than the codon pair bias of a parent nucleic acid from which it is derived (i.e., codon pair bias is reduced). Thus, in one embodiment, the nucleic acids encoding nucleoprotein (NP), hemagglutinin (HA), and the PB1 polymerase protein in the vaccine composition all have codon pair biases less than the codon pair bias of the parent nucleic acids from which they are derived. The vaccines can be produced with high titers, and exhibit a large margin of safety (i.e., the difference between LD₅₀ and PD₅₀).

The invention provides a method of eliciting a protective immune response in a subject comprising administering to the subject a prophylactically or therapeutically effective dose of a vaccine composition set forth above. In an embodiment of the invention, the vaccine composition further comprises at least one adjuvant.

### BRIEF DESCRIPTION OF THE FIGURES

FIG. 1 depicts plaque phenotypes and growth kinetics of codon-pair deoptimized influenza viruses. (A) Plaque phenotypes on MDCK cells of PR8 wildtype virus and synthetic PR8 derivatives, carrying one (NP^{Min}, HA^{Min}, PB1^{Min}), two (NP/HA^{Min}; HA/PB1^{Min}) or three (PR8^{3F}) deoptimized gene segments. (B) Growth kinetics of PR8 wildtype virus and synthetic PR8 derivatives in MDCK cells after infection with 0.001 MOI of the indicated viruses.

FIG. 2 depicts attenuation of deoptimized Influenza virus PR8^{3F} in BALB/c mice. (A) Body weight curve following intranasal infection with 10⁴ PFU of PR8 wildtype (triangles), 10⁴ PFU of deoptimized PR8^{3F} (diamonds), or mock infected (saline; squares). The average of 5 mice per time point and standard deviations are indicated. Wildtype infected mice did not survive beyond day 5 (indicated by a cross). (B) Virus titer in whole lung homogenate after infection with either 10³ PFU PR8 wildtype (squares), or deoptimized PR8^{3F} (circles). Average of three mice per time point. * On day 9 post infection, PR8^{3F} was no longer detectable (below 40 PFU/lung)

FIG. 3 shows immune responses and Vaccine Margin of Safety for wt PR8 and deoptimized PR8^{3F} viruses. The left ordinate indicates the percentage of animals surviving the primary inoculation with (A) PR8^{3F} (black squares) or (B) wt PR8 (black diamonds), at doses ranging between 10⁰ to 10⁶ PFU. After 28 days, the surviving, vaccinated animals were challenged with a single 1000×LD₅₀ of PR8 wildtype virus. Disease and survival were monitored (right ordinate) for PR8^{3F}—(white circles) and PR8—(white triangles) vaccinated mice. (C) 28 days after a primary infection, serum was collected, and anti-influenza serum antibody titers were determined from animals that had received a primary inoculation of 0.01×LD₅₀ (black diamonds) or 0.001×LD₅₀ of PR8^{3F} (black circles), 0.01×LD₅₀ of PR8 (white squares), or saline (black triangles). ELISA antibody titer against PR8 virus antigen is expressed as the lowest reciprocal serum dilution that resulted in a positive ELISA signal (5 standard deviations above background).

FIG. 4 depicts the codon pair bias (CPB) of selected Influenza A/PR8/3/34 genes and their deoptimized counterparts in relationship to the human ORFeome. CPB is expressed as the average codon pair score per codon pair of a given gene, as described in Coleman et al, 2008. Positive and negative CPB signifies the predominance of statistically over- or under-represented codon-pairs, respectively, in an open reading frame. Circles indicate the CPB for each of 14795 human open reading frames, representing the majority of the known, annotated human genes. The CPB of the targeted gene regions in wildtype Influenza HA, NP, and PB1 are within the range of the human gene pool. Following codon-pair deoptimization, the resulting synthetic gene segments (HA^{Min}, NP^{Min}, and PB1^{Min}) are characterized by an extremely negative CPB that is unlike that of any other human gene.

FIG. 5 shows survival following immunization. Five or more BALB/c mice (as indicated), were inoculated once intranasally on Day 0 with deoptomized PR8^{3F} virus at doses ranging from 10⁰ to 10⁶ PFU. Survival was monitored. On Day 28 after the first inoculation, animals were challenged with 1000×LD₅₀ of the PR8 wt virus. Immune protection is confirmed by disease-free survival after lethal challenge with the wildtype virus. At doses of 10³, 10⁴, and 10⁵ PFU, PR8^{3F} was completely safe and protective, thus all the symbols are superimposed at the 100% level.

### DETAILED DESCRIPTION OF THE INVENTION

The present invention relates to the production of attenuated influenza viruses that can be used as vaccines to protect against viral infection and disease. Accordingly, the invention provides an attenuated virus, which comprises a modified viral genome containing nucleotide substitutions engineered in multiple locations in the genome, wherein the substitutions introduce a plurality of rearranged synonymous codons into the genome. In one embodiment, the order of existing codons is changed, as compared to a wild type sequence, while maintaining the wild type amino acid sequence. The change in codon order alters usage of codon pairs, and consequently, reduces codon pair bias. In other embodiments, codon rearrangement and reduced codon pair bias may be accompanied by other sequence changes, including substitution of synonymous codons which leave the encoded amino acid sequence unchanged, or codon substitutions that result in amino acid substitutions. According to the invention, codon pair bias, which is a measure of codon pair usage, can be evaluated for a coding sequence, whether or not codon substitutions are made.

Most amino acids are encoded by more than one codon. See the genetic code in Table 1. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy. To replace a given codon in a nucleic acid by a synonymous but less frequently used codon is to substitute a “deoptimized” codon into the nucleic acid.

TABLE 1
Genetic Code^{a}

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a}The first nucleotide in each codon encoding a particular amino acid is shown in the left-most column; the second nucleotide is shown in the top row; and the third nucleotide is shown in the right-most column.

Codon Bias

As used herein, a “rare” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40% of the time, but use the synonymous CTA only 7% of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens (source: http://www.
kazusa.or.jp/codon/)

| Amino Acid | Codon | Number | /1000 | Fraction |
|---|---|---|---|---|
| Gly | GGG | 636457.00 | 16.45 | 0.25 |
| Gly | GGA | 637120.00 | 16.47 | 0.25 |
| Gly | GGT | 416131.00 | 10.76 | 0.16 |
| Gly | GGC | 862557.00 | 22.29 | 0.34 |
| Glu | GAG | 1532589.00 | 39.61 | 0.58 |
| Glu | GAA | 1116000.00 | 28.84 | 0.42 |
| Asp | GAT | 842504.00 | 21.78 | 0.46 |
| Asp | GAC | 973377.00 | 25.16 | 0.54 |
| Val | GTG | 1091853.00 | 28.22 | 0.46 |
| Val | GTA | 273515.00 | 7.07 | 0.12 |
| Val | GTT | 426252.00 | 11.02 | 0.18 |
| Val | GTC | 562086.00 | 14.53 | 0.24 |
| Ala | GCG | 286975.00 | 7.42 | 0.11 |
| Ala | GCA | 614754.00 | 15.89 | 0.23 |
| Ala | GCT | 715079.00 | 18.48 | 0.27 |
| Ala | GCC | 1079491.00 | 27.90 | 0.40 |
| Arg | AGG | 461676.00 | 11.93 | 0.21 |
| Arg | AGA | 466435.00 | 12.06 | 0.21 |
| Ser | AGT | 469641.00 | 12.14 | 0.15 |
| Ser | AGC | 753597.00 | 19.48 | 0.24 |
| Lys | AAG | 1236148.00 | 31.95 | 0.57 |
| Lys | AAA | 940312.00 | 24.30 | 0.43 |
| Asn | AAT | 653566.00 | 16.89 | 0.47 |
| Asn | AAC | 739007.00 | 19.10 | 0.53 |
| Met | ATG | 853648.00 | 22.06 | 1.00 |
| Ile | ATA | 288118.00 | 7.45 | 0.17 |
| Ile | ATT | 615699.00 | 15.91 | 0.36 |
| Ile | ATC | 808306.00 | 20.89 | 0.47 |
| Thr | ACG | 234532.00 | 6.06 | 0.11 |
| Thr | ACA | 580580.00 | 15.01 | 0.28 |
| Thr | ACT | 506277.00 | 13.09 | 0.25 |
| Thr | ACC | 732313.00 | 18.93 | 0.36 |
| Trp | TGG | 510256.00 | 13.19 | 1.00 |
| End | TGA | 59528.00 | 1.54 | 0.47 |
| Cys | TGT | 407020.00 | 10.52 | 0.45 |
| Cys | TGC | 487907.00 | 12.61 | 0.55 |
| End | TAG | 30104.00 | 0.78 | 0.24 |
| End | TAA | 38222.00 | 0.99 | 0.30 |
| Tyr | TAT | 470083.00 | 12.15 | 0.44 |
| Tyr | TAC | 592163.00 | 15.30 | 0.56 |
| Leu | TTG | 498920.00 | 12.89 | 0.13 |
| Leu | TTA | 294684.00 | 7.62 | 0.08 |
| Phe | TTT | 676381.00 | 17.48 | 0.46 |
| Phe | TTC | 789374.00 | 20.40 | 0.54 |
| Ser | TCG | 171428.00 | 4.43 | 0.05 |
| Ser | TCA | 471469.00 | 12.19 | 0.15 |
| Ser | TCT | 585967.00 | 15.14 | 0.19 |
| Ser | TCC | 684663.00 | 17.70 | 0.22 |
| Arg | CGG | 443753.00 | 11.47 | 0.20 |
| Arg | CGA | 239573.00 | 6.19 | 0.11 |
| Arg | CGT | 176691.00 | 4.57 | 0.08 |
| Arg | CGC | 405748.00 | 10.49 | 0.18 |
| Gln | CAG | 1323614.00 | 34.21 | 0.74 |
| Gln | CAA | 473648.00 | 12.24 | 0.26 |
| His | CAT | 419726.00 | 10.85 | 0.42 |
| His | CAC | 583620.00 | 15.08 | 0.58 |
| Leu | CTG | 1539118.00 | 39.78 | 0.40 |
| Leu | CTA | 276799.00 | 7.15 | 0.07 |
| Leu | CTT | 508151.00 | 13.13 | 0.13 |
| Leu | CTC | 759527.00 | 19.63 | 0.20 |
| Pro | CCG | 268884.00 | 6.95 | 0.11 |
| Pro | CCA | 653281.00 | 16.88 | 0.28 |
| Pro | CCT | 676401.00 | 17.48 | 0.29 |
| Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both. The PV capsid protein is very actively translated, and has a high codon bias.

Codon Pair Bias

In addition, a given organism has a preference for the nearest codon neighbor of a given codon A, referred to a bias in codon pair utilization. A change of codon pair bias, without changing the existing codons, can influence the rate of protein synthesis and production of a protein.

Codon pair bias may be illustrated by considering the amino acid pair Ala-Glu, which can be encoded by 8 different codon pairs. If no factors other than the frequency of each individual codon (as shown in Table 2) are responsible for the frequency of the codon pair, the expected frequency of each of the 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 2). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes the frequencies of codon usage were re-calculated by dividing the number of occurrences of a codon by the number of all synonymous codons coding for the same amino acid. As expected the frequencies correlated closely with previously published ones such as the ones given in Table 2. Slight frequency variations are possibly due to an oversampling effect in the data provided by the codon usage database at Kazusa DNA Research Institute (http://www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 3 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.97 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of 14,795 human genes, are provided herewith as Supplemental Table 1.

TABLE 3
Codon Pair Scores Exemplified by the Amino Acid
Pair Ala-Glu

| amino acid | | expected | observed | obs/exp |
|---|---|---|---|---|
| pair | codon pair | frequency | frequency | ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

As discussed more fully below, codon pair bias takes into account the score for each codon pair in a coding sequence averaged over the entire length of the coding sequence. According to the invention, codon pair bias is determined by

$$CPB = {\sum }_{i = 1}^{k} \frac{CPSi}{k - 1} .$$

Accordingly, similar codon pair bias for a coding sequence can be obtained, for example, by minimized codon pair scores over a subsequence or moderately diminished codon pair scores over the full length of the coding sequence.

Calculation of Codon Air Bias

Every individual codon pair of the possible 3721 non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurances over the number that would have been expected in this set of genes (in this example the human genome). Determining the actual number of occurrences of a particular codon pair (or in other words the likelyhood of a particular amino acid pair being encoded by a particular codon pair) is simply a matter of counting the actual number of occurances of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

To perform these calculations within the human context, the most recent Consensus CDS (CCDS) database of consistently annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair.

$$S ( {P}_{ij} ) = ln ( \frac{{N}_{O} ( {P}_{ij} )}{{N}_{E} ( {P}_{ij} )} ) = ln ( \frac{{N}_{O} ( {P}_{ij} )}{F ( {C}_{i} ) F ( {C}_{j} ) {N}_{O} ( {X}_{ij} )} )$$

In the calculation, is a codon pair occurring with a frequency of N_{O}(Pᵢⱼ) in its synonymous group. Cᵢ and Cⱼ are the two codons comprising Pᵢⱼ, occuring with frequencies F(Cᵢ) and F(Cⱼ) in their synonymous groups respectively. More explicitly, F(Cᵢ) is the frequency that corresponding amino acid Xᵢ is coded by codon Cᵢ throughout all coding regions and F(Cᵢ)=N_{O}(Cᵢ)/N_{O}(Xᵢ), where N_{O}(Cᵢ) and N_{O}(Xᵢ) are the observed number of occurrences of codon Cᵢ and amino acid Xᵢ respectively. F(Cⱼ) is calculated accordingly. Further, N_{O}(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency N_{O}(Pᵢⱼ) over the expected number of occurrences of Nₑ(Pᵢⱼ).

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions (FIG. 7).

The “combined” codon pair bias of an individual coding sequence was calculated by averaging all codon pair scores according to the following formula:

$$S ( {P}_{ij} ) = {\sum }_{l = 1}^{k} \frac{S ( Pij ) l}{k - 1} .$$

The codon pair bias of an entire coding region is thus calculated by adding all of the individual codon pair scores comprising the region and dividing this sum by the length of the coding sequence.

Calculation of Codon Pair Bias, Implementation of Algorithm to Produce Codon Pair Deoptimized Sequences

An algorithm was developed to quantify codon pair bias. Every possible individual codon pair was given a “codon pair score”, or “CPS”. CPS is defined as the natural log of the ratio of the observed over the expected number of occurrences of each codon pair over all human coding regions.

$$CPS = ln ( \frac{F ( AB ) o}{\frac{F ( A ) \times F ( B )}{F ( X ) \times F ( Y )} \times F ( XY )} )$$

Although the calculation of the observed occurrences of a particular codon pair is straightforward (the actual count within the gene set), the expected number of occurrences of a codon pair requires additional calculation. We calculate This expected number is calculated to be independent both of amino acid frequency and of codon bias, similar to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome

Using these calculated CPSs, any coding region can then be rated as using over- or under-represented codon pairs by taking the average of the codon pair scores, thus giving a Codon Pair Bias (CPB) for the entire gene.

$$CPB = {\sum }_{i = 1}^{k} \frac{CPSi}{k - 1}$$

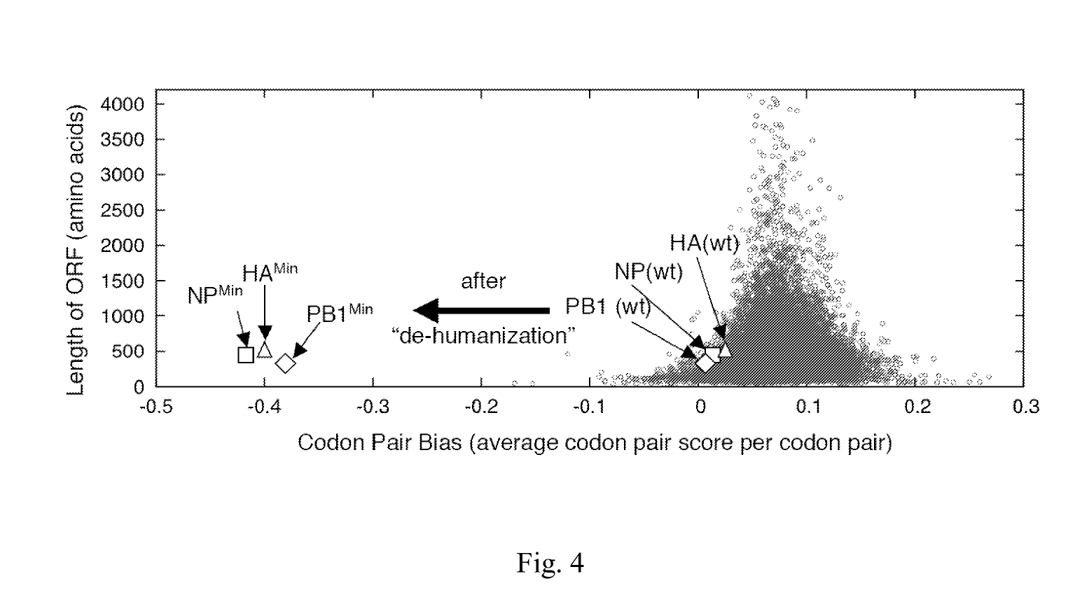

The CPB has been calculated for all annotated human genes using the equations shown and plotted (FIG. 4). Each point in the graph corresponds to the CPB of a single human gene. The peak of the distribution has a positive codon pair bias of 0.07, which is the mean score for all annotated human genes. Also there are very few genes with a negative codon pair bias. Equations established to define and calculate CPB were then used to manipulate this bias.

Algorithm to Produce Codon Pair Deoptimized Sequences

Sequence deoptimization may be performed with or without the aid of a computer, using, for example, a gradient descent, or simulated annealing, or other minimization routine. An example of the procedure that rearranges codons present in a starting sequence can be represented by the following steps:

1) Obtain wildtype viral genome sequence.

2) Select protein coding sequences to target for attenuated design.

3) Lock down known or conjectured DNA segments with non-coding functions.

4) Select desired codon distribution for remaining amino acids in redesigned proteins.

5) Perform random shuffle of at least two synonymous unlocked codon positions and calculate codon-pair score.

6) Further reduce (or increase) codon-pair score optionally employing a simulated annealing procedure.

7) Inspect resulting design for excessive secondary structure and unwanted restriction site:
if yes->go to step (5) or correct the design by replacing problematic regions with wildtype sequences and go to step (8).
8. Synthesize DNA sequence corresponding to virus design.
9. Create viral construct and assess viral phenotype:
if too attenuated, prepare subclone construct and goto 9;
if insufficiently attenuated, goto 2.

Using the formulas above, a computer based algorithm was developed to manipulate the CPB of any coding region while maintaining the original amino acid sequence. The algorithm has the critical ability to maintain the codon usage of a gene (i.e. preserve the frequency of use of each existing codon) but “shuffle” the existing codons so that the CPB can be increased or decreased. The algorithm uses simulated annealing, a mathematical process suitable for full-length optimization (Park, S. et al., 2004). Other parameters are also under the control of this algorithm; for instance, the free energy of the folding of the RNA. This free energy is maintained within a narrow range, to prevent large changes in secondary structure as a consequence of codon re-arrangement. The optimization process specifically excludes the creation of any regions with large secondary structures, such as hairpins or stem loops, which could otherwise arise in the customized RNA. Using this computer software the user simply needs to input the cDNA sequence of a given gene and the CPB of the gene can be customized as the experimenter sees fit.

Source code (PERL script) of a computer based simulated annealing routine is provided.

LENGTHY TABLES
The patent application contains a lengthy table section. A copy of the table is available in electronic form from the USPTO web site (http://seqdata.uspto.gov/?pageRequest=docDetail&DocID=US20120269849A1). An electronic copy of the table will also be available from the USPTO upon request and payment of the fee set forth in 37 CFR 1.19(b)(3).

Alternatively, one can devise a procedure which allows each pair of amino acids to be deoptimized by choosing a codon pair without a requirement that the codons be swapped out from elsewhere in the protein encoding sequence.

Attenuated Influenza Viruses

According to the invention, viral attenuation is accomplished by changes in codon pair bias. While codon bias may also be changed, adjusting codon pair bias is particularly advantageous. For example, attenuating a virus through codon bias generally requires elimination of common codons, and so the complexity of the nucleotide sequence is reduced. In contrast, codon pair bias reduction or minimization can be accomplished while maintaining far greater sequence diversity, and consequently greater control over nucleic acid secondary structure, annealing temperature, and other physical and biochemical properties. The work disclosed herein includes attenuated codon pair bias-reduced or -minimized sequences in which codons are shuffled, but the codon usage profile is unchanged.

Viral attenuation and induction or protective immune responses can be confirmed in ways that are well known to one of ordinary skill in the art, including but not limited to, the methods and assays disclosed herein. Non-limiting examples induce plaque assays, growth measurements, reduced lethality in test animals, and protection against subsequent infection with a wild type virus.

The method is useful for production of influenza virus vaccines, including pandemic and seasonal flu varieties. Such flu varieties include viruses bearing all possible HA-NA combinations. Currently, there are 16 recognized hemagglutinins and nine neuraminidases, each of which has mutational variants. Examples of type A subtypes include, but are not limited to, H10N7, H10N1, H10N2, H10N3, H10N4, H10N5, H10N6, H10N7, H10N8, H10N9, H11N1, H11N2, H11N3, H11N4, H11N6, H11N8, H11N9, H12N1, H12N2, H12N4, H12N5, H12N6, H12N8, H12N9, H13N2, H13N3, H13N6, H13N9, H14N5, H14N6, H15N2, H15N8, H15N9, H16N3, H1N1, H1N2, H1N3, H1N5, H1N6, H1N8, H1N9, H2N1, H2N2, H2N3, H2N4, H2N5, H2N6, H2N7, H2N8, H2N9, H3N1, H3N2, H3N3, H3N4, H3N5, H3N6, H3N8, H3N9, H4N1, H4N2, H4N3, H4N4, H4N5, H4N6, H4N7, H4N8, H4N9, H5N1, H5N2, H5N3, H5N4, H5N6, H5N7, H5N8, H5N9, H6N1, H6N2, H6N3, H6N4, H6N5, H6N6, H6N7, H6N8, H6N9, H7N1, H7N2, H7N3, H7N4, H7N5, H7N7, H7N8, H7N9, H8N2, H8N4, H8N5, H9N1, H9N2, H9N3, H9N4, H9N5, H9N6, H9N7, H9N8, H9N9. Some subtypes of interest include, but are not limited to, H1N1 (one variant of which caused Spanish flu in 1918, another of which is pandemic in 2009), H2N2 (a variant of which caused Asian Flu in 1957), H3N2 (a variant of which caused Hong Kong Flu in 1968, H5N1 (a current pandemic threat), H7N7 (which has unusual zoonotic potential), and H1N2 (endemic in humans and pigs). Examples of attenuated influenza protein coding sequences are provided below.

In the recoded influenza viruses described here, attenuation is the result of numerous nucleotide changes, typically hundreds or thousands, usually without the change of a single amino acid. The attenuated phenotype results from large-scale rearrangements of existing synonymous codons. In contrast, in vaccines in current use, attenuation results from specific mutations that re common to most vaccine strains. Whereas attenuated viruses of the invention express all of the antigenic sites characteristic of the wild type virus from which they are derived, in attenuated vaccines in current use, many of the viral antigens do not correspond to the wild type circulating virus against which immunity is sought. This is because attenuation derives from repeated use of an attenuated “master” donor virus, which is reassorted with heterologous HA and NA genes of the circulating seasonal virus. For this reason, current attenuated vaccines, used repeatedly in seasonal epidemics, may slowly induce cellular immunity to non-virion proteins common to many of the vaccines. Such cellular immunity to non-virion proteins of the master donor virus renders subsequently administered vaccines less capable of inducing a protective immune response against new HA and NA variants. This may limit the usefulness of the only currently licensed LAIV, which is based on cold-adapted influenza strains (H. F. Maassab, 1967), and could explain why current vaccines work better in immunologically naive young children (R. B. Belshe, L. P. Van Voris, J. Bartram, F. K. Crookshanks, December 1984, J. Infect. Dis. 150, 834; R. B. Belshe et al., May 14, 1998, N. Engl. J. Med. 338, 1405) than in adults or the elderly. In fact, in a retrospective review of medical files of over 1 million army personnel, Wang et al. found no significant reduction in influenza-like illness in recipients of the live vaccine (Z. Wang, S. Tobler, J. Roayaei, A. Eick, Mar. 4, 2009, JAMA 301, 945). Supporting this conclusion, a booster with an H3N2 6:2 recombinant in the PR8 genetic background did not induce new neutralizing antibodies against H3 or N2 in macaques previously vaccinated with the H1N1 PR8 progenitor strain, carrying identical backbone genes (A. Sexton et al., August 2009, J. Virol. 83, 7619).

Relatively few amino acid changes (between 5 and 11) in the matrix and polymerase genes are responsible for the attenuated phenotype of the cold adapted LAIV (H. Jin et al., Feb. 1, 2003, Virology 306, 18; M. L. Herlocher, A. C. Clavo, H. F. Maassab, June 1996, Virus Res. 42, 11), the basis of which is not well understood, and as few as 5 amino acid changes completely can revert the cold adapted phenotype (Z. Chen, A. Aspelund, G. Kemble, H. Jin, Feb. 20, 2006, Virology 345, 416).

Influenza viruses recoded by the SAVE method overcomes these limitations of the current LAIV by basing the annual vaccine entirely on the strains actually circulating in the population, without the need of a fixed master donor strain. Since attenuation results from several hundreds or even thousands of nucleotide changes and is additive, the probability of reversion to virulence is extremely low. Further, not only is the margin of safety high, vaccines based on changes in codon pair bias can be generated within weeks for any emerging influenza virus once its genome sequence is known.

According to the invention, attenuated influenza viruses are provided that comprise deoptimized nucleic acids encoding two or more different influenza proteins selected from nucleoprotein (NP) a virion protein, and a polymerase protein. Preferably, the attenuated virus comprises deoptimized nucleic acids that encode nucleoprotein (NP) and a virion protein and a polymerase protein. The virion proteins include hemagglutinin (HA) and neuraminidase (NA). The polymerase proteins include three RNA polymerase subunits encoded by the P, PB1, and PB2 genes. Examples of such combinations of deoptimized genes include, but are not limited to (NP, HA, PB1), (NP, NA, PB1), (NP, HA, NA), (NP, HA, PB2), (NP, NA, PB2), (NP, HA, P), (NP, NA, P), (NP, PB1, PB2), (HA, NA, P), (HA, NA, PB1), and (HA, NA, PB2). Even when the CPB of the nucleoprotein-encoding nucleic acid is minimized, reducing the CPB of one or more of the other genes leads to a greater degree of attenuation.

When the codon pair bias of two nucleic acids is reduced, the nucleic acid pairs are (NP, NA), (NP, P), (NP, PB1), (NP, PB2), (NA, P), (NA, PB1), (NA, PB2), (HA, P), (HA, PB1), (HA, PB2), (P, PB1), (P, PB2), or (PB1, PB2). In one embodiment of the invention, only the codon pair bias of the HA nucleic acid is reduced. In another embodiment of the attenuated virus genome, the codon pair bias of HA is reduced together with the codon pair bias of a second influenza nucleic acid other than NP.

Certain influenza genes are known or thought to overlap, and may encode additional gene products. For example, the M gene encodes a matrix protein (M1) and an ion channel (M2). In this regard, in some wild type viruses, but not others, an 87 amino acid protein, designated PB1-F2, is encoded by an alternate reading frame within the PB1 gene. According to some reports, knocking out the PB1-F2 protein has no effect on viral replication, but diminishes virus pathogenicity in certain models. Accordingly, in viruses having the PB1-F2 open reading frame intact, the PB1 gene can be deoptimized such that codon rearrangement in the PB1 reading frame results in creation of stop codons in the PB1-F2 open reading frame.

As demonstrated herein, viruses of the invention display growth characteristics suitable for vaccine production (e.g., the viruses can be grown and sufficient titers achieved). In addition, with regard to their utility in vaccines, the viruses provide significantly improved safety margins (i.e., a large difference between LD₅₀ and PD₅₀). In particular, in influenza viruses comprising a deoptimized nucleoprotein gene, the presence of a second deoptimized gene results in a useful widening of the gap between a lethal viral dose (LD₅₀) and the dose sufficient to elicit a protective immune response.

Thus, attenuated influenza viruses suitable for vaccine use contain deoptimized nucleic acids encoding two or more different influenza proteins selected from nucleoprotein (NP) a virion protein, and a polymerase protein. In one nonlimiting example of a virus for vaccine use, the NP gene and one or more genes encoding a virion protein are deoptimized. In another such virus, the NP gene and one or more genes encoding a polymerase protein are deoptimized. In another example, the NP gene and the HA gene are deoptimized. In another such virus, the NP gene and the NA gene are deoptimized. In another such virus, the NP gene and the PB1 gene are deoptimized. In yet another embodiment, the NP gene, the HA gene, and the PB1 gene are deoptimized. In another embodiment, the NP gene, the HA gene, and the NA gene are deoptimized. Additional embodiments are like those just described, but wherein the virion protein is NA and/or the polymerase subunit protein is P or PB2, for example, wherein the NP gene, the NA gene, and the PB1 gene are deoptimized, or wherein the NP gene segment, the HA gene, and the PB2 gene are deoptimized.

The invention provides useful combinations of deoptimized influenza virus nucleic acids, which are used in attenuated influenza virus genomes, viruses, and vaccines. In preferred embodiments, attenuation is accomplished by providing nucleic acids with reduced codon pair bias. The nucleic acid combinations can also be deoptimized by other methods in addition to or instead of reduced codon pair bias. For example, the nucleic acids can be deoptimized by substituting rare codons for frequent codons (altering codon bias; Table 2). Thus, in certain embodiments, deoptimized influenza viruses may have a first nucleic acid deoptimized primarily or completely by reducing codon pair bias, and a second nucleic acid deoptimized primarily or completely by substituting rarer codons for more frequent codons.

Vaccine Compositions

The present invention provides a vaccine composition for inducing a protective immune response in a subject comprising any of the attenuated viruses described herein and a pharmaceutically acceptable carrier.

It should be understood that an attenuated virus of the invention, where used to elicit a protective immune response in a subject or to prevent a subject from becoming afflicted with a virus-associated disease, is administered to the subject in the form of a composition additionally comprising a pharmaceutically acceptable carrier. Pharmaceutically acceptable carriers are well known to those skilled in the art and include, but are not limited to, one or more of 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments of the instant vaccine composition, the attenuated virus (i) does not substantially alter the synthesis and processing of viral proteins in an infected cell; (ii) produces similar amounts of virions per infected cell as wt virus; and/or (iii) exhibits substantially lower virion-specific infectivity than wt virus. In further embodiments, the attenuated virus induces a substantially similar immune response in a host animal as the corresponding wt virus.

This invention also provides a modified host cell line specially isolated or engineered to be permissive for an attenuated virus that is inviable in a wild type host cell. Since the attenuated virus cannot grow in normal (wild type) host cells, it is absolutely dependent on the specific helper cell line for growth. This provides a very high level of safety for the generation of virus for vaccine production. Various embodiments of the instant modified cell line permit the growth of an attenuated virus, wherein the genome of said cell line has been altered to increase the number of genes encoding rare tRNAs.

In addition, the present invention provides a method for eliciting a protective immune response in a subject comprising administering to the subject a prophylactically or therapeutically effective dose of any of the vaccine compositions described herein. This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art, and is usually on the order of several weeks to months. The present invention is not limited, however, to any particular method, route or frequency of administration.

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems, and CD155tg transgenic mice expressing the human poliovirus receptor CD155. In a preferred embodiment, the subject is a human. Preferred embodiments of birds are domesticated poultry species, including, but not limited to, chickens, turkeys, ducks, and geese.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold. For example, if a subject has a 1% chance of becoming infected with a virus, a two-fold reduction in the likelihood of the subject becoming infected with the virus would result in the subject having a 0.5% chance of becoming infected with the virus. Most preferably, a “prophylactically effective dose” induces in the subject an immune response that completely prevents the subject from becoming infected by the virus or prevents the onset of the disorder in the subject entirely.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and an instructional material for the use thereof. In further embodiments, the attenuated virus may be one or more poliovirus, one or more rhinovirus, one or more influenza virus, etc. More than one virus may be preferred where it is desirable to immunize a host against a number of different isolates of a particular virus. The invention includes other embodiments of kits that are known to those skilled in the art. The instructions can provide any information that is useful for directing the administration of the attenuated viruses.

Throughout this application, various publications, reference texts, textbooks, technical manuals, patents, and patent applications have been referred to. The teachings and disclosures of these publications, patents, patent applications and other documents in their entireties are hereby incorporated by reference into this application to more fully describe the state of the art to which the present invention pertains. However, the citation of a reference herein should not be construed as an acknowledgement that such reference is prior art to the present invention.

It is to be understood and expected that variations in the principles of invention herein disclosed can be made by one skilled in the art and it is intended that such modifications are to be included within the scope of the present invention. The following Examples further illustrate the invention, but should not be construed to limit the scope of the invention in any way. Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994). All references mentioned herein are incorporated in their entirety by reference into this application.

### EXAMPLES

### Example 1

Nucleic Acids with Reduced Codon Pair Biase Encoding nucleoprotein (NP), hemagglutinin (HA), neuraminidase (NA) and the PB1 polymerase protein. Table 4 provides wild type and mutated sequences encoding influenza virus proteins of the invention. All or part of the coding regions of the PB1, HA, NP, and NA genome segments of several significant influenza viruses were redesigned according to the deoptimization computer program previously described (J. R. Coleman et al., Jun. 27, 2008, Science 320, 1784). The deoptimized segments are suitable for use in vaccines of the invention.

TABLE 4
Deoptimized Influenza A Virus Genes

| | WT | | | Deoptimized | | |
|---|---|---|---|---|---|---|
| | Coding Sequence | | | Coding Sequence | | |
| | SEQ ID | | | SEQ ID | Deoptimized | |
| Gene | NO: | CDS | CPB | NO | Codons | CPB |

H10N7 (A/northern shoveler/California/
HKWF392sm/2007)(Avian)

| | | | | | | |
|---|---|---|---|---|---|---|
| PB1 | 1 | 1-2271 | 0.033 | 2 | 1-757 | −0.435 |
| HA | 3 | 1-1683 | 0.018 | 4 | 1-561 | −0.441 |
| NA | 5 | 1-1494 | 0.009 | 6 | 1-498 | −0.449 |
| NP | 7 | 1-1410 | 0.005 | 8 | 1-470 | −0.450 |

H1N1 (A/New York/3568/2009)(Human)

| | | | | | | |
|---|---|---|---|---|---|---|
| PB1 | 9 | 1-2271 | 0.032 | 10 | 1-757 | −0.427 |
| HA | 11 | 1-1698 | 0.043 | 12 | 1-566 | −0.410 |
| NP | 13 | 1-1494 | 0.048 | 14 | 1-498 | −0.436 |
| NA | 15 | 1-1407 | 0.005 | 16 | 1-469 | −0.456 |

H1N2 (A/New York/211/2003)(Human)

| | | | | | | |
|---|---|---|---|---|---|---|
| PB1 | 17 | 1-2271 | 0.028 | 18 | 1-757 | −0.407 |
| HA | 19 | 1-1695 | 0.036 | 20 | 1-565 | −0.421 |
| NP | 21 | 1-1494 | 0.023 | 22 | 1-498 | −0.447 |
| NA | 23 | 1-1407 | 0.034 | 24 | 1-469 | −0.476 |

H2N2 (A/Albany/22/1957)(Human)

| | | | | | | |
|---|---|---|---|---|---|---|
| PB1 | 25 | 1-2271 | 0.024 | 26 | 1-757 | −0.430 |
| HA | 27 | 1-1686 | 0.040 | 28 | 1-562 | −0.422 |
| NP | 29 | 1-1494 | 0.024 | 30 | 1-498 | −0.464 |
| NA | 31 | 1-1407 | 0.008 | 32 | 1-469 | −0.453 |

H3N2 (A/New York/933/2006)(Human)

| | | | | | | |
|---|---|---|---|---|---|---|
| PB1 | 33 | 1-2271 | 0.021 | 34 | 1-757 | −0.414 |
| HA | 35 | 1-1698 | 0.027 | 36 | 1-566 | −0.447 |
| NP | 37 | 1-1494 | 0.020 | 38 | 1-498 | −0.436 |
| NA | 39 | 1-1407 | 0.041 | 40 | 1-469 | −0.463 |

H5N1 (A/Jiangsu/1/2007)(Human)

| | | | | | | |
|---|---|---|---|---|---|---|
| PB1 | 41 | 1-2271 | 0.014 | 42 | 1-757 | −0.428 |
| HA | 43 | 1-1701 | 0.017 | 44 | 1-567 | −0.435 |
| NP | 45 | 1-1494 | 0.021 | 46 | 1-498 | −0.434 |
| NA | 47 | 1-1347 | 0.009 | 48 | 1-449 | −0.407 |

H7N2 (A/chicken/NJ/294508-12/2004)(Avian)

| | | | | | | |
|---|---|---|---|---|---|---|
| PB1 | 49 | 1-2271 | 0.006 | 50 | 1-757 | −0.444 |
| HA | 51 | 1-1656 | 0.036 | 52 | 1-552 | −0.377 |
| NP | 53 | 1-1494 | 0.024 | 54 | 1-498 | −0.457 |
| NA | 55 | 1-1359 | 0.013 | 56 | 1-453 | −0.491 |

H7N3 (A/Canada/rv504/2004)(Human)

| | | | | | | |
|---|---|---|---|---|---|---|
| PB1 | 57 | 1-2271 | 0.027 | 58 | 1-757 | −0.429 |
| HA | 59 | 1-1701 | 0.029 | 60 | 1-567 | −0.405 |
| NP | 61 | 1-1494 | 0.020 | 62 | 1-498 | −0.450 |
| NA | 63 | 1-1407 | 0.042 | 64 | 1-469 | −0.413 |

H7N7 (A/Netherlands/219/03)(Human)

| | | | | | | |
|---|---|---|---|---|---|---|
| PB1 | 65 | 1-2271 | 0.019 | 66 | 1-757 | −0.441 |
| HA | 67 | 1-1707 | 0.008 | 68 | 1-569 | −0.447 |
| NP | 69 | 1-1494 | 0.040 | 70 | 1-498 | −0.445 |
| NA | 71 | 1-1413 | −0.009 | 72 | 1-471 | −0.423 |

H9N2 (A/Hong Kong/1073/99)(Human)

| | | | | | | |
|---|---|---|---|---|---|---|
| PB1 | 73 | 1-2274 | 0.025 | 74 | 1-758 | −0.434 |
| HA | 75 | 1-1680 | 0.021 | 76 | 1-560 | −0.440 |
| NP | 77 | 1-1494 | 0.026 | 78 | 1-498 | −0.464 |
| NA | 79 | 1-1401 | 0.020 | 80 | 1-467 | −0.453 |

Generation of Synthetic Influenza Viruses

To attenuate an influenza virus, large parts of the coding regions of the PB1, NP, and HA genome segments of influenza virus A/PR/8/34 (“PR8”) were redesigned. The reference sequences of the 8 gene segments for this strain are available under genbank accession numbers AF389115 (segment 1, Polymerase PB2), AF389116 (segment 2, Polymerase PB1), AF389117 (segment 3, Polymerase PA), AF389118 (segment 4, hemagglutinin HA), AF389119 (segment 5, nucleoprotein NP), AF389120 (segment 6, neuraminidase NA), AF389121 (segment 7, matrix proteins M1 and M2), and AF389122 (segment 8, nonstructural protein NS1). An 8-plasmid ambisense system for this strain cloned in the vector pDZ (Quinlivan, M et al., 2005, J. Virol. 79, 8431) was obtained from Peter Palese and Adolfo Garcia-Sastre (Mt. Sinai School of Medicine).

Coding regions of the segments PB1, HA, and NP were targeted to be recoded. Nucleoprotein NP is a major structural protein and the second most abundant protein of the influenza virion (1,000 copies per particle) that binds as monomer to full-length viral RNAs to form coiled ribonucleoprotein. HA is on of two viral structural proteins protruding from the viral surface which mediating receptor attachment and virus entry. PB1 is a crucial component of the viral RNA replication machinery.

Without altering either amino acid sequence or the existing codon bias, the existing codons were rearranged to de-optimize codon pairs. A minimum of 120 nucleotides at either segment terminus were left unaltered. This resulted in hundreds of silent mutations per genome segment without any amino acid changes. The terminal 120 nucleotides at either end of the segment were not altered so as not to interfere with replication and encapsidation.

A nucleotide sequence encoding NP (SEQ ID NO:95) was synthesized by de-optimizing codon pairs between codons 27-460 (nucleotides 126-1425 of the NP segment) while retaining wildtype codon usage. NP^{min} (SEQ ID NO:97) contains 314 silent mutations. A nucleotide sequence encoding PB1 (SEQ ID NO:81) was synthesized by de-optimizing codon pairs between codons 169-488 (nucleotides 531-1488 of the PB1 segment) while retaining the wild type codon usage (PB1^{Min}). Segment PB1^{Min} (SEQ ID NO:85) contains 236 silent mutations compared the wt PB1 segment. A synonymous encoding of HA (SEQ ID NO:93) was synthesized by de-optimizing codon pairs between codons 50-541 (nucleotides 180-1655 of the HA segment) while retaining the wildtype codon usage (HA^{Min}). HA^{Min} (SEQ ID NO:95) contains 353 silent mutations compared the to wt HA segment.

The characteristics of the new synthetic genome segments and their changes in Codon Pair Bias (CPB) are summarized in Table 5. A comparison of the extent of their deoptimization with respect to the human ORFeome is illustrated in FIG. 4.

TABLE 5
Characteristics of “De-Humanized” Influenza Genome Segments

| | | | CPB of | Number |
|---|---|---|---|---|
| Gene | Deoptimized | CPB of wt | Deoptimized | of silent |
| Segment | Coding Region^{a} | Segment^{b} | Segment^{c} | Mutations |
| NP^{Min} | 125-1426 | 0.012 | −0.421 | 314 |
| PB1^{Min} | 519-1494 | 0.007 | −0.386 | 236 |
| HA^{Min} | 157-1654 | 0.019 | −0.420 | 353 |

^{a}nucleotide position within the genome segment that underwent the codon-pair deoptimization algorithm
^{b}original codon pair bias (CPB) of the corresponding wt sequence
^{c}codon pair bias (CPB) of the synthetic, codon pair-deoptimized gene segment

The deoptimized segments were synthesized de novo, and cloned into a standard ambisense, 8-plasmid system (E. Hoffmann, G. Neumann, Y. Kawaoka, G. Hobom, R. G. Webster, May 23, 2000, Proc. Natl. Acad. Sci. USA 97, 6108; J. H. Schickli et al., Dec. 29, 2001, Philos. Trans. R. Soc. Lond. B Biol. Sci. 356, 1965). To generate influenza viruses carrying one or more deoptimized segments, the respective plasmids carrying the recoded, synthetic segments, together with the complement of the remaining PR8 wt plasmids, were transfected into susceptible cells. 293T and Madin Darby Canine Kidney cells (MDCK) cells were obtained from the American Type Culture Collection (ATCC). Cells were grown in Dulbecco's modified Eagle's medium (Invitrogen), supplemented with 10% fetal bovine serum (HyClone) and penicillin-streptomycin (Invitrogen).

A total of 2 μg plasmid DNA (250 ng of each of 8 plasmids) was transfected into co-cultures of 293T and MDCK cells in 35 mm dishes using Lipofectamine 2000 (Invitrogen) according to manufacturers recommendations. After 6 hours of incubation at 37° C., the serum free Opti-MEM containing the transfection mix was replaced with DMEM containing 0.2% Bovine Serum Albumin (BSA). After a further 24 hours of incubation, 1 μg/ml TPCK-Trypsin was added to the dishes. Two days thereafter virus containing cell supernatants were collected and amplified on MDCK cells. Each deoptimized segment PB1^{Min}, NP^{Min}, and HA^{Min} in the background of the complementing 7 wt segments yielded a viable virus, as did any combination thereof, including that of all three deoptimized segments, giving rise to PR8-PB1/NP/HA^{Min} (abbreviated “PR8^{3F}”).

In Vitro Growth Characteristics and Titration of Synthetic Influenza Viruses

Several of these new synthetic viruses were analyzed for their in vitro growth characteristics in MDCK cells. The growth characteristics of codon-pair deoptimized synthetic viruses were analyzed by infecting confluent monolayers of MDCK cells in 100 mm dishes with 0.001 multiplicities of infection (MOI). Infected cells were incubated at 37° C. in DMEM, containing 0.2% Bovine Serum Albumin (BSA) and 2 μg/ml TPCK-Trypsin (Pierce, Rockford, Ill.). At the given time points 200 μl of supernatant was removed and stored at −80° C. until titration. Viral titers and plaque phenotypes were determined by plaque assay on confluent monolayers of MDCK cells in 35 mm six well plates using a semisolid overlay of 0.6% tragacanth gum (Sigma-Aldrich) in minimal Eagle medium (MEM) containing 0.2% Bovine Serum Albumin (BSA) and 4 ug/ml TPCK-Trypsin. After 72 hours of incubation at 37° C., plaques were visualized by staining the wells with crystal violet.

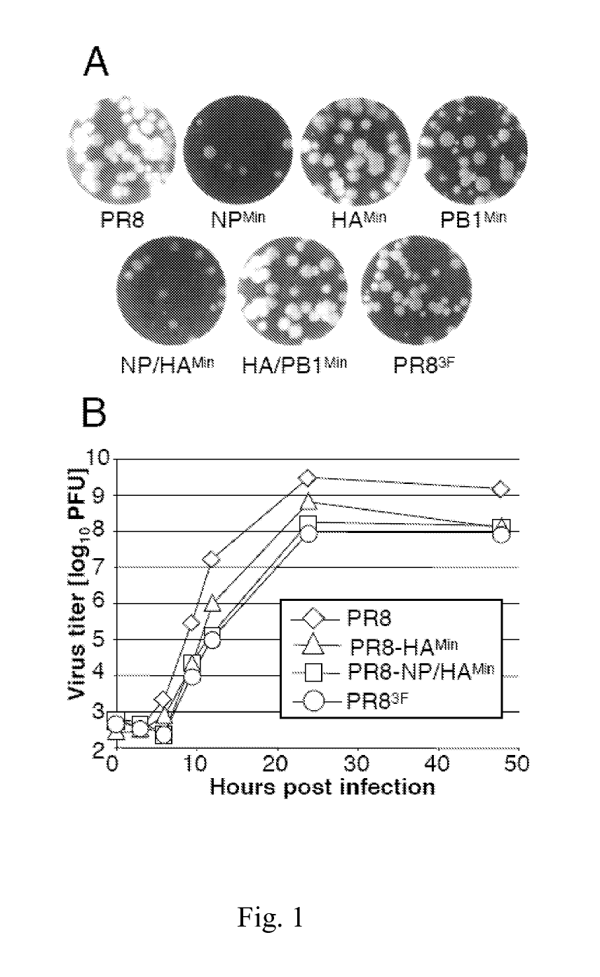

All mutant viruses formed plaques that were either indistinguishable from, or only slightly smaller than that of the wt virus (FIG. 1A). The mutant viruses grew less well than wt, but typically to only about ten-fold lower titers (FIG. 1B). The properties of viruses carrying combinations of synthetic segments other than depicted in FIG. 1 fall in between the curves or plaque phenotypes of PR8 and PR8^{3F} (data not shown).

In previous experiments we found codon-pair deoptimized polioviruses to have a greatly reduced specific infectivity (a lower PFU/particle ratio). Interestingly, this was not the case for deoptimized influenza viruses as their ratio of PFU to HA units was nearly identical to wt (data not shown).

Mouse Pathogenicity, In Vivo Virus Replication, and Vaccination

A minimum of 5 BALB/c mice (5-6 weeks old) per group were infected once by intranasal inoculation with doses ranging from 10⁰ to 10⁶ PFU of PR8^{3F} or of wt PR8. Inoculum virus was diluted in 25 μl PBS and administered evenly into both nostrils. A control group of 5 mice was inoculated with PBS only (mock). Venous blood from the tail vein was collected from all animals prior to initial infection for subsequent determination of pre-vaccination antibody titers.

Morbidity and mortality (weight loss, reduced activity, death) was monitored. The Lethal Dose 50 (LD₅₀) of the wildtype virus and the vaccine candidates was calculated by the method of Reed and Muench (Reed, L. J.; Muench, H., 1938, The American Journal of Hygiene 27: 493-497). Mice experiencing severe disease symptoms (rapid, excessive weight loss over 25%) were euthanized and scored as a lethal outcome.

For vaccination experiments mice were infected as above. 28 days following the initial infection (vaccination), venous blood from the tail vein was drawn for subsequent determination of post-vaccination antibody titers. The mice were then challenged with 10⁵ PFU of the wt virus PR8 corresponding to more than 1000 times the LD₅₀. Mortality and morbidity (weight loss, reduced activity, death) were monitored. The Protective Dose 50 (PD₅₀) of codon-pair deoptimized PR8^{3F} versus that of the PR8 was determined as the dose required to protect 50% of mice from a challenge with 1000×LD₅₀ of the wildtype virus, 28 days after a single inoculation with the vaccine virus.

To assess virus replication in the lungs of infected animals, BALB/c mice were infected intranasally with 10³ PFU of either PR8 or PR8^{3F}. At 1, 3, 5, 7, and 9 days post infection, the lungs of three mice were collected (wt infected mice did not survive beyond day 6). Lungs were homogenized in 1 ml of PBS and the virus titer per organ was determined by plaque assay on MDCK cells, as described above.

Despite their reasonably robust growth kinetics, codon pair deoptimized influenza viruses proved to be remarkably attenuated in mice (Table 6). Each individual deoptimized segment had a demonstrable effect on attenuation of the resulting virus, leading to a reduction in LD₅₀ of about 10, 30, and 500 fold, for PR8-NP^{Min}, PR8-HA^{Min}, and PR8-PB1^{Min}, respectively. Combining all three attenuating segments into one virus (PR8^{3F}) led to a cumulative attenuation of 13,000 fold (Table 6).

TABLE 6
Lethal Dose (LD₅₀) and Protective Dose (PD₅₀) of
Deoptimized Influenza Viruses

| Virus | LD₅₀ (PFU)^{a} | PD₅₀ (PFU)^{b} |
|---|---|---|
| PR8 (wt) | 6.1 × 10¹ | ~1.0 × 10^{0c} |
| PR8-NP^{Min} | 5.0 × 10² | n.d.^{d} |
| PR8-PB1^{Min} | 3.2 × 10⁴ | n.d. |
| PR8-HA^{Min} | 1.7 × 10³ | n.d. |
| PR8-NP/HA/PB1^{Min} | 7.9 × 10⁵ | 1.3 × 10¹ |
| (PR8^{3F}) | | |

^{a}The dose required to result in lethal disease in 50% of inoculated mice, calculated by the method of Reed and Muench (25).
^{b}The dose of vaccine required to protect 50% of mice with a single vaccination from a challenge infection with 1000 LD50 of the PR8 wt virus on day 28 post vaccination.
^{c}At the lowest of inoculum (1.0 × 100 PFU) 60% of mice were protected
^{d}not determined

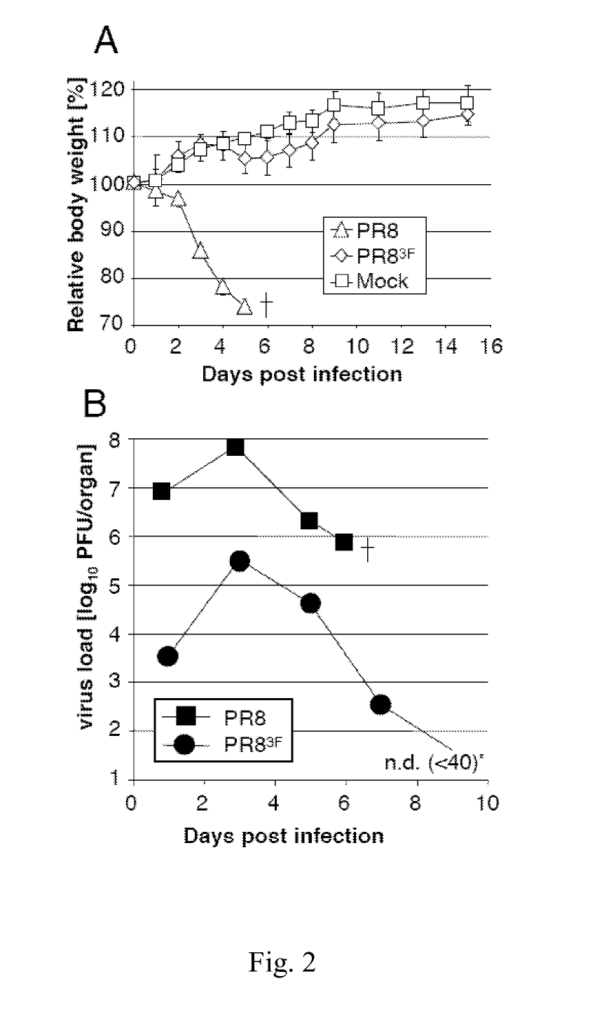

To test the pathogenic potential of codon pair deoptimized viruses in animals, BALB/c mice were infected intranasally with 10⁴ PFU of PR8^{3F} or PR8, and monitored for disease symptoms (ruffled fur, lethargy, and weight loss). At this dose, mice infected with wild-type PR8 developed severe symptoms with rapid weight loss and did not survive beyond day 5 of infection. Mice infected with PR8^{3F}, on the other hand, experienced no observable symptoms or weight loss, save for a small, transient delay in weight gain as compared to mock infected animals (FIG. 2A).

Live attenuated virus vaccines depend on a limited, yet safe, degree of replication within the host in order to effectively stimulate the immune system. To assess the replicative potential of a codon-pair deoptimized influenza virus in an immune competent animal host, we infected BALB/c mice intranasally with either 10³ PFU of PR8^{3F} or PR8 wild type virus, respectively. Within 24 hours, wt-infected mice were marked by 3000 fold higher viral load in their lungs compared to PR8^{3F}, setting the stage for lethal disease progression in under 6 days (FIG. 2B). Conversely, in PR8^{3F} infected animals, amplification of the vaccine virus progressed more slowly and peaked at a lower viral load than the wild type virus, resulting in a controlled course of infection with no overt disease symptoms, which eventually lead to virus clearance below detectable levels after nine days (FIG. 2B).

Infection by a sub-lethal dose of wild type virus can in principle accomplish the same immune protection as vaccination with an attenuated virus. In nature, wild type infections often result in protective immune responses, either after recovery from the disease, or even after a sub-clinical infection, a scenario representing the “natural” way of immunization. Indeed, the Chinese scholar Li Shizhen described the art of inoculating humans with live smallpox in his voluminous Compendium of Materia Medica (1593). This method of smallpox vaccination was practiced in China for centuries. This practice was known to be very dangerous because the ratio between lethal dose (LD) and protective dose (PD) of smallpox must be small.

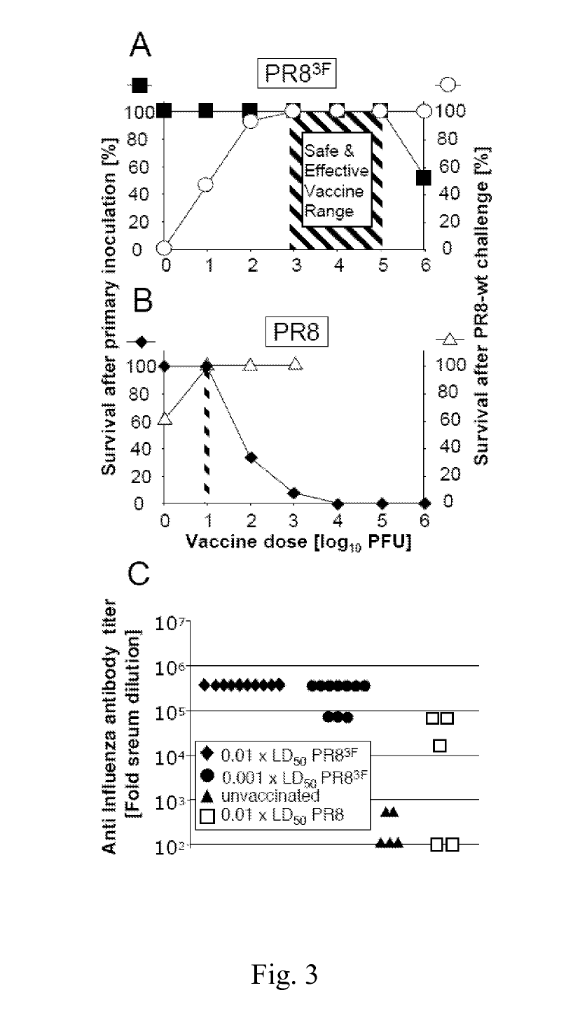

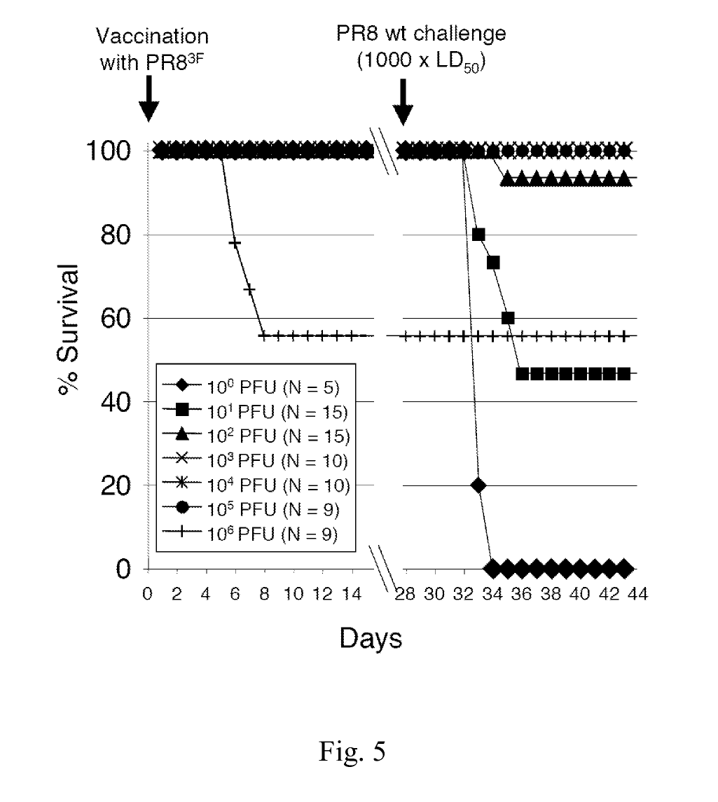

To address the issue of safety margin quantitatively with our influenza viruses, we determined the protective dose 50 (PD₅₀, the dose that provides protective immunity to half of the animals) of both PR8 and our most attenuated vaccine strain, PR8^{3F}. PR8 had a very low PD₅₀ of 1 PFU (due to its very robust replication kinetics in the infected animal). (Note that in the experiments described here, 1 PFU of PR8 virus, titered on MDCK cells, corresponds to approximately 40 virus particles (E. C. Hutchinson, M. D. Curran, E. K. Read, J. R. Gog, P. Digard, December 2008, J. Virol. 82, 11869). The LD₅₀ of PR8 was 61 PFU, resulting in an LD50/PD50 ratio of about 60. This ratio between the LD₅₀ dose and the PD₅₀ dose is the “safety margin” of a given virus if it were to be used as a vaccine. As expected, the safety margin of the wt (LD50/PD50=60) is very narrow—hence the wt is considered inadequate as a vaccine. In contrast, the attenuated virus PR8^{3F} had a PD₅₀ of 13 PFU, higher than the PD₅₀ of the wildtype virus, but still very low. Strikingly, the attenuated PR8^{3F} had an LD50 of 790,000 PFU and, thus, an LD50/PD50 ratio (safety margin) of 60,000, which is 1000-fold better than the wild-type virus (FIG. 3A versus FIG. 3B, shaded areas under the curve). Thus, it is easy to determine a dose of the attenuated virus PR8^{3F} that is both safe to administer and effective in inducing protective immunity, as is apparent also from the data presented in FIG. 5.

In a similar experiments, a single mouse vaccination at doses as high as 10⁶ TCID₅₀ of the cold adapted A/AA/6/60-ca (currently used as the FLuMist donor strain) did not provide protection against homologous challenge with the parental wild type A/AA/6/60 (G. A. Tannock, J. A. Paul, R. D. Barry, February 1984, Infect. Immun. 43, 457). These findings attest to the immunizing potential of a low-grade influenza virus infection in general, and to the safety profile of codon-pair deoptimized influenza viruses in particular. Combined with the expected high genetic stability of the underlying attenuating genetic changes (“death by a thousand cuts”) which form the basis of codon-pair deoptimization, this strategy may form the foundation of a new generation of live attenuated influenza virus vaccines.

Determination of Influenza-Specific Antibodies After Vaccination

Nunc Maxisorp ELISA 96 well plates were coated over night with 100 ng purified Influenza PR8 virus in 100 μl PBS followed by blocking with 100 μl 1% BSA in PBS. Serial 5-fold dilutions in PBS/1% BSA of mouse sera obtained prior to and 28 days after a single intranasal vaccination were incubated for 2 hours at room temperature. Mice were previously vaccinated with approximately 0.01 or 0.001×LD₅₀ of PR8^{3F} (10³ PFU or 10⁴ PFU, respectively), 0.01×LD₅₀ of PR8 wt (10⁰ PFU) or mock vaccinated. After 4 washes with PBS the wells were incubated with 1:500 of anti mouse-alkaline phosphatase conjugated secondary antibody (Santa Cruz) for another 2 hours at room temperature. Following 4 washes with PBS and brief rinsing with distilled water 100 μl of a chromatogenic substrate solution containing 9 mg/ml p-nitrophenylphosphate in 200 mM diethanolamine, 1 mM MgCl₂, pH 9.8 was added. The color reaction was stopped by addition of an equal volume of 500 mM NaOH. Absorbance at 405 nm was read using a Molecular Devices ELISA reader. The endpoint antibody titer was defined as the highest dilution of serum that gave a signal greater than 5 standard deviations above background. Background level was determined from wells processed identically to experimental samples, in the absence of any mouse serum.

The mean anti-influenza serum antibody titer in mice immunized with 0.01×LD₅₀ of the respective viruses was 312,500 for PR8^{3F} and 27,540 for PR8 (FIG. 3C). At an even lower and, thus, even safer vaccine dose of 0.001×LD₅₀ the immune response toward PR8^{3F} was nearly unchanged with an antibody titer of 237,500 (FIG. 3C). Thus, at identical doses relative to their respective LD50, PR8^{3F} is a much more potent inducer of influenza-specific antibodies.

Together with the exceptionally high growth kinetics in tissue culture (10⁸ PFU/ml) and the low protective dose of deoptomized influenza viruses, the SAVE technology sets the stage for making very cost efficient live attenuated influenza vaccines. 10 milliliter of culture supernatant contains enough virus to vaccinate and protect approximately 1 million mice with a single shot of 100 PD₅₀ doses of PR8^{3F} (FIG. 3A, FIG. 5).

We claim:

1. An attenuated influenza virus genome which comprises a nucleic acid encoding nucleoprotein (NP), and a nucleic acid encoding a polymerase protein, wherein the codon pair bias of each of said nucleic acids is less than the codon pair bias of a parent nucleic acid from which it is derived.

2. The attenuated influenza virus genome of claim 1, the polymerase protein is PB1.

3. The attenuated influenza virus genome of claim 1, which further comprises a nucleic acid encoding a virion protein, wherein the codon pair bias of the virion protein nucleic acid is less that the codon pair bias of a parent nucleic acid from which it is derived.

4. The attenuated influenza virus genome of claim 3, wherein the virion protein is hemagglutinin (HA).

5. The attenuated influenza virus genome of claim 1, wherein the parent nucleic acid is from a natural isolate.

6. The attenuated influenza virus genome of any one of claims 1 to 5, wherein the codon pair bias is reduced by shuffling the codons of the parent nucleic acid.

7. The attenuated influenza virus genome of claim 3, wherein the codon pair bias of one or more of the nucleic acids encoding nucleoprotein (NP), the virion protein, and the polymerase protein is at least 0.05 less than the codon pair bias of the parent nucleic acid.

8. The attenuated influenza virus genome of claim 3, wherein the codon pair bias of one or more of the nucleic acids encoding nucleoprotein (NP), the virion protein, and the polymerase protein is less that −0.1.

9. The attenuated influenza virus genome of claim 3, wherein the codon pair bias of one or more of the nucleic acids encoding nucleoprotein (NP), the virion protein, and the polymerase protein is less that −0.2.

10. The attenuated influenza virus genome of claim 3, wherein the codon pair bias of one or more of the nucleic acids encoding nucleoprotein (NP), the virion protein, and the polymerase protein is less that −0.3.

11. The attenuated influenza virus genome of claim 3, wherein the codon pair bias of one or more of the nucleic acids encoding nucleoprotein (NP), the virion protein, and the polymerase protein is less that −0.4.

12. An attenuated influenza virus which comprises the attenuated influenza virus genome of any one of claims 1 to 5.

13. The attenuated influenza virus of claim 12, wherein the attenuated influenza virus infects a human.

14. The attenuated influenza virus of claim 12, wherein the attenuated influenza virus infects a bird.

15. The attenuated influenza virus of claim 12, wherein the attenuated influenza virus infects a pig.

16. A vaccine composition for inducing a protective immune response in a subject, wherein the vaccine composition comprises a nucleic acid encoding nucleoprotein (NP) and a nucleic acid encoding a polymerase protein, wherein the codon pair bias of each of said nucleic acids is less than the codon pair bias of a parent nucleic acid from which it is derived.

17. The vaccine composition of claim 16, which further comprises a nucleic acid encoding a virion protein, wherein the codon pair bias of the virion protein nucleic acid is less that the codon pair bias of a parent nucleic acid from which it is derived.

18. A method of eliciting a protective immune response in a subject comprising administering to the subject a prophylactically or therapeutically effective dose of the vaccine composition of claim 16.

19. The method of claim 18, further comprising administering to the subject at least one adjuvant.

20. A method of making an attenuated influenza virus genome comprising:
a) obtaining the nucleotide sequence encoding the nucleoprotein (NP) of an influenza virus and the nucleotide sequence encoding a polymerase protein of an influenza virus;
b) rearranging the codons of the nucleotide sequences to obtain mutated nucleotide sequences that
i) encode the same amino acid sequences as the unrearranged nucleotide sequences, and
ii) have a reduced codon pair bias compared to the unrearranged nucleotide sequence; and
c) substituting all or part of the mutated nucleotide sequences into the unrearranged nucleotides of the influenza virus genome.

21. The method of claim 20, which further comprises obtaining the nucleotide sequence encoding a virion protein of an influenza virus;
b) rearranging the codons of the nucleotide sequence to obtain a mutated nucleotide sequence that
i) encodes the same amino acid sequence as the unrearranged nucleotide sequences, and
ii) has a reduced codon pair bias compared to the unrearranged nucleotide sequence; and
c) substituting all or part of the mutated nucleotide sequences into the unrearranged nucleotides of the influenza virus genome.

22. The method of claim 21, wherein polymerase protein is PB1 and the virion protein is hemagglutinin (HA).


SEQUENCE LISTING (103 sequences)

SEQ ID NO: 1 - DNA, 2271 bp, Influenza A virus
atggatgtcaatccgactttacttttcttgaaagttccagcgcaaaatgccataagcaccacattcccgtatactggagatcctccatacagccatggaacaggaacaggatacaccatggacacggttaacagaacacatcaatattcagaaaaggggaaatggacaacaaactcagaaactggagcaccccaacttaacccaattgatggaccactacctgaggacaatgagccaagtggatatgcacaaacggactgtgtccttgaagcaatggctttccttgaagagtcccacccaggaatctttgaaaactcgtgtcttgaaacgatggaagttgttcaacaaacaagagtggacaaattgacccaaggccgtcagacctatgattggacattaaacaggaatcagccggctgcaactgcattagctaataccatagaggtcttcagatcgaacggtctgacagctaatgactcaggaaggctaatagattttctcaaggatgtgatggaatcaatggataaagaggaaatggaaataacaacgcatttccaaaggaaaagaagagtgagagacaacatgaccaagaaaatggtcacacaaagaacaataggaaagaagaagcagagactaaacaaaaggagctatctaataagagcattgacattgaacacaatgacaaaagacgccgaaagaggcaaattaaggagaagagcaattgcaacacccggaatgcaaatcagaggatttgtatactttgttgaaacattagcaaggagcatttgtgagaagcttgaacaatctggactcccagttggaggcaatgaaaagaaggctaaactggcaaatgttgtgagaaaaatgatgactaattcacaagacacagaactctctttcacaatcactggagacaacaccaaatggaatgaaaatcagaatcctaggatgtttctggcgatgataacatatataacaagaaaccaacctgaatggttcaggaatgtcttgagcattgcacctataatgttctcaaacaaaatggcaagactagggaaaggatacatgttcgaaagtaagagcatgaagcttcgaacacaaataccggcagaaatgctagcaagtattgatctgaaatatttcaatgagtcaacaagaaagaagatagagaagataaggcctcttctaatagatggtacagcctcattgagccccggaatgatgatgggcatgttcaacatgctaagtacagttttgggagtttcgattctaaatctagggcaaaagaggtacaccaaaacaacatactggtgggacggactccaatcctctgatgactttgctctcatagtgaatgctccgaatcatgagggaatacaagcaggagtagacagattctatagaacctgcaagctggtcggaatcaacatgagcaaaaagaagtcctacataaataggacaggaacatttgaattcacaagttttttctatcgctatggatttgtagccaacttcagcatggagttgcccagctttggagtgtctgggattaatgaatctgcagacatgagcattggagtgacagtgataaagaacaacatgataaacaatgaccttggaccagcaacagctcaaatggctcttcagctgttcatcaaggactacagatacacatatcggtgccacagaggagatacacaaattcagacaagaaggtcattcgagctgaagaagttgtgggaacaaacccgctcaaaagcaggactgctggtctcagatggaggaccaaatctatacaatatccggaatctccatattccggaagtctgcttgaaatgggagctaatggacgaagactatcagggaaggctttgtaaccccctgaatccatttgtcagccacaaagagatagagtctgtaaacaatgctgtggtgatgccagctcatggcccagccaagagcatggaatatgatgctgttgctaccacgcactcctggatccctaaaaggaaccgctccatcctcaatacaagccaaaggggaatccttgaagatgaacagatgtatcaaaagtgctgcaatctattcgagaaattcttccctagcagttcatacaggaggccggttgggatttccagcatggtggaggccatggtttctagggcccgaattgatgcgcgaattgacttcgaatctggacggattaagaaggaggagtttgctgagatcatgaagatctgttccaccattgaagagctcagacggcagaag

SEQ ID NO: 2 - DNA, 2271 bp, Unknown
  deoptimized Influenza A Virus
atggacgttaaccctacactattgttccttaaggtgccagcccaaaacgctatatccacaacattcccatataccggagacccaccatactcacacggaaccggaaccggatacacaatggataccgttaataggacacaccaatatagcgaaaagggaaaatggacaacgaatagcgaaacaggcgcaccgcaattgaatccgatagacggaccgttacccgaagataacgaacctagcggatacgcacaaaccgattgcgtactcgaagctatggcatttctcgaagagtcacatcccgggatattcgagaatagttgccttgagacaatggaggttgtgcaacagactagggtcgacaaactgacacaggggagacagacatacgattggacactgaataggaaccaacctgccgcaaccgcacttgcgaatacaatcgaagtgtttaggtctaacggactaaccgcaaacgatagcggaagactaatcgatttccttaaagacgttatggagtctatggacaaagaggagatggagattacgacacatttccaacgaaaaagacgcgttagggataatatgacaaaaaagatggttacacaacggacaatcggtaagaaaaagcaacggttgaacaaacggtcatacttgattagggcactaacattgaatacaatgactaaggacgccgaaaggggaaagcttagacgacgcgcaatcgctacaccaggaatgcaaattaggggattcgtgtatttcgtcgagacactcgctaggtcaatttgcgaaaaactcgagcaatccggattgccagtcggcggaaacgagaaaaaggctaagcttgcgaacgtagtgagaaagatgatgacaaattcccaagataccgaactatcttttacgataaccggagataatacgaaatggaacgaaaaccaaaaccctagaatgtttctcgcaatgattacatatataacacgtaaccaacccgaatggtttagaaacgttctgtcaatcgctcctattatgtttagcaataagatggctagactaggtaaggggtatatgttcgaatctaagagtatgaagcttaggacacagatacctgccgaaatgttagctagcatagaccttaagtactttaacgaatcgactagaaagaaaatcgaaaagattagaccactactgatagacggaaccgctagcctatcccccggaatgatgatgggaatgttcaatatgctatcgacagtgttaggcgtaagcatactgaatctcggacagaaaagatatacaaagacaacatattggtgggacggactgcaatctagcgacgatttcgcactaatcgttaacgcacctaatcacgaagggatacaagccggagtcgataggttttacagaacatgtaagttagtcggaataaatatgagtaagaaaaagtcatacataaatagaaccggaacattcgaatttacaagctttttttatagatacggattcgttgcgaatttctcaatggagttaccgtcattcggagtgagcggaattaacgaatccgccgatatgtcaatcggagtgacagtgataaagaataatatgattaacaacgatctcggaccagctaccgcacaaatggcactacaattgttcattaaagactataggtatacatatagatgccataggggcgatacacagatacagactagaaggtcattcgaactgaaaaagttgtgggagcaaactaggtctaaggccggattgttggtaagcgacggaggccctaatctgtataatattaggaatctgcatatacccgaagtgtgtcttaaatgggagcttatggacgaagactatcaggggagactatgtaacccacttaatccattcgttagccataaagagatagagtccgttaataacgcagtcgttatgccagcacacggaccggctaagtctatggaatacgacgcagtcgcaacgacacatagttggataccgaaacggaatagatcgatactgaatactagccaacgcggaatactcgaagacgaacaaatgtatcaaaagtgttgtaatctattcgaaaagttctttccgtcaagctcatacagacgaccagtcggaattagctctatggtcgaggctatggtgagtagagctagaatcgacgctagaatcgatttcgaatccggaaggattaaaaaagaggaattcgcagagattatgaagatttgcagtacaatcgaagagcttaggagacagaaa

SEQ ID NO: 3 - DNA, 1683 bp, Influenza A Virus
atgtacaaaatagtactagtacttgcgctccttggagcggtgcatggtcttgacaaaatatgccttggacatcatgcagtccccaatggcaccatcgtaaagactctcacaaacgaaaaggaagaggtgaccaatgctactgaaacggtggaaagtaaaagcctggacaaactttgcatgaaaagtcggaattacaaggacctaggtaattgccacccgatagggatggtgatagggactcctgcttgtgacttacacctcaccggaacatgggacactttgatagagagagacaattccattgcctactgttacccaggtgccactgtgaatgaagaagcattaaggcagaaaattatggaaagtggagagattgacaagataagcaccgggtttacatatgaatcatccatcaatccagctggaaccactaaagcatgcatgagaaatgggaaaaacagtttctatgcagagctaaagtggctagtgtcgaaggacaaaggacggaacttcccacaaacaacaaacacatacaggaatacagattcaacagaacaccttataatctggggaattcatcacccgtcaagcacacaagaaaagaatgatctgtatggaacacaatcactttccatttcagtagggagttctacttatcaaaacaactttgtgcctgtggtgggagcaagaccacaggtgaatggccaaagtgggcggattgatttccattgggcgatggtacaaccgggtgataacatcactttttcgcataacggcggactaatagcacctagtagagtgagtaaactaaagggaagaggccttggcattcaatcaggagcttcagtagataatgactgtgaatcaaaatgtttttggaaaggtggatccatcaacaccaaactcccttttcagaatctttccccaagaactgtgggtcaatgccccaagtatgtgaacaaaaagagcctgttgcttgctaccggaatgaggaatgtgccagaggttgtccaaggaagaggcctgttcggagcaattgctggattcatagaaaatggatgggaagggatggtagatggttggtatggtttccgacatcaaaatgcccaaggcactggtcaggctgcggattacaaaagcactcaggcagctatagatcaaatcaccgggaaattgaacagactgatagagaagacaaacacagagttcgaatccatagaatctgagttcagtgaaattgaacatcaaattggcaatgtaataaactggactaaggattcgataacagacatttggacgtatcaagctgaattactggtagcaatggaaaaccagcatacaatcgacatggctgattcagaaatgctgaatctatatgagagagtgaggaagcaactgaggcaaaatgcagaagaagatgggaaagggtgctttgaaatatatcacaaatgcgacgacaactgcatggaaagcatcagaaacaacacctatgaccatacacaatacagagaagaagcactcttgaacagactcaacattaatccggtgaaactctcttctgggtacaaagatgttatactgtggtttagcttcggggcgtcatgctttgtacttttggctgtcatcatggggcttgttttcttctgtctgaaaaatggaaacatgcgatgcacaatctgtatt

SEQ ID NO: 4 - DNA, 1683 bp, Unknown
  deoptimized Influenza A Virus
atgtataagatagtgctcgtactcgcactattaggcgcagtgcacggactcgacaaaatttgcctagggcatcacgcagtgcctaacggaactatcgttaagacacttactaacgaaaaagaggaagtgactaacgctaccgaaacagtcgaatcaaaatcactcgacaaattgtgtatgaaaagtcggaattataaagacctaggcaattgccatccgatagggatggtgatagggactcccgcttgcgatctgcatctgacagggacatgggatacacttatcgaacgggacaatagtatagcgtattgttatccaggcgctacagtgaacgaagaggcacttagacaaaaaattatggaatccggcgaaatcgataagattagtaccggattcacatacgaatcctctattaatcccgcaggaacaactaaggcttgtatgcgaaacggtaagaattcgttttacgctgaactgaaatggcttgtgagtaaggacaaaggtaggaatttcccacaaactactaatacttataggaataccgattcaaccgaacatctgattatatgggggatacaccatccaagttcgacacaagagaaaaacgatctatacggaacgcaatcccttagcattagcgtagggtctagtacttatcagaataatttcgtaccggtagtgggcgctagaccgcaagtgaacggacaatccggtagaatcgatttccattgggctatggtgcaaccaggcgataacataacttttagccataacggcggactgatagcgcctagtagagtgagtaagcttaagggaagggggttggggatacaatccggcgctagcgtagacaacgattgcgaatcaaaatgcttttggaaaggggggtcaattaatactaaattgccatttcagaatctgtcacctagaacagtgggacaatgccctaaatacgttaataagaaaagtctgttactcgcaaccggtatgcgaaacgtaccagaggtagtgcaaggtagggggctattcggagcgatagcgggatttatcgaaaacggatgggagggtatggtcgacggatggtacgggtttagacaccaaaacgcacagggaaccggacaggcagcagactataaatcgacacaagccgctatagaccaaattaccggtaagcttaacagactgatcgaaaagactaataccgaattcgaatcaatcgaatccgaatttagcgaaatcgaacaccaaatcggaaacgtaattaattggacaaaagactcaattaccgatatatggacatatcaagccgaactgttagtcgctatggagaatcagcatacaatcgatatggccgatagcgaaatgcttaacctttacgaaagggtgagaaaacagcttagacaaaacgctgaagaggacggtaaggggtgtttcgaaatataccataaatgcgacgataattgtatggagtctatacggaataacacatacgaccatacgcaatatagagaggaagcactactgaatagacttaacattaatccggttaagctatctagcggatataaagacgtgatattgtggttctcattcggagcgtcatgtttcgtattgctcgcagtgattatgggactcgtattcttttgccttaaaaacggtaatatgagatgcacaatttgcata

SEQ ID NO: 5 - DNA, 1494 bp, Influenza A Virus
atggcgtctcaaggcaccaaacgatcttatgaacaaatggaaactggtggggaacgccagaatgccactgaaatcagagcatctgttgggagaatggttggcggaatcgggagattctacatacagatgtgcactgagctcaaactcagtgactacgaagggagactgatccaaaacagcataaccatagagaggatggttctctcggcatttgatgagaggagaaacaagtatctggaggagcatcccagtgctgggaaagatcccaagaagactggaggtccaatctacaggaggagagatggcaaatggatgagagagttgatcctatatgacaaagaagagatcagaagaatttggcgtcaagctaataatggagaggacgcaactgctggtctcacccatttgatgatttggcattccaatctgaatgatgccacataccagagaacaagggcacttgtgcgtactggaatggaccctaggatgtgctctctgatgcaaggctcaacccttccgaggagatctggggctgctggagcggcagtgaaaggggttggaacaatggtgatggaattgatccggatgatcaagcgagggatcaatgatcggaatttctggagaggcgaaaatggacggagaactagaattgcctacgagagaatgtgcaacatcctcaagggaaaattccaaacagcagcacaacgagcaatgatggaccaagtgagggaaagccggaatcctgggaatgctgaaattgaagatctcatctttctcgcacggtctgctctcatcctgaggggatcagtggctcataagtcctgcctgcctgcttgtgtgtacggacttgctgtagccagtggatatgactttgaaagagaggggtactctctagtcggaattgatcctttccgtctgctccaaaacagtcaagtcttcagtctcatcagatcaaacgaaaatccagcgcataaaagtcagctggtatggatggcatgccactctgcagcattcgaagatctgagagtgtcaagcttcatcagaggaacaagagtagtcccaagaggacaactgtccaccagaggagttcagattgcttcaaatgagaacatggagacaatggactccagtactcttgaattgaggagcagatactgggctataagaacaagaagcggagggaacaccaaccaacagagagcatctgcaggacaaatcagcgtacagcccacattctctgtgcagagaaacctcccattcgagagagcaaccattatggcagcatttacaggaaacactgaaggcagaacttcagacatgagaactgagatcataaggatgatggaaaatgccagacctgaagatgtgtctttccaggggcggggagtcttcgagctctcggacgaaaaggcaacgaacccgatcgtgccttcctttgacatgagtaatgaagggtcttatttcttcggagacaatgcagaggagtatgacaat

SEQ ID NO: 6 - DNA, 1494 bp, Unknown
  deoptimized Influenza A Virus
atggctagtcagggaactaagcgatcttacgaacagatggagacagggggggaaagacagaacgctaccgaaattagggctagcgtagggagaatggtaggcggaatcggaagattctatatccaaatgtgcactgagcttaagctatccgattacgaaggaagactgatacagaattcgatcacaatcgaacgtatggtgcttagcgcattcgacgaaagacgtaataagtatctcgaagagcatcctagcgcaggtaaggaccctaaaaaaacaggcggacctatctatagacgtagagacggtaagtggatgagagagttgatactgtacgataaagaggagatacggagaatctggagacaagcgaataacggcgaagacgctaccgccggactgacacatctgatgatttggcactctaatctgaacgacgcaacatatcaacggactagggcactcgttagaaccggaatggaccctagaatgtgttcactcatgcagggatctacactccctagaaggtccggagccgcaggcgcagccgttaagggagtcggaactatggttatggagttgatcagaatgatcaaaagagggattaacgataggaatttctggagaggcgaaaacggaagacggactagaatcgcatacgaacgaatgtgcaatatccttaagggaaaatttcagaccgctgcgcaacgcgctatgatggaccaagtgagagagtcacggaatcccggtaacgccgaaatcgaagacctaatctttctcgctagatccgcactgatactcagggggtcagtcgcacataaatcatgcttgcccgcatgcgtttacggactcgcagtcgcatccggatacgatttcgagagagaggggtatagtctcgtcggaatcgatccattcagattgctccagaatagtcaggtgttctcactgattaggtctaacgagaatcccgcacataaatcgcaactcgtatggatggcatgccatagcgctgcattcgaagaccttagagtgagtagtttcattagggggactagggtggtgcctagagggcaactgtctactaggggggtgcaaatcgctagtaacgagaatatggagacaatggactctagtactctcgaactcagatctaggtattgggcaatcagaacaagatccggagggaatacgaatcagcaacgggctagcgcagggcaaattagcgtgcaaccaacatttagtgtgcaacggaatctgccattcgaaagggctactattatggccgcatttaccggaaataccgaagggagaacctctgatatgcgaactgagataatcagaatgatggagaacgctagaccagaagacgtgtctttccaagggagaggcgtattcgaactgtctgacgaaaaagcgactaatccgatcgttccgtcattcgatatgtctaacgagggatcttactttttcggagataacgcagaggaatacgataat

SEQ ID NO: 7 - DNA, 1410 bp, Influenza A virus
atgaatcctaatcaaaaattattcgcactctctggggtggccatagcactgagtatcctcaacctactaataggaatatccaatgtgggactgaatgtctcactacacctgaagggaagcagtgaccaggataagaattggacatgcacgagtgtaacacaaaccaacacgactttaatcgaaaacacgtatgtcaacaataccactgtcatcaataaggaaacagggactacaaagcaaaattatctaatgctgaacaagagtttatgcaaagttgaaggatgggtagtggtggccaaggacaatgccataagattcggtgaaagtgaacaaataatagtgacaagggagccgtatgtgtcatgtgatccattaggatgtaagacgtacgcactgcatcaagggacaaccattagaaacaagcactcaaacggaacaatacacgacaggactgctttcagagggttgatatcaactcctttggggagcccccctgtagtcagcaatagtgactttctttgtgtagggtggtcaagcaccagttgccatgacggcatcgggcggatgaccatttgcgtgcagggaaataataacaacgcaacagctacagtgtactatgaccgaaggctcactaccacaataaaaacatgggcagggaaaatccttaggacgcaagagtcggaatgtgtatgccacaatggaacatgtgtagtaataatgaccgatggatcggcaagcagccaggcacatacaaaagttctgtatttccacaaaggactagtaataaaagaggaagccctcaagggatcagccagacacatagaggagtgctcatgctatgggcacaattcaaaggtgacttgtgtatgcagggacaactggcaaggagccaatagaccagtgattgaaatagatatgaatgccatggagcatacaagtcagtatctatgtacaggagttctcactgacacgagcagaccatcagacaaatcaatgggcgactgtaataatccgatcactgggagtccgggagcccctggggtcaaaggattcggcttcctggatagtgacaatacatggttgggccgcacaataagtcctcgttccaggagtggttttgagatgttgaagatacctaatgctgggacagacccaaattctagaatcactgagaggcaagaaatagttgacaacaacaattggtcaggatactcaggaagtttcattgactattgggatgaaagcagtgtgtgctacaacccctgtttttatgttgaattaataagaggaaggcctgaagaagccaagtatgtttggtggacgagcaacagtttagttgcactatgtggaagcccaatctcagttgggtccggttccttccccgatggggcacaaatccaatacttttcg

SEQ ID NO: 8 - DNA, 1410 bp, Unknown
  Deoptimized Influenza A virus
atgaatcctaaccaaaagctattcgcactaagcggagtcgccatagccctatcaatactgaatctgttaatcggaatatcgaacgttgggttgaacgttagtttgcaccttaaggggtcatccgaccaagacaaaaattggacatgtactagcgttacgcaaacaaatacgactttgatcgaaaatacatacgttaacaatacgacagtgataaataaagagaccggaactactaagcaaaactatctgatgctgaataagtcactatgtaaggtcgagggatgggtggtagtcgctaaagacaacgcaataaggttcggcgaaagcgaacagataatcgtgacacgcgaaccatacgttagttgcgatccgttagggtgtaagacatacgcattacaccaagggactacgatacggaataaacactctaacggaacgatacacgacagaaccgcatttagggggttgatatcgacacctctcggatcacctcccgtagtgagtaatagcgatttcttatgcgtggggtggtcaagtactagttgtcacgacggaatcggacgtatgacaatatgcgtacaggggaataacaataacgcaaccgcaacagtgtattacgataggagactgactacaacaattaagacttgggccggtaagatactgagaacacaggaaagcgaatgcgtttgccataacggtacatgcgtagtgattatgacagacggatccgcaagttcgcaagcccatacgaaagtgctatattttcacaaagggctcgtaatcaaagaggaagcccttaagggatccgctagacatatcgaagagtgtagttgttacggacacaatagtaaggttacatgcgtatgtagggacaattggcaaggcgcaaatagaccagtgatagagatagacatgaacgctatggagcatacgagtcagtatctatgtaccggagtgttaaccgacactagtagacctagcgataagagtatgggcgattgcaataatccgataaccggatcacccggagcaccaggcgttaaggggttcgggtttctcgatagcgataatacatggttaggtaggacaatctcacctaggtcaagatccggattcgaaatgctcaaaatccctaacgccggaacagaccctaatagtaggattaccgaacgacaagagatagtcgacaataacaattggtcagggtatagcggatctttcatagactattgggacgaatcaagcgtatgttataacccatgtttctatgtcgaactgattagggggagacccgaagaggccaaatatgtgtggtggactagtaatagtctcgtagccctatgcggatcaccgataagcgtagggtcagggtcattcccagacggagcccaaatccaatattttagt

SEQ ID NO: 9 - DNA, 2271 bp, Influenza A virus
atggatgtcaatccgactctacttttcctaaaaattccagcgcaaaatgccataagcaccacattcccttatactggagatcctccatacagccatggaacaggaacaggatacaccatggacacagtaaacagaacacaccaatactcagaaaagggaaagtggacgacaaacacagagactggtgcaccccagctcaacccgattgatggaccactacctgaggataatgaaccaagtgggtatgcacaaacagactgtgttctagaggctatggctttccttgaagaatcccacccaggaatatttgagaattcatgccttgaaacaatggaagttgttcaacaaacaagggtagataaactaactcaaggtcgccagacttatgattggacattaaacagaaatcaaccggcagcaactgcattggccaacaccatagaagtctttagatcgaatggcctaacagctaatgagtcaggaaggctaatagatttcttaaaggatgtaatggaatcaatgaacaaagaggaaatagagataacaacccactttcaaagaaaaaggagagtaagagacaacatgaccaagaagatggtcacgcaaagaacaatagggaagaaaaaacaaagactgaataagagaggctatctaataagagcactgacattaaatacgatgaccaaagatgcagagagaggcaagttaaaaagaagggctatcgcaacacctgggatgcagattagaggtttcgtatactttgttgaaactttagctaggagcatttgcgaaaagcttgaacagtctgggctcccagtagggggcaatgaaaagaaggccaaactggcaaatgttgtgagaaagatgatgactaattcacaagacacagagatttctttcacaatcactggggacaacactaagtggaatgaaaatcaaaatcctcgaatgttcctggcgatgattacatatatcaccagaaatcaacccgagtggttcagaaacatcctgagcatggcacccataatgttctcaaacaaaatggcaagactagggaaagggtacatgttcgagagtaaaagaatgaagattcgaacacaaataccagcagaaatgctagcaagcattgacctgaagtacttcaatgaatcaacaaagaagaaaattgagaaaataaggcctcttctaatagatggcacagcatcactgagtcctgggatgatgatgggcatgttcaacatgctaagtacggtcttgggagtctcgatactgaatcttggacaaaagaaatacaccaagacaatatactggtgggatgggctccaatcatccgacgattttgctctcatagtgaatgcaccaaaccatgagggaatacaagcaggagtggacagattctacaggacctgcaagttagtgggaatcaacatgagcaaaaagaagtcctatataaataagacagggacatttgaattcacaagctttttttatcgctatggatttgtggctaattttagcatggagctacccagctttggagtgtctggagtaaatgaatcagctgacatgagtattggagtaacagtgataaagaacaacatgataaacaatgaccttggacctgcaacggcccagatggctcttcaattgttcatcaaagactacagatacacatataggtgccataggggagacacacaaattcagacgagaagatcatttgagttaaagaagctgtgggatcaaacccaatcaaaggtagggctattagtatcagatggaggaccaaacttatacaatatacggaatcttcacattcctgaagtctgcttaaaatgggagctaatggatgatgattatcggggaagactttgtaatcccctgaatccctttgtcagtcataaagagattgattctgtaaacaatgctgtggtaatgccagcccatggtccagccaaaagcatggaatatgatgccgttgcaactacacattcctggattcccaagaggaatcgttctattctcaacacaagccaaaggggaattcttgaggatgaacagatgtaccagaagtgctgcaatctattcgagaaatttttccctagcagttcatataggagaccggttggaatttctagcatggtggaggccatggtgtctagggcccggattgatgccagggtcgacttcgagtctggacggatcaagaaagaagagttctctgagatcatgaagatctgttccaccattgaagaactcagacggcaaaaa

SEQ ID NO: 10 - DNA, 2271 bp, Unknown
  Deoptimized Influenza A virus
atggacgtaaatcctacactgttattccttaagatacccgcacaaaacgctattagtacaacattcccatacacaggcgatccaccatactcacacggaaccggaaccggatacactatggatacagtgaataggacacaccaatatagcgaaaagggtaagtggactactaataccgaaacaggcgcaccacaactgaatccgatagacggacctctaccagaggataacgaacctagcggatacgcacaaaccgattgcgtacttgaggcaatggcctttctcgaagagtcacatccagggatattcgaaaactcatgcttagagactatggaggtcgtgcaacagactagagtcgataagcttacgcaagggagacagacatacgattggacactgaataggaatcaacctgccgcaaccgcactagccaatacaatcgaagtgtttcggtctaacggactaaccgctaacgaatccggtaggttgatcgatttccttaaggacgtaatggagtcaatgaataaggaggagatagagataacgacacatttccaacgaaagagacgcgttagggataatatgacaaaaaaaatggttacgcaacggacaatcggaaaaaaaaaacagagactaaataagagagggtatctaattagggcactaacacttaacacaatgactaaggacgccgaaaggggtaagcttaagagacgcgcaatcgctacacccggaatgcaaattagggggttcgtatatttcgttgagacactcgctagatccatttgcgaaaagcttgagcaatccggattgcccgtaggcggaaacgaaaaaaaggctaagctagcgaacgtagtgagaaagatgatgacaaattcacaggataccgagataagcttcacaattacaggcgataatactaagtggaacgagaatcagaatcctagaatgtttctcgcaatgattacatacataactagaaaccaacccgaatggtttagaaacatactgtcaatggcccctattatgttttcgaataagatggctagactcggaaaggggtatatgttcgaatcgaaacgtatgaaaattaggacacagatacctgccgaaatgttagcctcaatcgatcttaagtattttaacgaatcaacgaaaaaaaaaatcgaaaagataagaccgttactgatagacggaaccgcaagcctatcaccagggatgatgatgggaatgtttaatatgttaagtacagtgttaggcgtaagtatactgaatctagggcaaaaaaaatacactaagacaatatattggtgggacggactgcaatcaagcgacgatttcgcactaatcgttaacgcacctaatcacgagggaattcaggccggagtcgatagattttatagaacatgtaagttagtcggaattaatatgtctaagaaaaagtcatacattaacaaaaccggaacattcgaatttacaagctttttctatagatacggattcgtagcgaattttagtatggagttaccgtcattcggagtgagcggagtgaacgaatccgccgatatgtcaataggcgtaaccgtaattaagaacaatatgattaataacgatctcggacccgcaaccgctcaaatggcactacaattgttcattaaggattatcggtatacatatagatgccataggggggatacacagatacaaactagacggtctttcgagcttaaaaagctatgggatcaaacgcaatctaaagtcggactgttagttagcgacggagggcctaacctatacaatattaggaatctgcatatacccgaagtgtgtctaaaatgggagttaatggacgacgattatagggggagactatgcaatccacttaatccattcgttagtcataaagagatagatagcgttaataacgccgtagtgatgcctgcacacggaccagctaagtctatggagtacgacgcagtcgcaacaacgcatagttggataccgaaacggaatagatctatactgaatactagccaaagggggatactcgaagacgaacaaatgtaccaaaagtgttgcaatctattcgaaaaattttttccatctagctcatacagaagacccgtagggattagctctatggtcgaggcaatggtgagtagggctagaatcgacgctagagtcgatttcgaatccggtaggattaaaaaggaagagtttagcgagattatgaagatttgctctacaatcgaagagcttagacgacaaaaa

SEQ ID NO: 11 - DNA, 1698 bp, Influenza A virus
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggacatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctgggtctggtattatcatttcagatacaccagtccacgattgcaatacaacttgtcagacacccaagggtgctataaacaccagcctcccatttcagaatatacatccgatcacaattggaaaatgtccaaaatatgtaaaaagcacaaaattgagactggccacaggattgaggaatgtcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatt

SEQ ID NO: 12 - DNA, 1698 bp, Unknown
  Deoptimized Influenza A virus
atgaaagcgattctagtcgtactgctatatacattcgctaccgctaacgccgatacactatgcatagggtatcacgctaataatagtacagacacagtagacacagtactcgaaaaaaacgttacggttacacattccgttaatctgttagaggataagcataacggtaagctatgtaaactgagaggcgtagcaccattgcatttgggtaagtgtaatatagccggatggatactaggtaatcccgaatgcgaatcactatcaactgcaagttcatggtcttatatagtcgaaactagttcaagcgataacggtacatgttatcccggagactttatcgattacgaagagttgagagagcaattgtctagcgtaagctcattcgaaagattcgaaatttttccgaaaactagttcatggcctaatcacgattcaaataagggggtaacagccgcatgcccacacgcaggcgctaagtcattctataaaaatctgatatggctagtgaaaaaagggaattcttatccgaaactatcaaaatcatatattaacgataagggtaaggaggtactcgtattgtgggggatacaccatccatcaactagcgcagaccaacaatctctgtatcagaatgccgacgcatacgtattcgtagggactagtaggtactctaaaaaatttaaacccgaaatcgctattagaccgaaagtgagagaccaggagggaagaatgaattactattggacactagtcgaaccaggcgataagattacattcgaagcgacagggaatctagtggtaccgagatacgcattcgcaatggagagaaacgccggatccggaattattattagcgatactcccgtacacgattgcaatacaacatgtcagacaccaaaaggggcaattaatactagcctaccatttcagaatatacacccaattacaatcggtaagtgtccaaaatacgttaagtctacgaaacttagattggcaacagggttgagaaacgtaccatcaatacagtctagagggttgttcggagcaatcgccggattcatagagggggggtggaccggtatggtcgacggatggtacggataccatcatcaaaacgaacaggggtccggatacgcagccgatctgaaatcaacacagaacgcaatcgacgaaattacgaataaagtgaatagcgtaatcgaaaaaatgaatactcagtttacagccgtaggtaaggaatttaatcatctcgaaaaaagaattgagaatctgaataaaaaggtagacgacgggtttctagacatttggacatataatgccgaactgttagtgttactcgaaaacgaaagaacattagactatcacgattctaacgttaagaatctatacgaaaaagtgagatcgcaattgaagaataacgcaaaagagatagggaatgggtgtttcgaattctaccataaatgcgataatacatgtatggaatccgtaaaaaacggtacatacgattatccgaaatatagcgaagaagcaaaactgaatagggaagagattgacggagttaagttggagtcaactaggatttaccagatactcgcaatttactctacagtcgcatcaagtctagtgttagtcgttagcttaggcgcaattagtttttggatgtgttcaaacggatcactgcaatgtaggatttgcata

SEQ ID NO: 13 - DNA, 1494 bp, Influenza A virus
atggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaagagcatcccagtgctgggaaggaccctaagaaaacaggaggacccatatatagaagaatagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactaagaagcagatactgggccataaggaccaggagtggaggaaataccaatcaacaaaaggcatccgcaggccagatcagtgtgcagcctacattctcagtgcagcgaaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagt

SEQ ID NO: 14 - DNA, 1494 bp, Unknown
  Deoptimized Influenza A virus
atggctagtcaggggacaaaacgatcatacgaacaaatggagacaggaggggaaagacaggacgcaaccgaaattagggctagcgtagggagaatgatagggggaatcggtaggttttatatacaaatgtgtacagaactcaaactatccgattatgacggaagactgatacagaattcaattacaatcgaaagaatggtgttgtctgcattcgacgaaagacgtaataagtatctcgaagagcatccaagcgcaggtaaggatccaaaaaaaaccggaggaccaatctatagacggatagacggtaagtggatgcgcgaactgatactgtatgacaaagaggagattaggagggtttggcgacaagcgaataatggcgaagacgcaaccgcaggactgacacacattatgatatggcatagtaatcttaacgacgctacatatcaacgaactagagcactcgttagaaccggtatggatcctagaatgtgctcacttatgcagggatcaacactccctagacgatccggcgcagccggagccgcagttaagggagtcggaacaatcgcaatggagttaatcagaatgataaagagagggattaacgatagaaatttttggagaggcgaaaacggtagacggactagagtcgcttacgaaagaatgtgcaatatccttaagggtaagtttcagaccgcagcacaaagggctatgatggatcaggttagagagtctagaaatcccggaaacgccgaaatcgaagacctaatctttctcgctagatccgctctaatccttaggggatccgttgcgcataagagttgcttacccgcatgcgtttacggactcgcagtcgctagcggacacgatttcgaacgcgaagggtatagtctcgtcggaatcgacccattcaaattactgcaaaatagtcaggtagtgagtcttatgagacctaacgagaatcccgcacataaatcgcaactcgtatggatggcatgccattccgcagcattcgaagaccttagggtgagtagtttcatacgcggaaaaaaagtgatacctaggggtaagcttagtactaggggggtgcaaatcgctagtaacgagaatgtcgagacaatggactctaatacactcgaactgagatctagatattgggcaatcagaacacgatccggagggaatacgaatcaacaaaaagcaagcgcaggacagattagcgtgcaacctacattctcagtgcaacggaatctgccattcgaaagagcaaccgttatggccgcattctcagggaataacgaagggcgaacatccgatatgcgaaccgaagtgattaggatgatggaatccgctaaacccgaagacctatcttttcagggaaggggggtgttcgaattgtcagacgaaaaagcgacaaatccgatagtgccatctttcgatatgtctaacgagggatcatattttttcggagataatgccgaagagtacgatagt

SEQ ID NO: 15 - DNA, 1407 bp, Influenza A virus
atgaatccaaaccaaaagataataaccattggttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtataagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacaatatattgagaacacaagagtctgaatgtgcatgtgtaaatggttcttgctttactgtaatgaccgatggaccaagtgatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaaggattttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaag

SEQ ID NO: 16 - DNA, 1407 bp, Unknown
  Deoptimized Influenza A virus
atgaatcctaaccaaaaaattataacaatcggatccgtttgtatgacaatcggtatggctaacctaatactgcaaatcggtaatattatatcgatttggatctcacatagtatacaattgggtaatcagaatcagatagagacatgcaatcaatccgttattacatacgaaaataatacttgggttaatcagacatacgttaacatatcgaatactaatttcgctgccggacaatccgtcgttagcgttaagttagccggtaatagttcactatgccccgttagcgggtgggctatatactctaaagacaattcgattagaatcggatctaagggcgacgtattcgtaatacgcgaaccattcataagttgtagtccattagagtgtagaactttttttctaacacaaggcgctctattgaacgataagcatagtaacggtacaattaaggatagatcaccttatagaacattgatgtcatgtcctatcggcgaagtgcctagtccatacaatagtagattcgaatccgtcgcatggtccgctagcgcatgtcacgacgggattaattggttgactatagggattagcggacccgataacggcgcagtcgctgtgcttaagtataacggtattattaccgacactataaagagttggcgaaataacatactgagaacacaggaatccgaatgcgcatgcgtaaacggttcatgttttaccgtaatgactgacggacctagcgacggacaagcgtcatataagatttttagaatcgaaaaaggtaagatagtgaaatctgtcgagatgaacgctccgaattatcattacgaagagtgtagttgttatcccgattctagcgaaattacatgcgtatgtagggacaattggcacgggtctaatcgaccatgggtgtcattcaatcagaacttagagtatcagatagggtatatatgctcagggatattcggcgataatcctagaccgaacgataaaaccggatcatgcggaccagtgtcatctaacggcgctaacggagtgaaagggtttagtttcaaatacggtaacggcgtatggatcggacgaactaagtctatatctagtaggaacggattcgaaatgatatgggacccaaacgggtggaccggtaccgataataacttttcaatcaaacaggacatagtcggaattaacgaatggtccgggtatagcggatcattcgtgcaacatccagagttaaccggactcgattgcataagaccatgtttttgggtcgaattgattagggggagaccaaaagagaatactatatggactagcggatctagtattagcttttgcggagtgaatagcgataccgtagggtggtcatggccagacggagccgaactaccatttacaatcgataag

SEQ ID NO: 17 - DNA, 2274 bp, Influenza A virus
atggatgtcaacccgactctacttttcctaaaggttccagcgcaaaatgccataagcaccacattcccttatactggagatcctccatacagccatggaacaggaacagggtacaccatggacacagtcaacagaacacaccaatattcagaaaaggggaagtggacgacaaatacagaaactggggcaccccaactcaacccaattgatggaccactacctgaggataatgagccaagtggatatgcacaaacagactgtgtcctggaggctatggccttccttgaagaatcccacccagggatctttgagaactcatgccttgaaacaatggaagtcgttcaacaaacaagggtggacaaactaactcaaggtcgccaaacttatgattggacattaaacagaaatcaaccggcagcaactgcattagccaacaccatagaagtttttagatcgaatggtctaacagctaatgaatcaggaagactaatagattttctcaaggatgtgatggaatcaatggataaagaggaaatggagataacaacacactttcaaagaaaaaggagagtaagagacaacatgaccaaaaaaatggtcacacaaagaacaatagggaagaaaaaacaaaaagtgaataagagaggctatctaataagagctttgacattgaacacgatgaccaaagatgcagagagaggtaaattaaaaagaagggctattgcaacacccgggatgcaaattagagggttcgtgtactttgttgaaactatagctagaagcatttgcgagaagcttgaacagtctggacttccggttgggggtaatgagaagaaggccaaactggcaaatgttgtgagaaaaatgatgactaattcacaagacacagagctttctttcacaatcacaggggacaacactaagtggaatgaaaatcaaaaccctcgaatgtttttggcgatgattacatatatcacaaaaaatcaacctgagtggttcagaaacatcctgagcatcgcaccaataatgttctcaaacaaaatggcaagactaggaaaaggatacatgttcgagagtaagagaatgaagctccgaacacaaatacccgcagaaatgctagcaagcatcgacctgaagtatttcaatgaatcaacaaggaagaaaattgagaaaataaggcctcttctaatagatggcacagcatcattgagccctggaatgatgatgggcatgttcaacatgctaagtacggttttaggagtctcgatactgaatcttgggcaaaagaaatacaccaagacaacatactggtgggatgggctccaatcctccgacgattttgccctcatagtgaatgcaccaaatcatgagggaatacaagcaggagtggatagattctacaggacctgcaagttggtgggaatcaacatgagcaaaaagaagtcctatataaataaaacagggacatttgaattcacaagctttttttatcgctatggatttgtggctaattttagcatggagctgcctagttttggagtgtctggaataaatgagtcagctgatatgagcattggagtaacagtgataaagaacaacatgataaacaatgaccttggaccagcaacagcccagatggctcttcaattgttcatcaaagactacagatatacatataggtgccatagaggagacacacaaattcagacgagaagatcattcgagctaaagaagctatgggatcaaacccaatcaagggcaggactgttggtgtcagatgggggaccaaacttatacaatatccggaatcttcacatccctgaagtctgcttaaagtgggagctaatggatgaggattatcggggaagactttgtaatcccctaaatccctttgtcagccataaagaaattgagtctgtaaacaatgctgtagtgatgccagcccatggtccagccaaaagtatggaatatgatgccgttgcaactacacactcctggattcccaagaggaaccgctctattctcaacacaagccaaaggggaattcttgaggatgaacagatgtaccagaagtgctgcaacttgttcgagaaatttttccctagtagttcatatagaagaccagttggaatttctagcatggtggaggccatggtgtctagggcccggattgatgccagaattgacttcgagtctggacggattaagaaggaagagttctctgagatcatgaagatctgttccaccattgaagaactcagacggcaaaaataa

SEQ ID NO: 18 - DNA, 2274 bp, Unknown
  Deoptimized Influenza A virus
atggacgttaatccgacactattgtttctgaaagtgccagcccaaaacgctatatcgacaacattcccatacactggcgatccaccatactctcacggaaccggaacagggtacacaatggatacagtgaatagaacacaccaatatagcgaaaagggtaagtggacaactaacacagagacaggcgcacctcaattgaaccctatagacggacctctacctgaggataacgaacctagcggatacgctcaaaccgattgcgtactcgaggcaatggcatttctcgaagagtcacacccagggatattcgagaatagttgcttagagactatggaggtcgtgcaacagactagagtcgataagctaacacaggggagacagacatacgattggacacttaataggaatcagcctgccgcaaccgcactcgctaatacaatcgaagtgtttagatcgaacggactaaccgctaacgaatccggacgactaatcgatttccttaaagacgttatggagtcaatggataaagaggaaatggagataacaacacatttccaaagaaagagacgggttagggataatatgacaaaaaaaatggtgacacaacggacaatcggaaaaaaaaaacaaaaagttaataagagagggtatctgattagggccctaacactgaatacaatgacaaaagacgccgaacgcggtaagcttaagagacgcgcaatcgcaacacccggtatgcaaattagggggttcgtttatttcgtagagacaatcgctagatctatttgcgaaaaactcgaacaatccggactaccagtcgggggaaacgagaaaaaggctaagttagcgaacgtagtgagaaaaatgatgactaatagccaagataccgaacttagctttacgattaccggagataatacgaaatggaacgagaatcaaaaccctagaatgtttctcgcaatgataacatacataacaaagaatcaacccgaatggtttagaaacatactgtcaatcgcaccaattatgtttagcaataagatggccagattgggtaaggggtatatgttcgaatctaagagaatgaagcttagaacgcaaattcccgccgaaatgcttgcctcaatcgatcttaagtattttaacgagtcaactagaaaaaaaatcgaaaagattagaccactattgatagacggaaccgcaagcttatcacccggaatgatgatgggtatgttcaatatgcttagtacagtgctcggagtgagtatacttaacttagggcaaaaaaagtatacaaagactacatattggtgggacggactgcaatctagcgacgatttcgcactaatcgttaacgcacctaatcacgaggggattcaagccggagtcgataggttttatagaacatgtaagttagtcggaattaatatgtctaaaaaaaagtcatacataaacaaaaccggaacattcgaatttactagttttttttataggtacggattcgttgcgaatttctcaatggagttgcctagtttcggagtgagcggaataaacgaatccgccgatatgtcaatcggagtgacagtgattaagaataatatgattaataacgatctagggccagcaaccgcacaaatggcattgcaattgttcataaaagactatagatatacatatagatgccataggggcgatacacaaattcaaactagacggtcattcgagcttaaaaagttgtgggatcagacacaatctagagccggactgttagtgagcgacggggggcctaacctatacaacattagaaacctacatatacccgaagtgtgtcttaagtgggagcttatggacgaggattatagggggagactatgcaatccactaaacccattcgttagccataaagagatagagtccgttaataacgccgtagtgatgccagcacacggacccgctaagtctatggagtacgatgccgtcgcaacgacacatagttggataccgaaacggaatagatctatactgaatactagccaacgcggaatactcgaagacgaacaaatgtatcaaaagtgttgcaatctattcgaaaagttttttccgtcaagctcatataggagaccagtcggaattagctctatggtcgaggcaatggtgagtagggctaggattgacgctagaatcgatttcgaatccggacggattaaaaaagaggagttttccgaaattatgaagatatgctcaacaatcgaagagcttagacgacaaaagtaa

SEQ ID NO: 19 - DNA, 1698 bp, Influenza A virus
atgaaagtaaaactactgatcctgttatgtacatttacagctacatatgcagacacaatatgtataggctaccatgccaacaactcaaccgacactgttgacacagtacttgagaagaatgtgacagtgacacactctgtcaacctacttgaggacagtcacaatggaaaactgtgcctactaaaaggaatagcccccctacaattgggtaattgcagcgttgccggatggatcttaggaaacccagaatgcgaattactgatttccaaggaatcatggtcctacattgtagaaacaccaaatcctgagaatggagcatgttacccagggtatttcgccgactatgaggagctaagggagcaattgagttcagtatcttcatttgagagattcgaaatattccccaaagaaagctcatggcccaaccacaccgtaaccggagtatcagcatcatgctcccataatgggaaaagcagtttttacaaaaatttgctatggctgacggggaagaatggtttgtacccaaacctgagcaagtcctatgcaaacaacaaagagaaagaagtccttatactatggggtgttcatcacccgcctaacataggggaccaaaggaccctctatcacacagaaaatgcttatgtctctgtagtgtcttcacattatagcagaagattcaccccagaaataaccaaaaggcccaaagtaagagatcaggaaggaagaatcaactactactggactctgctggaacccggggatacaataatatttgaggcaaatggaaatctaatagcgccatggtatgctttcgcactgagtagaggctttggatcaggaatcatcacctcaaatgcaccaatggatgaatgtgatgctaagtgtcaaacacctcagggagctataaacagcagtcttcctttccagaatgtacacccagtcacaataggagagtgtccaaagtatgtcaggagtgcaaaattaaggatggttacaggactaaggaacatcccatccattcaatccagaggtttgtttggagccattgccggtttcattgaaggggggtggactggaatggtagatgggtggtatggttatcatcatcagaatgagcaaggatctggctatgctgcagatcaaaaaagcacacaaaatgccattaacgggattacaaacaaggtgaattctgtaattgagaaaatgaacactcaattcacagctgtgggcaaagaattcaacaaattggaaagaaggatggaaaacttaaataaaaaggttgatgatgggtttctagacatttggacatataatgcagaattgttggttctactggaaaatgaaaggactttggatttccacgactccaatgtgaagaatctgtacgagaaagtaaaaagccaattaaagaataatgccaaagaaataggaaatgggtgttttgaattctatcacaagtgtaacaatgaatgcatggagagtgtgaaaaatggaacttatgactatccaaaatattccgaagaatcaaagttaaacagggaaaaaattgatggagtgaaattggactcaatgggggtctatcagattctggcgatctactcaactgtcgccagttccctggttcttttggtctccctgggggcaatcagcttctggatgtgttccaatgggtctttgcagtgtagaatatgcatctga

SEQ ID NO: 20 - DNA, 1698 bp, Unknown
  Deoptimized Influenza A virus
atgaaagtgaaactgttaatactgttgtgcacttttaccgctacatacgccgatacaatttgcatagggtatcacgctaataatagtaccgatacagtcgacactgtgttggaaaagaacgtaaccgttacacactccgttaatctgttagaggattcccataacggtaagttgtgtctgttgaaagggatcgcaccattgcaattgggtaattgtagcgtagccggatggatattggggaatcccgaatgcgaactattgattagtaaagagtcatggtcatatatagtcgagacacctaatcccgaaaacggagcatgctatcccggatatttcgccgattacgaagagcttagagagcaattgtctagcgtaagctcattcgaaagattcgaaatttttccaaaagagtcaagttggcctaatcataccgtaacaggcgtatccgcatcatgtagtcataacggtaagtcaagcttttataagaatctgttatggttaaccggtaaaaacggactgtatccaaatctatctaagtcatacgcaaataataaagagaaagaggtactgattctatggggggtgcatcacccacctaatataggcgatcaaagaacattgtatcataccgaaaacgcatacgtatccgtcgttagctcacactatagtagaaggtttacacccgaaattactaagagacctaaggtaagggatcaggagggtaggattaattattattggactctacttgaaccaggcgatactatcatattcgaagctaacggaaatctaatcgcaccatggtacgcattcgcactatctagggggttcggatccgggattattacttctaacgctccaatggacgaatgcgacgcaaagtgtcagacaccacagggagcgattaatagttccctaccattccaaaacgtacaccccgttacaatcggcgaatgtccgaaatacgttagatccgctaaacttagaatggtgaccggactgagaaatataccatcaatccaatctagggggctattcggagccatagccggatttatcgaaggggggtggacagggatggtcgacggatggtatgggtatcaccaccaaaacgaacagggatccggatacgccgccgatcagaaatccacacaaaacgctattaacggaattacgaataaagtgaatagcgtaatcgaaaaaatgaatacacaatttactgccgtaggtaaggaattcaataagttagagagaaggatggagaatctgaataaaaaagtcgacgacggattcctagacatatggacatataacgccgaactgttagtgttgcttgagaacgaaaggacactagactttcacgattcaaacgttaaaaatctatacgaaaaagtcaaatcccaattgaaaaataacgctaaagagatagggaatgggtgtttcgaattctatcataagtgtaataacgaatgtatggaatccgttaaaaacggaacatacgattatccaaagtatagcgaagagtcaaaactgaatagggaaaaaatcgacggagtcaaacttgactcaatgggggtgtatcagatactcgcaatctatagtacagtcgcatctagcctagtactgttagtgagtctgggagcgataagcttttggatgtgttctaacggatcactgcaatgtaggatatgcatatga

SEQ ID NO: 21 - DNA, 1497 bp, Influenza A virus
atggcgtcccaaggcaccaaacggtcttatgaacagatggaaactgatggggatcgccagaatgcaactgagattagggcatccgtcgggaagatgattgatggaattgggagattctacatccagatgtgcactgaacttaaactcagtgattatgaagggcggttgatccagaacagcttgacaatagagaaaatggtgctctctgcttttgatgagagaaggaatagatatctggaagaacaccccagcgcggggaaagatcctaagaaaactggagggcccatatacaggagagtagatggaaaatggatgagggaacttgtcctttatgacaaagaagaaataaggcggatctggcgccaagccaacaatggtgaggatgcaacagctggtctaactcacatgatgatctggcattccaatttgaatgatacaacataccagagaacaagagctcttgttcgaaccggaatggatcccagaatgtgctctctgatgcagggctcgactctccctagaaggtccggagctgcaggtgctgcagtcaaaggaatcgggacaatggtgatggagctgatcagaatggtcaaacgggggatcaacgatcgaaatttctggagaggtgagaatgggcggaaaacaagaagtgcttatgagagaatgtgcaacattctcaaaggaaaatttcaaacagctgcacaaagagcaatggtggatcaagtgagagaaagtcggaacccaggaaatgctgagatcgaagatctcatatttctggcaagatctgcattgatattgagagggtcagttgctcacaaatcttgtctacctgcctgtgtgtatgggcctgcagtatccagtgggtacgatttcgaaaaagagggatattccttggtgggaatagaccctttcaaactacttcaaaatagccaagtatacagcctaatcagacctaacgagaatccagcacacaagagtcagctggtgtggatggcatgccattctgctgcatttgaagatttaagattgttaagcttcatcagagggaccaaagtatctccgcgggggaaactttcaactagaggagtacaaattgcttcaaatgagaacatggataatatgggatcgagtactcttgaactgagaagcgggtactgggccataaggaccaggagtggaggaaacactaatcaacagagggcctccgcaggccaaatcagtgtgcaacctacgttttctgtacaaagaaatctcccatttgaaaagtcaaccgtcatggcagcattcactggaaatacggagggaagaacctcagacatgagggcagaaatcataagaatgatggaaggtgcaaaaccagaagaagtgtcgttccgggggaggggagttttcgagctctcagatgagaaggcaacgaacccgatcgtgccctcttttgacatgagtaatgaaggatcttatttcttcggagacaatgcagaagagtacgacaattaa

SEQ ID NO: 22 - DNA, 1497 bp, Unknown
  Deoptimized Influenza A virus
atggctagtcagggtacgaaacggtcatacgaacagatggagactgacggagatagacaaaacgcaaccgaaattagggctagcgtcggtaagatgatcgacggaatcggacggttttatatacagatgtgtaccgaacttaagttgtccgattacgaagggagattgatccaaaattcgcttacaatcgaaaaaatggtgttaagcgcattcgacgaaagacggaataggtatctcgaagagcaccctagcgcaggtaaggatccgaaaaaaacaggggggccaatctatagacgggtcgacggaaagtggatgagagagctcgtactatacgataaagaggagataagacggatatggagacaggctaataacggcgaagacgcaaccgcagggttaacacatatgatgatttggcactctaatcttaacgatactacttatcaacggactagggcactcgttagaaccggaatggatcctagaatgtgctcacttatgcaggggtctacactccctagacgatccggagccgcaggcgcagccgttaagggaatcggaactatggttatggagttgattagaatggtgaaaagggggattaacgataggaatttttggagaggcgaaaacggtagaaaaactagatccgcatacgagagaatgtgcaatatactgaaagggaaattccaaaccgctgcgcaacgggctatggtcgatcaggtacgcgaatctagaaatcccggtaatgcggaaatcgaagatctgatattcctcgctagatccgcactgatacttagggggtcagtcgcacataaaagttgcttgcctgcatgcgtatacggacccgcagtgtctagcggatacgatttcgaaaaagaggggtatagtctagtcggaatcgatccatttaaactgttgcagaattcgcaagtgtatagtctaatcagacctaacgaaaatcccgcacacaaatcgcaactcgtatggatggcatgtcactccgccgcattcgaggatcttagattgctatcttttattaggggaacgaaagtgagtcctagggggaaactgtcaactaggggggtgcaaatcgcatctaacgagaatatggataatatgggatctagtacactcgaacttagatccggatattgggcaatcagaactagatccggggggaatacgaatcagcaacgcgctagcgctggacaaatcagtgtgcaacctacattcagtgtgcaacggaatctgccattcgaaaaatctaccgtaatggccgcttttacaggaaatacagagggacgaactagcgatatgagagcagagataatcagaatgatggagggagcaaaacccgaagaggtaagttttagggggaggggggtgttcgaattgtcagacgaaaaagctactaatccgatagtgccatctttcgatatgtctaacgaagggtcatattttttcggagataacgctgaggaatacgataattaa

SEQ ID NO: 23 - DNA, 1410 bp, Influenza A virus
atgaatccaaatcaaaaaataataacgattggctctgtttctctcaccattgccacaatatgcttccttacgcaaattgccatcctggtaactactgtaacattgcatttcaagcaatatgaatgcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgcaacattactggatttgcacctttttctaaggacaattcgattcggctttccgctggtggggacatctgggttacaagagaaccttatgtgtcatgcgatcctgacaagtgttatcaatttgcccttggacagggaacaacactaaacaacgggcattcaaatgacacagtacatgataggaccccttataggaccctattgatgaatgagttgggtgttccatttcatttgggaaccaagcaagtgtgcatagcatggtccagctcaagttgtcacgatggaaaagcatggctgcatgtttgtgtaacgggggatgataaaaatgcaactgctagcttcatttacaatgggaggcttgtagatagtataggttcatggtccaaaaaaatcctcaggacccaggagtcggaatgcgtttgtatcaatggaacttgtacagtagtaatgactgatgggagtgcttcaggaaaagctgatactaaaatactattcattgaggaggggaaaatcgttcatactagcctattgtcagggagtgctcagcatgtcgaggagtgctcctgttatcctcgatatcctggtgtcagatgtgtctgcagagacaactggaaaggctccaataggcccatcgtagatataaatgtaaaggattatagcattgtttccagttatgtgtgctcaggacttgttggagacacacccagaaaaaacgacagctccagcagtagccattgcttggatcctaacaatgaggaaggtggtcatggagtgaaaggctgggcctttgatgatggaaatgacgtgtggatgggaagaacgatcagcgagaagttacgctcaggatatgaaaccttcaaagtcattgaaggctggtccaaacctaactccaaactgcagataaataggcaagtcatagttgacagagataataggtccggttattctggtattttctctgttgaaggcaaaagctgcatcaatcggtgcttttatgtggagttgataaggggaaggaaccaggaaactgaagtcttgtggacctcaaacagtattgttgtgttttgtggcacctcaggtacatatggaacaggctcatggcctgatggggcggacatcaatctcatgcctatataa

SEQ ID NO: 24 - DNA, 1410 bp, Unknown
  Deoptimized Influenza A virus
atgaaccctaatcaaaaaataattacaatcggatccgttagtctgacaatcgctactatatgttttctgactcagatagcgatactcgttacaaccgttacattgcatttcaaacaatacgaatgcaattccccccctaacaatcaggtaatgttgtgcgaacctacaataatcgaacggaatattaccgagatagtgtatctgactaatacgactatcgaaaaagagatatgcccaaaactagccgaatatcggaattggtcaaaaccgcaatgtaacataaccggattcgcaccattttcgaaagacaattcgattaggttgtccgccggaggcgatatttgggttacacgcgaaccttatgtgtcatgcgatcccgataaatgctatcaattcgcactcggacaggggactacccttaataacggacattctaacgataccgtacacgatagaactccatatcgaacattgctaatgaacgagttaggcgtaccattccatttgggcactaaacaggtatgtatcgcatggtctagctctagttgccatgacggtaaggcttggttgcatgtgtgcgttaccggcgacgataagaacgcaaccgctagctttatatataacggtaggttggtcgactcaatcgggtcatggtcaaaaaaaatacttagaacgcaagagtccgaatgcgtatgcataaacggtacatgcaccgtagtgatgaccgacggatccgctagcggtaaggccgatacgaaaatactgtttatcgaagagggtaagatagtgcatacgagtctactatccggatccgctcaacatgtcgaagagtgttcatgttatcctaggtatcccggcgttagatgcgtatgtagggataattggaaagggagtaatagacctatagtcgatattaacgttaaggattattcaatcgtaagtagttatgtgtgtagcggactcgtaggcgatacacctagaaaaaacgatagctctagtagctcacattgcctagaccctaataacgaagagggggggcatggcgttaagggatgggcattcgacgacggtaacgacgtttggatgggtaggactattagcgaaaagcttagatccgggtatgagacattcaaagtgatagagggatggtctaaacctaattcaaaactgcaaattaataggcaagtgatagtcgatagggataatagatccgggtattccggaatttttagcgttgagggtaagtcatgtattaataggtgtttttatgtcgagcttattagggggagaaatcaggaaaccgaagtgttgtggacatccaattcaatcgtcgttttttgcggaactagcggaacatacggtaccggatcatggcccgacggagccgatattaaccttatgcctatataa

SEQ ID NO: 25 - DNA, 2274 bp, Influenza A virus
atggatgtcaatccgaccttacttttcttgaaagttccagcgcaaaatgccataagtactacattcccttatactggagatcctccatacagccatggaacaggaacaggatacaccatggacacagtcaacagaacacatcaatattcagaaaaggggaagtggacaacaaacacggaaactggagcgccccaacttaacccaattgatggaccactacctgaggacaatgaaccaagtggatatgcacaaacagactgcgtcctggaagcaatggctttccttgaagaatcccacccaggaatctttgaaaactcgtgccttgaaacgatggaagttattcaacaaacaagagtggacaaactgacccaaggtcgtcagacctatgattggacattgaacagaaatcagccggctgcaactgcgctagccaacactatagaggtcttcagatcgaatggtctgacagctaatgaatcgggaaggctaatagatttcctcaaggatgtgatagaatcaatggataaagaggagatggaaataacaacacacttccaaagaaaaagaagagtaagagacaacatgaccaagaaaatggtcacacaacgaacaataggaaagaagaagcaaagattggacaagagaagctatctaataagagcactgacattgaacacaatgactaaagatgcagagagaggtaaattaaagagaagagcaattgcaacacccggtatgcagatcagagggttcgtgtactttgtcgaaacactagctagaagtatttgtgagaagcttgaacagtctgggcttccggttggaggtaatgaaaagaaggctaaactggcaaatgttgtgagaaaaatgatgactaattcacaagacacagagctctctttcacaattactggagacaataccaaatggaatgagaatcaaaatcctcggatgttcctggcgatgataacatacatcacaagaaatcaacctgaatggtttagaaacgtcctgagcatcgcacctataatgttctcaaataaaatggcaagactagggaaaggatacatgttcgaaagcaagagcatgaagctccgaacacaaataccagcagaaatgctagcaagtattgacctgaaatactttaatgaatcaacaaaaaagaaaatcgagaaaataaggcctctcctaatagatggcacagtctcattgagtcctggaatgatgatgggcatgttcaacatgctaagtacagtcttaggagtctcaatcctgaatcttggacaaaagaagtacaccaaaacaacatactggtgggacggactccaatcctctgatgacttcgccctcatagtgaatgcaccaaatcatgagggaatacaagcaggggtggatagattctacagaacctgcaagctagtcggaatcaatatgagcaaaaagaagtcctacataaataggacagggacatttgaattcacaagctttttctatcgctatggatttgtagccaattttagcatggagctgcccagctttggggtgtctggaattaatgaatcggctgatatgagcattggggtaacagtgataaagaacaacatgataaacaatgaccttgggccagcaacagcccaaatggctcttcaactattcatcaaagactacagatatacgtaccggtgccacagaggagacacacaaattcagacaaggagatcattcgagctaaagaagctgtgggagcaaacccgctcaaaggcaggacttttggtttcggatggaggaccaaacttatacaatatccggaatctccacattccagaagtctgcttgaagtgggagctaatggatgaagactatcaggggaggctttgtaatcccctgaatccatttgtcagtcataaggagattgagtctataaacaatgctgtggtaatgccagctcacggtccagccaagagcatggaatatgatgctgttgctactacacactcctggatccctaagaggaaccgctccattctcaacacaagccaaaggggaattcttgaggatgaacagatgtatcagaagtgttgcaatctattcgagaaattcttccctagcagttcgtacaggagaccagttggaatttccagcatggtggaggccatggtgtctagggcccggattgatgcacggattgacttcgagtctggacggattaagaaagaggagttcgctgagatcatgaagatctgttccaccattgaagagctcagacggcaaaaatag

SEQ ID NO: 26 - DNA, 2274 bp, Unknown
  Deoptimized Influenza A virus
atggacgttaatccgacactactattccttaaggtgccagcgcaaaacgctatatcgacaacattcccatacaccggagatccaccatactcacacggaaccggaaccggatacactatggacacagtgaataggacacaccaatactccgaaaagggtaagtggacaactaataccgaaaccggagccccacaattgaatccgatagacggaccactaccagaggataacgaacctagcggatacgctcaaaccgattgcgtactcgaggcaatggcattccttgaggagtctcaccctgggatattcgaaaactcatgcttggagacaatggaagtgatacaacagactagagtcgataagttgacacaggggagacagacatacgattggacactgaataggaatcaacctgccgcaaccgcactcgctaatacaatcgaagtgttcagatctaacggacttacagctaacgaaagcggacgactaatcgatttccttaaggacgttatcgaatcaatggataaggaggaaatggagattacgacacactttcaacgtaaacggagagtgagagacaatatgacaaaaaagatggtcacacaacggacaatcggtaagaaaaagcagagactcgataagagatcatatctgatacgcgcactgacacttaatacaatgacaaaggacgccgaacgcggtaagcttaagagacgcgcaatagcgacacccggtatgcaaattagggggttcgtatatttcgtcgagacactcgctagatcgatttgcgaaaagctagagcaatccggactgccagtcggcggaaacgaaaaaaaggcaaaactcgcaaacgtcgttaggaaaatgatgactaattcccaagacacagagttaagctttacaattaccggagataacacaaaatggaacgagaatcagaatcctagaatgtttctcgcaatgataacatacattactagaaaccaacccgaatggtttagaaacgtactatcaatcgcaccaattatgtttagcaataagatggctagattgggtaaggggtatatgttcgaatccaaatctatgaagcttagaacacagatacctgccgaaatgctcgctagtatcgatcttaagtattttaacgaatcgacaaaaaaaaagatagagaaaattagaccactattgatcgacggaacggttagcctatcccccggaatgatgatgggaatgttcaatatgctatcgacagtgttaggcgttagcatactgaatctcggacagaagaaatacactaagactacatattggtgggacggactgcaatctagcgacgatttcgcacttatcgttaacgctcctaatcacgaagggatacaagccggagtcgatagattctataggacatgtaagttagtcggaattaatatgagtaagaaaaaatcatacattaatagaaccggaacattcgaattcacaagctttttttacagatacggattcgtcgctaattttagtatggagctacctagcttcggagtgagcggaattaacgaatccgccgatatgtcaatcggagtgacagtcattaagaataatatgattaataacgatctagggccagcaaccgctcaaatggccctacaattgttcattaaggactataggtatacatatagatgtcataggggggatacgcaaattcaaactagacgatcattcgaactgaaaaaattgtgggagcaaactagatcgaaagccggactgttagtgagcgacggggggcctaatctgtataacatacggaatctgcatatacccgaagtgtgtcttaagtgggagcttatggacgaggattaccaaggtaggctatgcaatccactgaatccattcgtaagccataaagagatagagtctattaataacgcagtcgttatgcctgcacacggaccagcgaaatctatggagtacgacgcagtcgcaacaacacatagttggataccgaaacggaatagatcgatactgaatacaagtcaaagggggatactcgaagacgaacagatgtaccaaaagtgttgcaatctattcgagaaatttttccctagctctagctatagacggccagtcggaattagtagtatggtcgaggctatggtgagtagagcgagaatcgacgctagaatcgatttcgaatccggacggattaagaaagaggaattcgcagagataatgaagatttgctcaacaatcgaagagcttagacggcaaaagtag

SEQ ID NO: 27 - DNA, 1689 bp, Influenza A virus
atggccatcatttatctcattctcctgttcacagcagtgagaggggaccagatatgcattggataccatgccaataattccacagagaaggtcgacacaattctagagcggaacgtcactgtgactcatgccaaggacattcttgagaagacccataacggaaagttatgcaaactaaacggaatccctccacttgaactaggggactgtagcattgccggatggctccttggaaatccagaatgtgataggcttctaagtgtgccagaatggtcctatataatggagaaagaaaacccgagagacggtttgtgttatccaggcagcttcaatgattatgaagaattgaaacatctcctcagcagcgtgaaacatttcgagaaagtaaagattctgcccaaagatagatggacacagcatacaacaactggaggttcacgggcctgcgcggtgtctggtaatccatcattcttcaggaacatggtctggctgacaaagaaaggatcaaattatccggttgccaaaggatcgtacaacaatacaagcggagaacaaatgctaataatttggggggtgcaccatcccaatgatgagacagaacaaagaacattgtaccagaatgtgggaacctatgtttccgtaggcacaccaacattgaacaaaaggtcaaccccagacatagcaacaaggcctaaagtgaatggacaaggaggtagaatggaattctcttggaccctattggatatgtgggacaccataaattttgagagtactggtaatctaattgcaccagagtatggattcaaaatatcgaaaagaggtagttcagggatcatgaaaacagaaggaacacttgggaactgtgagaccaaatgccaaactcctttgggagcaataaatacaacattgccttttcacaatgtccacccactgacaataggtgagtgccccaaatatgtaaaatcggagaagttggtcttagcaacaggactaaggaatgttccccagattgaatcaagaggattgtttggggcaatagctggttttatagaaggaggatggcaaggaatggttgatggttggtatggataccatcacagcaatgaccagggatcagggtatgcagcagacaaagaatccactcaaaaggcatttgatggaatcaccaacaaggtaaattctgtgattgaaaagatgaatacccaatttgaagctgttgggaaagaattcagtaacttagagagaagactggagaacttgaacaaaaagatggaagacgggtttctagatgtgtggacatacaatgctgagcttctagttctgatggaaaatgagaggacacttgactttcatgattctaatgtcaagaatctgtatgataaagtcagaatgcagctgagagacaacgtcaaagaactaggaaatggatgttttgaattttatcacaaatgtgatgatgaatgcatgaatagtgtgaaaaacgggacgtatgattatcccaagtatgaagaagagtctaaactaaatagaaatgaaatcaaaggggtaaaattgagcagcatgggggtttatcaaatccttgccatttatgctacagtagcaggttctctgtcactggcaatcatgatggctgggatctctttctggatgtgctccaacgggtctctgcagtgcaggatctgcatatga

SEQ ID NO: 28 - DNA, 1689 bp, Unknown
  Deoptimized Influenza A virus
atggcaataatctatctgatactgttgtttacagccgttaggggcgatcagatatgcatagggtatcacgctaataatagtaccgaaaaagtcgatacaatactcgaaagaaacgtaaccgttacacacgctaaagatatactcgaaaagacacataacggtaagctatgcaaacttaacggtataccaccacttgagttaggcgattgctcaatcgcaggatggttgttggggaatcccgaatgcgataggctattgagcgtacccgaatggtcttatattatggaaaaagagaatcctagagacggattgtgttatcccggatcttttaacgattacgaagagcttaaacatctgctatctagcgttaaacatttcgaaaaagtgaaaattctgccaaaagataggtggacacagcatacgactaccggaggatctagggcatgcgccgttagcggtaatccgtcattctttagaaatatggtatggttgacaaaaaaggggtctaattatccagtcgctaagggatcgtataataatacaagcggagagcaaatgttgattatatggggagtgcatcaccctaacgacgaaaccgaacaacggacactgtatcaaaacgtcggaacatacgttagcgtcggtacaccaactctgaataaaagatcgactcccgatatcgcaactagaccaaaagtgaacggacagggggggagaatggagtttagttggacactactcgatatgtgggatacaattaatttcgaatcaaccggtaatctgatcgcacccgaatacgggtttaagattagtaaaagggggtcatccggtattatgaaaaccgaaggtacactagggaattgcgaaactaagtgtcagacaccactaggggctattaatacaacactaccatttcataatgtgcatccattgacaatcggagagtgtcctaagtatgtgaaatccgaaaaactagtgcttgcaaccggactgagaaacgtaccgcaaatcgaatccagagggttgttcggagcaatcgcagggtttatcgaaggggggtggcagggaatggtcgacggatggtatgggtatcatcactctaacgatcagggatccggatacgcagccgataaggagtcaacccaaaaagcattcgacggaattactaataaggtgaatagcgtaatcgaaaaaatgaatacacaattcgaagccgtcggtaaagagttttcgaatctcgaaaggagacttgagaatctgaataaaaaaatggaggacggattcttagacgtatggacatataatgccgaactgttagtccttatggagaacgaacggacactagactttcacgatagtaacgttaagaatctgtatgacaaagtgagaatgcaattgagagacaatgtgaaagagctaggtaacggatgtttcgaattctatcataaatgcgacgacgagtgtatgaatagcgttaaaaacggtacatatgactatcctaagtatgaggaagagtcaaagcttaatagaaacgagattaagggagtgaaactatctagtatgggagtgtatcagatactcgcaatatacgctacagtcgccggatccctatcacttgcgattatgatggccggaattagcttttggatgtgctctaacggatcattgcaatgtaggatttgcatatga

SEQ ID NO: 29 - DNA, 1497 bp, Influenza A virus
atggcgtcccaaggcaccaaacggtcttatgaacagatggaaactgatggggaacgccagaatgcaactgaaatcagagcatccgtcgggaagatgattgatggaattggacgattctacatccaaatgtgcaccgaacttaaactcagtgattatgaggggcggctgatccagaacagcttaacaatagagagaatggtgctctctgcttttgacgagaggaggaataaatatctggaagaacatcccagcgcggggaaggatcctaagaaaactggaggacccatatacaagagagtagatggaaagtggatgagggaactcgtcctttatgacaaagaagaaataaggcgaatctggcgccaagctaataatggtgatgatgcaacagctggtctgactcacatgatgatctggcattccaatttgaatgatacaacataccagagaacaagagctcttgttcgcaccggaatggatcccaggatgtgctctttgatgcagggttcgactctccctaggaggtctggagccgcaggcgctgcagtcaaaggagttgggacaatggtgatggagttgatcaggatgatcaaacgtgggatcaatgatcggaacttctggagaggtgagaatgggcggaaaacaaggattgcttatgagagaatgtgcaacattctcaaaggaaaatttcaaacagctgcacaaagagcaatgatggatcaagtgagagaaagccggaacccaggaaatgctgagatcgaagatctcatctttctggcacggtctgcactcatattgagagggtcagttgctcacaaatcttgtctgcctgcctgtgtgtatggacctgccgtagccagtgggtacgacttcgaaaaagagggatactctttagtagggatagaccctttcaaattgcttcaaaacagccaagtatacagcctaatcagaccgaacgagaatccagcacacaagagtcagctggtgtggatggcatgcaattctgctgcatttgaagatctaagagtatcaagcttcatcagagggaccaaagtaatcccaagggggaaactttccactagaggagtacaaattgcttcaaatgaaaacatggatactatggaatcaagtactcttgaactgagaagcaggtactgggccataaggaccagaagtggaggaaacactaatcaacagagggcctctgcaggtcaaatcagtgtacaacctacgttttctgtgcaaagaaacctcccatttgacaaaccaaccatcatggcagcattcactgggaatgcagagggaagaacatcagacatgagggcagaaatcataaggatgatggaaggtgcaaaaccagaagaagtgtccttccaggggcggggagtcttcgagctctcggacgaaaaggcaacgaacccgatcgtgccctcttttgacatgagtaatgaaggatcttatttcttcggagacaatgcagaggagtacgacaattaa

SEQ ID NO: 30 - DNA, 1497 bp, Unknown
  Deoptimized Influenza A virus
atggctagtcaggggacaaaacggtcttacgaacaaatggagactgacggagaaagacagaacgcaaccgaaatcagggctagcgtaggtaagatgatcgacggaatcggtaggttctatatccaaatgtgtaccgaactgaaattgtccgattacgaagggagactgatacagaattcgcttacaatcgaacggatggtgttaagcgcattcgacgaaaggcgtaataagtatctcgaggaacaccctagcgcagggaaagaccctaaaaaaacagggggaccaatctataaaagagtcgacggtaagtggatgcgcgaactcgtactatacgataaagaagagattagacggatttggcgacaagcgaataacggagacgacgctaccgcagggttgacacatatgatgatatggcactctaatcttaacgatacgacatatcaacgaactagggcactcgttaggaccggaatggaccctagaatgtgttcacttatgcaggggtctacactccctagacggtcaggcgcagccggagccgcagttaagggagtcggaacaatggtaatggaattgataagaatgatcaaaagggggattaacgataggaatttttggagaggcgaaaacggtaggaaaactaggatcgcatacgaacggatgtgcaatatccttaagggaaaattccaaaccgcagcacaacgcgctatgatggatcaggttagagagtctaggaatcccggtaacgctgaaatcgaagatctgatattcctcgctagatccgcactcatacttagggggtcagtcgcacataagtcttgcttacccgcttgcgtatacggaccagcagtcgctagcggatacgatttcgaaaaagaggggtatagtctcgtagggatcgatccatttaaactgttgcaaaatagtcaggtgtatagtctgattagaccgaatgagaatcccgcacacaaatcgcaactcgtatggatggcatgcaattccgccgcattcgaagaccttagagtgagtagttttatcagagggactaaagtgatacctaggggaaaactatctactaggggagtgcaaatcgcatctaacgagaatatggatactatggagtctagtacactcgaactgagatctagatattgggcaatcagaactagatccggagggaatacgaatcagcaacgcgctagcgcagggcaaatctctgtgcaacctacatttagcgtgcaacggaatctgccattcgataagccaactattatggccgcatttaccggaaacgctgagggacggactagcgatatgagagccgaaatcataaggatgatggagggagctaaacccgaagaggtgtcatttcagggtaggggggtattcgaattgtccgacgaaaaagcgactaatccaatcgtaccgtctttcgatatgtctaacgagggatcatactttttcggagataacgccgaagagtacgataattaa

SEQ ID NO: 31 - DNA, 1410 bp, Influenza A virus
atgaatccaaatcaaaagataataacaattggctctgtctctctcaccattgcaacagtatgcttcatcatgcagattgccatcctggcaactactgtgacattgcattttaaacaacatgagtgcgactcccccgcgagcaaccaagtaatgccatgtgaaccaataataatagaaaggaacataacagagatagtgtatttgaataacaccaccatagagaaagagatttgccccgaagcagtggaatacagaaattggtcaaagccgcaatgtcaaattacaggatttgcacctttttctaaggacaattcaatccggctttctgctggtggggacatttgggtgacgagagaaccttatgtgtcatgcgatcctggcaagtgttatcaatttgcactcgggcaggggaccacactagacaacaaacattcaaatggcacaatacatgatagaatccctcaccgaaccctattaatgaatgagttgggtgttccatttcatttaggaaccaaacaagtgtgtgtagcatggtccagctcaagttgtcacgatggaaaagcatggttgcatgtttgtgtcactggggatgatagaaatgcgactgctagcttcatttatgacgggaggcttgtggacagtattggttcatggtctcaaaatatcctcaggacccaggagtcggaatgcgtttgtatcaatgggacttgcacagtagtaatgactgatggaagtgcatcaggaagagccgatactagaatactattcattaaagaggggaaaattgtccatattagcccattgtcaggaagtgctcagcatatagaggagtgttcctgttaccctcgatatcctgacgtcagatgtatctgcagagacaactggaaaggctctaataggcccgttatagacataaatatggaagattatagcattgattccagttatgtgtgctcagggcttgttggcgacacacccaggaacgacgacagctctagcaatagcaattgcagggatcctaacaatgagagagggaatccaggagtgaaaggctgggcctttgacaatggagatgatgtatggatgggaagaacaatcaacaaagattcacgctcaggttatgaaactttcaaagtcattggtggttggtccacacctaattccaaatcgcaggtcaatagacaggtcatagttgacaacaataattggtctggttactctggtattttctctgttgagggcaaaagctgcatcaataggtgcttttatgtggagttgataaggggaaggccacaggagactagagtatggtggacctcaaacagtattgttgtgttttgtggcacttcaggtacttatggaacaggctcatggcctgatggggcgaacatcaatttcatgcctatataa

SEQ ID NO: 32 - DNA, 1410 bp, Unknown
  Deoptimized Influenza A virus
atgaatcctaaccagaaaattattactatagggtcagtgtcattgactatcgcaaccgtatgctttattatgcaaatagcgatactcgcaactaccgtaacattgcattttaaacaacacgaatgcgatagtcccgctagcaatcaggtaatgccatgcgaacctattataatcgaacggaatattaccgagatagtgtatcttaacaatactactatcgaaaaagagatatgcccagaggccgtcgagtatagaaattggtctaaacctcaatgtcagattaccggattcgcaccattctctaaagacaattcgattagattgtccgccggaggcgatatatgggtgacacgcgaaccttatgtgtcatgcgatcccggtaagtgttatcaattcgcactcggacaggggactacactcgataataaacattctaacggtacgatacacgataggattccacataggacactattgatgaacgagttaggcgtaccgtttcatctaggcactaaacaggtatgcgttgcgtggtctagctcatcatgtcatgacggtaaggcatggttgcatgtgtgcgtaaccggcgacgatagaaacgctaccgctagttttatatacgacggtaggctagtcgattcaatcggatcatggtcacagaatatacttagaacacaggaatccgaatgcgtttgtattaacggtacatgtacagtcgttatgaccgacggatccgcatccggtagggccgatactaggatactgtttataaaagagggcaaaatcgtgcatattagcccacttagcggatccgcacaacatatcgaagagtgtagttgctatcctaggtatcctgacgttagatgtatttgcagagacaattggaaagggtctaatagacccgtaatcgatatcaatatggaggattattcaatcgatagctcttatgtgtgtagcggattagtcggcgatacacctagaaacgacgatagctctagtaattcgaattgtagggaccctaataacgagagaggcaatcccggcgttaaagggtgggcattcgataacggcgacgacgtttggatggggcgaacaattaataaggactctagatccgggtatgagacattcaaagtgataggggggtggtctacacctaactcaaaatctcaagtgaataggcaagtgatagtcgacaataacaattggtcagggtatagcggtatattctcagtcgagggtaagtcatgtattaatagatgtttttacgttgagttgattagggggcgaccacaagagactagagtgtggtggactagtaatagtatagtcgttttttgcggaactagcggtacatacggaaccggatcatggcctgacggagcgaatattaattttatgccaatctaa

SEQ ID NO: 33 - DNA, 2274 bp, Influenza A virus
atggatgtcaatccgactctactgttcctaaaggttccagcgcaaaatgccataagcaccacattcccttatactggagatcctccatacagccatggaacaggaacagggtacaccatggacacagtcaacagaacacaccaatattcagagaaggggaagtggacgacaaatacagaaactggggcaccccaactcaacccaattgatggaccactacctgaggataatgagccgagtggatatgcacaaacagattgtgtcctggaggctatggccttccttgaagaatcccacccaggtatctttgagaactcatgccttgaaacaatggaagtcgttcaacaaacaagggtggacaaactaacccaaggtcgccagacttatgattggacattaaacagaaatcaaccggcagcaactgcactagccaacaccatagaagtttttagatcgaatggactaacagctaatgaatcaggaaggctaatagatttcctcaaggatgtgatggaatcaatggataaagaggaaatggagataacaacacactttcaaagaaaaaggagagtaagagacaacatgaccaagaaaatggtcacacaaagaacaatagggaagaaaaaacaaagagtgaataagagaggctatctaataagagctttgacattgaacacgatgaccaaagatgcagagagaggtaaattaaaaagaagggctattgcaacaccagggatgcaaattagagggttcgtgtacttcgttgaaactttagctagaagcatttgcgaaaagcttgaacagtctggacttccggttgggggtaatgaaaagaaggccaaactggcaaatgttgtgagaaaaatgatgactaattcacaagacactgagctttctttcacaatcactggggacaacactaaatggaatgaaaatcaaaaccctcgaatgtttttggcgatgattacatatatcacaaaaaatcaacctgagtggttcagaaacatcctgagcatcgcaccaataatgttctcaaacaaaatggcaagactaggaaaaggatacatgttcgagagtaagagaatgaagctccgaacacaaatacccgcagaaatgctagcaagcattgacctgaagtatttcaatgaatcaacaaggaagaaaattgagaaaataaggcctcttctaatagatggcacagtatcattgagccctgggatgatgatgggcatgttcaacatgctaagtacggttttaggagtctcaatactgaatcttgggcaaaagaaatacaccaagacaacatactggtgggatgggctccaatcctccgacgattttgccctcatagtgaatgcaccaaatcatgagggaatacaagcaggagtggatagattctacaggacctgcaagttagtgggaatcaacatgagcaaaaagaagtcctatataaataaaacagggacatttgaattcacaagctttttttatcgatatggatttgtggctaattttagcatggagcttcccagttttggagtgtctggaataaacgagtcagctgatatgagcattggagtaacagtgataaagaacaacatgataaacaatgaccttggaccagcaacagcccagatggctctccaattgttcatcaaagactacagatatacatataggtgccatagaggagacacacaaattcagacgagaagatcattcgagctaaagaagctgtgggatcaaacccaatcaagggcaggactattggtatcagatgggggaccaaacttatacaatatccggaaccttcacatccctgaagtctgcttaaagtgggagctaatggatgagaattatcggggaagactttgtaaccccctgaatccctttgtcagccataaagaaattgagtctgtaaacaatgctgtagtgatgccagcccatggtccagccaaaagtatggaatatgatgccgttgcaactacacactcctggattcccaagaggaaccgctccattctcaacacaagccaaaggggaattcttgaggatgaacagatgtaccaaaagtgctgcaacttgttcgagaaatttttccctagtagttcatataggagaccgattggaatttctagcatggtggaggccatggtgtctagggcccggattgatgccagaattgacttcgagtctggacggattaagaaggaagagttctctgagatcatgaagatctgttccaccattgaagaactcagacggcaaaaataa

SEQ ID NO: 34 - DNA, 2274 bp, Unknown
  Deoptimized Influenza A virus
atggacgttaaccctacactactattccttaaggtgccagcccaaaacgcaattagcactacattcccatacacaggcgatccaccatactctcacggaaccggaaccggatacactatggatactgtgaatagaacacaccaatatagcgaaaagggtaagtggacaacgaatacagagacaggcgcaccacaattgaatccgatagacggacctctaccagaggataacgaacctagcggatacgctcaaaccgattgcgtactcgaggcaatggcattccttgaggaatcgcatccagggatattcgaaaatagttgcctagagactatggaggtcgtgcaacaaactagagtcgataagttgacacagggtaggcagacatacgattggacactgaatagaaaccaacctgccgcaaccgcactagcgaatacaatcgaagtgtttaggtctaacggactaaccgctaacgaatccggaagattgatcgatttccttaaggacgttatggagtcaatggataaagaggagatggagattactacacatttccaacgaaaaagacgcgttagggataatatgacaaaaaagatggtgacacaacggacaatcggaaaaaaaaagcaaagagtgaataagagggggtatctgattagagcccttacattgaatacaatgactaaagacgccgaaaggggtaagcttaagagacgcgctatcgcaacacccggtatgcaaattagggggttcgtatatttcgtcgagacactcgcaagatccatatgcgaaaaactcgagcaatccggactacccgtaggggggaacgaaaaaaaagctaagctcgcaaacgtcgtgagaaaaatgatgacaaactcacaggataccgaactgtcattcacaattaccggagataatactaagtggaacgagaatcaaaaccctagaatgtttctcgctatgattacatatattacgaaaaaccaacccgaatggtttagaaacatactatcaatcgcaccaattatgtttagcaataagatggctagactgggtaaggggtatatgttcgaatctaagagaatgaagcttagaacacaaattcctgccgaaatgttagcctcaatcgatcttaagtactttaacgagagtacacggaaaaaaatcgaaaagattagaccgttactgatagacggaaccgttagcctatcacccggaatgatgatggggatgtttaatatgctatctacagtgttaggcgtaagcatacttaacttagggcaaaaaaagtatacaaagactacatattggtgggacggactgcaatctagcgacgatttcgcattgatcgttaacgcacctaaccacgagggaatacaagccggagtcgatagattctatagaacatgtaagttagtcggaattaatatgagtaagaaaaagtcatacattaacaaaaccggaactttcgaatttacgagttttttttataggtacggattcgttgcgaattttagtatggagttaccgtcattcggagtgagcggaattaacgaatccgccgatatgtcaatcggagtgacagtgattaagaacaatatgattaataacgatctcggacccgcaaccgcacaaatggccttacaactattcataaaggattatagatatacatatagatgccataggggggatacacaaattcagacacgaagatcattcgaattgaaaaaactatgggatcaaacacaatccagagccggactactcgtaagcgatgggggacctaatctgtataacatacggaatctacacatacccgaagtgtgtcttaagtgggagcttatggacgaaaactatagggggagactatgcaatccacttaatccattcgttagccataaagagatagagtccgttaataacgccgtagtgatgccagcccacggaccagctaaatctatggagtacgacgcagtcgcaactacacatagttggataccgaaacggaatagatcaatactgaatacgtcacaaagggggatactcgaagacgaacagatgtatcaaaagtgttgcaatttgttcgaaaaattttttccgtctagctcatacagacgacctatagggataagctctatggtcgaggcaatggtgagtagggctaggatagacgctaggatcgatttcgaatccggacggattaaaaaagaggagtttagcgagattatgaagatttgctcaacaatcgaagagcttagaagacaaaaataa

SEQ ID NO: 35 - DNA, 1701 bp, Influenza A virus
atgaagactatcattgctttgagctacattctatgtctggttttcgctcaaaaacttcccggaaatgacaacagcacggcaacgctgtgccttgggcaccatgcagtaccaaacggaacgatagtgaaaacaatcacgaatgaccaaattgaagttactaatgctactgagctggttcagagttcctcaacaggtgaaatatgcgacagtcctcatcagatccttgatggagaaaactgcacactaatagatgctctattgggagaccctcagtgtgatggcttccaaaataagaaatgggacctttttgttgaacgcagcaaagcctacagcaactgttacccttatgatgtgccggattatgcctcccttaggtcactagttgcctcatccggcacactggagtttaacaatgaaagcttcaattggactggagtcactcaaaatggaacaagctctgcttgcaaaaggagatctaataacagtttctttagtagactgaattggttgacccacttaaaattcaaatacccagcattgaacgtgactatgccaaacaatgaaaaatttgacaaattgtacatttggggggttcaccacccgggtacggacaatgaccaaatcttcttgtatgctcaagcatcaggaagaatcacagtctctaccaaaagaagccaacaaactgtaatcccgaatatcggatccagacctagagtaaggratatccccagcagaataagcatctattggacaatagtaaaaccgggagacatacttttgattaacagcacagggaatctaattgctcctaggggttacttcaaaatacgaagtgggaaaagctcaataatgagatcagatgcacccattggcaaatgcaattctgaatgcatcactccaaatggaagcattcccaatgacaaaccatttcaaaatgtaaacagaatcacatatggggcctgtcccagatatgttaagcaaaacactctgaaattggcaacagggatgagaaatgtaccagagaaacaaactagaggcatatttggcgcaatcgcgggtttcatagaaaatggttgggagggaatggtggatggttggtacggtttcaggcatcaaaattctgagggaataggacaagcagcagatctcaaaagcactcaagcagcaatcaatcaaatcaatgggaagctgaataggttgatcgggaaaaccaacgagaaattccatcagattgaaaaagaattctcagaagtagaagggagaattcaggacctcgagaaatatgttgaggacactaaaatagatctctggtcatacaacgcggagcttcttgttgccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcataggatcaatcagaaatggaacttatgaccatgatgtatacagagatgaagcattaaacaaccggttccagatcaaaggcgttgagctgaagtcaggatacaaagattggatcctatggatttcctttgccatatcatgttttttgctttgtgttgttttgttggggttcatcatgtgggcctgccaaaaaggcaacattaggtgcaacatttgcatttga

SEQ ID NO: 36 - DNA, 1701 bp, Unknown
  Deoptimized Influenza A virus
atgaaaacaattatcgcactgtcatacatactgtgtctggtattcgctcaaaaattgcccggtaacgacaattcaaccgctacattgtgcttagggcatcacgccgtaccgaacggaactatcgttaagacaattactaacgaccaaatcgaagtgactaacgctacagagttggtgcaatcctctagtacaggcgaaatatgcgattcaccacaccaaatccttgacggagagaattgtacacttatcgacgcactattaggcgatccacaatgcgacggatttcagaataaaaaatgggatctattcgttgagagatccaaagcttattcaaattgttatccatacgacgtaccggattacgctagccttaggtcactcgttgcgtcaagcggtactctcgaattcaataacgagtcattcaattggactggcgttacgcaaaacggaactagtagcgcatgtaaaagacggtctaataatagcttttttagcagactgaattggttgactcatctgaaattcaaatatcccgcacttaacgttactatgcctaataacgaaaaattcgataagctatatatatggggcgtacaccatcccggaacggataacgatcagatattcttgtacgctcaagctagcggtaggattaccgttagtactaaaagatcccaacaaaccgtaattccgaatatcggatctagacctagggtgagaratataccgtctaggattagcatatattggactatcgttaaacccggagacatactgttgatcaatagtacaggcaatctgatcgcacctagggggtatttcaaaattagatccggtaagtctagcattatgagatccgacgcaccaatcggtaaatgtaatagcgaatgcattacaccaaacggatcaatccctaacgataagccattccaaaacgtaaataggattacatacggcgcatgccctagatacgttaaacagaatacgcttaaacttgcgacaggtatgcgaaacgtacccgaaaaacagactagggggatattcggcgcaatcgccggatttatcgaaaacggatgggagggtatggtcgacggatggtacggatttagacatcaaaatagcgaagggatagggcaagccgccgatctgaaatcaacgcaagccgctattaatcaaattaacggaaaactgaatagattgatcggtaagactaacgaaaaatttcaccaaatcgaaaaagagtttagcgaagttgagggaaggatacaagaccttgagaaatacgttgaggatactaagatcgacctatggtcatataatgccgagttgctagtcgcactcgagaatcagcatacaatcgatctgactgatagcgaaatgaataaattgttcgaaagaacgaaaaaacaattgcgcgaaaacgccgaagacatggggaatgggtgttttaagatataccataaatgcgataacgcatgcatagggtcaatcagaaacggaacatacgatcacgacgtatatagagacgaagcccttaataatagattccaaattaaaggcgttgagcttaaaagcggatacaaagactggatactgtggattagtttcgcaatctcatgctttctattgtgcgttgtgctattggggttcataatgtgggcatgtcagaaagggaatattagatgcaatatttgtatatga

SEQ ID NO: 37 - DNA, 1497 bp, Influenza A virus
atggcgtcccaaggcaccaaacggtcttatgaacagatggaaactgatggggatcgccagaatgcaactgagattagggcatccgtcgggaagatgattgatggaattgggagattctacatccaaatgtgcactgaacttaaactcagtgatcatgaagggcgattgatccagaacagcttgacaatagagaaaatggtgctctctgcttttgatgaaagaaggaataaatacctggaagaacaccccagcgcggggaaagatcccaagaaaactgggggacccatatacaggagagtagatggaaaatggatgagggaactcgtcctttatgacaaagaagaaataaggcgaatctggcgccaagccaacaatggtgaggatgcgacagctggtctaactcacataatgatctggcattccaatttgaatgatgcaacataccagaggacaagagctcttgttcgaactggaatggatcccagaatgtgctctctgatgcagggctcgactctccctagaaggtccggagcggcaggtgctgcagtcaaaggaatcgggacaatggtgatggaactgatcagaatggtcaaacgggggatcaacgatcgaaatttctggagaggtgagaatgggcggaaaacaagaagtgcttatgagagaatgtgcaacattcttaaaggaaaatttcaaacagctgcacaaagagcaatggtggatcaagtgagagaaagtcggaatccaggaaatgctgagatcgaagatctcatatttttggcaagatctgcattgatattgagagggtcagttgctcacaaatcttgcctacctgcctgtgcgtatggacctgcagtatccagtgggtacgacttcgaaaaagagggatattccttggtgggaatagaccctttcaaactacttcaaaatagccaaatatacagcctaatcagacctaacgagaatccagcacacaagagtcagctggtgtggatggcatgccattctgctgcatttgaagatttaagattgttaagcttcatcagagggacaaaagtatctcctcgggggaaactgtcaactagaggggtacaaattgcttcaaatgagaacatggataatatgggatcgagcactcttgaactgagaagcgggtactgggccataaggaccaggagtggaggaaacactaatcaacagagggcctccgcaggccaaaccagtgtgcaacctacgttttctgtacaaagaaacctcccatttgaaaagtcaaccatcatggcagcattcactggaaatacggagggaagaacttcagacatgagggcagaaatcataagaatgatggaaggtgcaaaaccagaagaagtgtcattccgggggaggggagttttcgagctctcagacgagaaggcagcgaacccgatcgtgccctcttttgatatgagtaatgaaggatcttatttcttcggagacaatgcagaagagtacgacaattaa

SEQ ID NO: 38 - DNA, 1497 bp, Unknown
  Deoptimized Influenza A virus
atggctagtcagggaacgaaaagatcttacgaacagatggagactgacggagataggcaaaacgctactgagatacgagctagcgtcgggaaaatgatcgacggaatcggaagattttacatacaaatgtgtacagagcttaaattgtccgatcacgaagggagattgatccaaaattcgttgacaatcgaaaaaatggtgcttagcgcattcgacgaaagacggaataagtatctcgaagaacaccctagtgccggtaaggatccaaaaaaaaccggagggcctatctataggagagtcgacggaaaatggatgagagagctcgtactatacgataaggaagagattagacggatatggcgacaagcgaataacggagaggacgcaaccgcaggattgacgcatattatgatatggcactctaatctaaacgacgcaacatatcaacggactagggcactcgttagaaccggtatggatcctagaatgtgctcacttatgcagggatctacattgcctagacggtcaggcgctgcaggcgctgcagtgaaagggatagggactatggttatggaactgataagaatggtgaaaagggggataaacgataggaatttttggagaggcgaaaacggacgaaaaactagatccgcatacgaaagaatgtgcaatatccttaaaggtaaatttcagactgcagcgcaacgcgctatggtcgatcaagtgagagagtctaggaatcccggtaatgccgaaatcgaagatctaatctttctcgctaggtccgcactcatacttaggggatccgttgcgcataaatcatgcttacccgcatgcgcatacggacccgcagtgtcaagcggatacgatttcgaaaaagaggggtatagtttagtcggaatcgatccattcaaattgctgcaaaatagtcagatatatagtctgattagacctaacgagaatcccgctcacaaatcgcaactcgtatggatggcatgccattccgcagcattcgaagatctgagattgttgtcattcattaggggaactaaagtgagtcctaggggtaagctatctactaggggggtgcaaatcgcatctaacgaaaatatggataatatggggtctagtacactcgaacttagatccgggtattgggcgatacggactagatccggggggaatactaatcagcaacgcgctagcgctggacagactagcgtgcaacctacatttagcgtgcaacggaatctgccattcgaaaaatctacaatcatggccgcattcacagggaataccgaaggacgaactagcgatatgagagccgaaatcattagaatgatggagggagcgaaacccgaagaggtaagttttagggggagaggggtattcgaactgtcagacgaaaaggcagcgaatccaatcgtaccgtctttcgatatgtctaacgaggggtcatactttttcggagataacgcagaggaatacgataattaa

SEQ ID NO: 39 - DNA, 1410 bp, Influenza A virus
atgaatccaaatcaaaagataataacgattggctctgtttctctcaccatttccacaatatgcttcttcatgcaaattgccatcttgataactactgtaacattgcatttcaagcaatatgaattcaactcccccccaaacaaccaagtgatgctgtgtgaaccaacaataatagaaagaaacataacagagatagtgtatctgaccaacaccaccatagagaaggaaatatgccccaaactagcagaatacagaaattggtcaaagccgcaatgtgacattacaggatttgcacctttttctaaggacaattcgattaggctttccgctggtggggacatctgggtgacaagagaaccttatgtgtcatgcgatcctgacaagtgttatcaatttgcccttggacagggaacaacactaaacaacgtgcattcaaacgacacagtacatgataggaccccttatcggaccctattgatgaatgagttaggtgttccatttcatctggggaccaagcaagtgtgcatagcatggtccagctcaagttgtcacgatggaaaagcatggctgcatgtttgtgtaacgggggatgataaaaatgcaactgctagcttcatttacaatgggaggcttgtagatagtgttgtttcatggtccaaagatatcctcaggacccaggagtcagaatgcgtttgtatcaatggaacttgtacagtagtaatgactgatgggagtgcttcaggaaaagctgatactaaaatactattcattgaggaggggaaaatcgttcatactagcacattgtcaggaagtgctcagcatgtcgaggagtgctcctgctatcctcgatatcctggtgtcagatgtgtctgcagagacaactggaaaggctccaataggcccattgtagatataaacataaagaattatagcattgtttccagttatgtgtgctcaggacttgttggagacacacccagaaaaaccgacagctccagcagtagccattgcttggatcctaacaatgaagaaggtggtcatggagtgaaaggctgggcctttgatgatggaaatgacgtgtggatgggaagaacgatcagcgagaagttacgcttaggatatgaaaccttcaaagtcattgaaggctggtccaaccctaattccaaattgcagataaataggcaagtcatagttgacagaggtaataggtccggttattctggtattttctctgttgaaggcaaaagctgcatcaatcggtgcttttatgtggagttgataaggggaagaaaagaggaaactgaagtcttgtggacctcaaacagtattgttgtattttgtggaacctcaggtacatatggaacaggctcatggcctgatggggcggacatcaatctcatgcctatataa

SEQ ID NO: 40 - DNA, 1410 bp, Unknown
  Deoptimized Influenza A virus
atgaatcctaaccaaaagattattacaatcggatccgttagccttactatatccacaatttgtttttttatgcaaatagcgatactgataactaccgttacattgcatttcaaacaatacgaattcaattcaccccctaataatcaggttatgttgtgcgaacctactattatcgaacggaatataaccgagatagtgtatctaacgaacactacaatcgaaaaagagatatgccctaagctcgcagagtatagaaattggtcaaaaccccaatgcgatataaccggattcgcaccatttagtaaggataatagtattaggttgtccgccggaggcgatatatgggttacacgcgaaccatacgtgtcatgcgatcccgataaatgctatcaattcgctctcggacagggaacgacattgaataacgtacattcaaacgataccgtacacgataggacaccttatagaacactattgatgaacgaactaggcgtacctttccatctcggaactaaacaggtttgtatcgcttggtctagtagctcatgccatgacggtaaggcatggttgcatgtgtgcgttaccggcgacgataaaaacgcaaccgctagtttcatatataacggtaggttagtcgatagcgtagtgagttggtctaaagacatactgcgaacacaggaatccgagtgcgtatgcataaacggtacatgtaccgtagtgatgaccgacggatccgctagcggtaaggccgatacgaaaatattgttcatagaggagggtaagatagtgcatacaagtacactatccggatccgctcaacatgtcgaagagtgctcatgttatcctagatatcccggcgttagatgcgtatgtagagacaattggaaagggtctaatagaccgatagtcgacattaatattaaaaactattcaatcgttagctcatatgtgtgttccggattagtcggcgatacccctagaaaaaccgatagctctagctcatcccattgtcttgaccctaataacgaagagggggggcatggcgttaagggatgggcattcgacgacggtaacgacgtttggatgggacggacaattagcgaaaaacttagattggggtatgagacttttaaggtaatcgaagggtggtctaatcctaattcgaaactgcaaattaataggcaagtgatagtcgatagggggaataggtccggatatagcggaatcttttccgttgagggtaagtcatgtattaataggtgtttttatgtcgaactgattagggggagaaaagaggaaaccgaagtgttatggactagtaactcaatcgttgtgttttgcggtacatccggtacttatggaaccggatcatggccagacggagccgatataaaccttatgccaatttaa

SEQ ID NO: 41 - DNA, 2274 bp, Influenza A virus
atggatgtcaatccgactttacttttcttgaaagtaccagtgcaaaatgctataagtaccacattcccttatactggagaccctccatacagccatggaacagggacagggtacaccatggacacagtcaacagaacacaccaatattcagaaaaagggaagtggacaacaaacacagagactggagcaccccaactcaacccaattgatggaccactacctgaggataatgagcccagtgggtatgcacaaacagattgtgtattggaagcaatggctttccttgaagaatcccacccagggatctttgaaaactcgtgtcttgaaacgatggaaattgtccaacaaacaagagtggataaattgacccaaggtcgccagacttatgactggacattgaataggaaccaaccggctgcaactgctttggccaacactatagaaatcttcagatcgaacggtctgacagcaaatgaatcaggacgactaatagatttcctcaaggatgtgatggaatcaatggataaggaagaaatggagataacaacacatttccagagaaagagaagagtaagggacaacatgaccaagaaaatggtaacacaaagaacaataggaaagaaaaaacaaaggctgaacaaaaggagctacctgataagagcactgacactgaacacaatgacaaaggatgcagaaagaggcaaattgaagaggcgagcaattgcaacacccggaatgcaaatcagaggattcgtgtactttgttgaaacactagcgaggagtatctgtgagaaacttgagcaatctggactcccagtcggagggaatgagaagaaagctaaattggcaaacgtcgtgaggaagatgatgactaactcacaggatactgaactctccttcacaattactggggacaatacaaaatggaatgagaatcagaatcctaggatgtttctggcaatgataacgtacatcacaaggaaccagccagaatggtttcgaaatgtcttaagtattgcccctataatgttctcaaacaagatggcgagattagggaaagggtacatgttcgaaagtaagagcatgaagttacgaacacaaataccagcagaaatgcttgcaaacattgatctcaaatacttcaatgaattaacgaaaaagaaaattgagaagataagacctctattaatagatggtacagcctcattgagccctggaatgatgatgggcatgttcaacatgctgagtacagtcctaggagtctcaatcctgaatcttggacagaaaaggtacaccaaaaccacatattggtgggacggactccaatcctctgatgattttgctctcatcgtaaatgcaccgaatcatgaggggatacaagcaggggtggataggttttataggacttgtaaactagttggaatcaatatgagcaagaagaagtcttacataaatcggacagggacatttgaattcacgagctttttctaccgctatggatttgtagccaatttcagtatggagctgcccagttttggagtgtctggaattaatgaatcggccgacatgagcattggtgttacagtgataaagaacaatatgattaacaacgaccttgggccagcaacagctcagatggctcttcagctattcatcaaggactacagatacacataccgatgccacagaggggatacgcaaatccaaacgaggagatcattcgagctgaagaagctatgggagcaaacccgttcaaaggcaggactgttggtttcagatgggggaccaaatctatacaacatccgaaatctccatattcctgaggtctgcttgaaatgggaattgatggatgaagactaccagggcagactttgcaatcctctgaatccattcgtcaaccataaggaaattgaatctgtcaacaatgctatagtaatgccagctcatggtccggccaagagtatggaatatgatgccgttgcaactacacattcatggattcctaaaaggaatcgttccattctcaatacgagtcaaaggggaattcttgaggatgaacagatgtaccaaaaatgctgcaatctattcgagaaattcttccccagcagttcatatcggaggccagttggaatttccagcatggtggaggccatggtgtctagggcccgaattgacgcacgaattgatttcgagtctggaaggattaagaaagaagagtttgctgagatcatgaagatctgttccaccattgaagagctcagacggcaaaaatag

SEQ ID NO: 42 - DNA, 2274 bp, Unknown
  Deoptimized Influenza A virus
atggacgtaaaccctacactgttgttccttaaggtgccagtgcaaaacgcaattagtacgacattcccttacacaggggatccaccatactctcacggaaccggtaccggatacactatggatacagtcaataggacacatcaatatagcgaaaagggtaagtggactactaacacagagacaggcgctccacaattgaaccctatcgacggaccgctaccagaggataacgaacctagcggatacgctcaaaccgattgcgtactcgaagctatggcattccttgaggaatcgcatccagggatattcgaaaatagttgtctcgagactatggagatagtgcaacagactagagtcgataaactgacacaggggagacagacatacgattggacacttaataggaatcaacctgccgcaaccgcactcgcaaatacaatcgaaatttttcgatctaacggactgacagctaacgaatccggaagattgatcgatttccttaaggacgttatggagtctatggataaggaggagatggagataacaacgcatttccaacggaaaagacgggttagggataatatgacaaaaaaaatggttacgcaacgaacaatcggtaagaaaaaacagagactgaataagagatcatatctgattagggcattgacactgaatacaatgactaaagacgccgaacgcggtaagcttaagagacgcgcaatcgcaacacccggaatgcaaattagggggttcgtatacttcgtcgagacactcgctaggtcaatttgcgaaaagctcgaacaatccggactgccagtcggagggaacgaaaaaaaagcgaaacttgcgaacgtcgttagaaagatgatgactaatagtcaggataccgaactatcttttacgattacaggcgataatacgaaatggaacgagaatcaaaaccctagaatgtttctcgcaatgataacatatattactaggaatcaacccgaatggtttaggaacgtactgtcaatcgcacctattatgtttagcaataagatggcaaggttgggtaaggggtatatgttcgaatcaaagtctatgaagcttagaacacagatacccgccgaaatgctcgctaacatagatcttaaatactttaacgagttaacgaaaaaaaagatcgaaaagattagaccactattaatcgacggaaccgctagcctatcccccggaatgatgatggggatgttcaatatgctatcgacagtgttaggggtgtcaatacttaacctagggcaaaaacggtatacaaagactacgtattggtgggacggactgcaatctagcgacgatttcgcattgatagtgaacgcccctaatcacgaagggatacaggccggagtcgatagattttacagaacatgtaagttagtcggaattaatatgtcaaaaaaaaagtcatacattaaccgaaccggaacattcgaattcactagctttttttacaggtacggattcgtcgctaactttagtatggagctaccgtcattcggcgtaagcggaattaacgaatccgccgatatgtcaatcggagtgacagtgattaagaataatatgattaataacgatctcggacctgcaaccgcacaaatggccctacaattgttcataaaggattatagatatacatataggtgtcataggggggatacacagattcagacacgacgatcattcgaactgaaaaagttgtgggagcaaactagatcgaaagccggattgctcgtaagcgacggagggccaaatctatacaatattaggaatctgcatatacccgaagtgtgtcttaagtgggagttgatggacgaggattaccaagggcgattatgcaatccgttgaatccattcgttaaccataaggaaatcgaatccgttaataacgcaatcgtaatgccagcacacggaccagctaagagtatggagtacgatgccgtcgcaacaacacatagttggataccgaaacgtaatagatcaatactgaatacaagccaaagggggatactcgaagacgaacaaatgtaccaaaaatgttgcaatctattcgaaaaatttttccctagtagttcatacaggcgaccagtcgggataagtagtatggtcgaggcaatggtgagtagggctaggattgacgctaggatcgatttcgaatccggacgaattaaaaaagaggaattcgcagagattatgaagatttgctctacaatcgaagagttacgtagacagaaatag

SEQ ID NO: 43 - DNA, 1704 bp, Influenza A virus
atggagaaaatagtgcttcttcttgcaatagtcagccttgttaaaagtgatcagatttgcatcggttaccatgcaaacaactcgacagagcaggttgacacaataatggaaaagaacgttactgttacacatgcccaagacatactggagaagacacataacgggaaactctgcgatctagatggagtgaagcctctgattctacgagattgtagtgtagctggatggctcctcggaaacccaatgtgtgacgaattcatcaatgtgccggaatggtcttacatagtggagaaggccaacccagccaatgacctctgttacccagggaatttcaacgactatgaagaactgaaacacctattgagcagaataaaccattttgagaaaattcagatcatccccaaaagttcttggtccgatcatgaagcctcatcaggggtgagctcagcatgtccataccagggaacgccctcctttttcagaaatgtggtatggcttatcaaaaagaacaatacatacccaacaataaagagaagctacaataataccaaccaggaaaatcttttgatactgtgggggattcatcattctaatgatgcagcagagcagataaagctctatcaaaacccaaccacctatatttccgttgggacatcaacactaaaccagagattggtaccaaaaatagccactagatccaaagtaaacgggcaaagtggaaggatggatttcttctggacaattttaaaaccgaatgatgcaatcaacttcgagagtaatggaaatttcattgctccagaatatgcatacaaaattgtcaaggaaggagactcagcaattatgaaaagtgaagtggaatatggtaactgcaacaccaagtgtcaaactccaataggggcgataaactctagtatgccattccacaacatacaccctctcaccatcggggaatgccccaaatatgtgaaatcaaacaaattagtccttgctactgggctcagaaatagtcctctaagagaaagaagaagaaaaagaggactatttggagctatagcagggtttatagagggaggatggcagggaatggtagatggttggtatgggtaccaccatagcaatgagcaggggagtgggtacgctgcagacaaagaatccactcaaaaggcaatagatggagtcaccaataaggtcaactcgatcattgacaaaatgaacactcagtttgaggccgttggaagggaatttaataacttggaaaggagaatagagaacttaaacaagaaaatggaagacggattcctagatgtctggacttataatgctgaacttctggttctcatggaaaatgagagaactctagacttccatgactcaaatgtcaagaacctttacgacagggtccgactacagcttagggataatgcaaaggagctgggtaacggttgtttcgagttctatcacaaatgtgataatgaatgtatggaaagtgtaagaaacggaacgtatgactacccgcagtattcagaagaagcaagattaaaaagagaggaaataagtggagtaaaattggaatcaatgggaacttaccaaatactgtcaatttattcaacagttgcgagttctctagcactggcaatcatggtggctggtctatctttgtggatgtgctccaatgggtcgttacaatgcagaatttgcatttaa

SEQ ID NO: 44 - DNA, 1704 bp, Unknown
  Deoptimized Influenza A virus
atggagaaaatagtgctactactcgcaatcgttagtctggttaagtccgatcagatatgcatagggtatcacgctaacaatagtaccgaacaggtcgacactattatggaaaaaaacgttaccgttacacacgcacaggacatactcgaaaaaacccataacggtaagttatgcgatttagacggagttaagccactgatacttagggattgttcagtcgccggatggttgttagggaatccaatgtgcgacgaattcattaacgtacccgaatggtcatacatagtcgaaaaagcgaatcccgctaacgatctatgttatccagggaattttaacgattacgaagagcttaagcatctactatctagaataaaccatttcgaaaagattcagataataccgaaatcgagttggtccgatcacgaagcgtcaagcggagtgagtagcgcatgcccataccaaggaacaccatcattctttagaaacgtcgtttggttgattaaaaaaaataatacatatccgactattaagagatcatataataatacaaaccaagagaatctactgatactatgggggatacaccatagtaacgacgcagccgaacagattaagctatatcagaatccaactacatacattagcgtagggactagtacacttaatcagagactcgtacctaaaatcgctactagatcgaaggtaaacggacaatccggtagaatggactttttttggactatactgaaacctaacgacgcaattaatttcgaatctaacggaaattttatcgctcccgaatacgcatataagatagtgaaagagggggatagcgcaattatgaaatccgaagtcgaatacggaaattgcaatactaagtgtcagacaccaatcggagcaattaactctagtatgccattccataacatacatccacttacaatcggagaatgccctaaatacgttaagtctaacaaactcgtactcgcaaccggacttaggaatagtccacttagagagagacgaagaaagagagggttgttcggagcaatcgcagggttcatagagggggggtggcagggtatggtcgacggatggtacgggtatcatcattctaacgaacagggatccggatacgcagccgataaagagagtactcagaaagcaatcgacggagtgacgaataaagtgaattcgataatcgataagatgaatacgcaattcgaagccgtaggtagggaattcaataatctcgagagacgaatcgaaaaccttaacaaaaaaatggaagacggattcctagacgtatggacttataacgccgaactgttagtgcttatggagaacgaaagaacccttgactttcacgattctaacgttaagaatctatacgatagagtgagactgcaattgagggataacgctaaagagttagggaacgggtgtttcgaattctatcataaatgcgataacgaatgtatggagtcagtgagaaacggtacatacgactatccgcaatattccgaagaggctagattgaaaagagaggagattagcggagtgaaacttgagtcaatggggacatatcagatattgtcaatatactcaaccgtcgctagtagtctcgcactcgcaattatggtcgccggactgtcactatggatgtgttcaaacggtagtctgcaatgtaggatttgtatataa

SEQ ID NO: 45 - DNA, 1497 bp, Influenza A virus
atggcgtctcaaggcaccaaacgatcttatgaacagatggaaactggtggagaacgccagaatgctactgagatcagggcatctgtcggaagaatggttagtggcattgggaggttctacatacagatgtgcacagagctcaaactcagtgactatgaagggaggctgatccagaacagcataacaatagagagaatggtactctctgcatttgatgaaagaaggaacagatacctggaagaacaccccagtgcggggaaagacccgaagaaaactggaggtccaatttaccggaggagagacggaaaatgggtgagggagctgattctatacgacaaagaggagatcaggaggatttggcgtcaagcaaacaatggagaggacgcaactgctggtcttactcacctgatgatatggcattccaatctaaatgatgccacatatcagagaacgagagctctcgtgcgtactggaatggaccccaggatgtgctctctgatgcaagggtcaactctcccaaggagatctggagccgccggtgcagcagtgaagggggtaggaacaatggtgatggagctgattcggatgataaaacgagggatcaacgaccggaacttctggagaggcgaaaatggaagaagaacaaggattgcatatgagagaatgtgcaacattctcaaagggaaattccaaacagcagcacaaagagcaatgatggatcaagtgcgagagagcagaaatcctgggaatgctgaaattgaggatctcatttttctggcacggtctgcactcatcctgagaggatcggtggcccataagtcctgcttgcctgcttgcgtgtatggacttgcagtggccagtggatatgactttgagagagaagggtactctctggttggaatagatcctttccgtctgcttcaaaacagccaggtctttagtctcattagaccaaatgagaatccagcacataagagtcaattagtgtggatggcttgccactctgcagcatttgaggaccttagagtctcaagtttcatcagaggaacaagagtggttccaagaggacagctatccaccagaggggttcaaattgcttcaaatgagaacatggaaacaatggactccaacacccttgaattgagaagtagatattgggcgataagaaccagaagcggaggaaacaccaatcagcagagggcttctgcaggacagatcagcgttcagcccactttctcggtacagagaaaccttcctttcgaaagagcgaccattatggcagcatttacaggaaatactgagggcagaacgtctgacatgaggactgaaatcataaaaatgatggaaagtgctagaccagaagatgtgtcattccagggacggggagtcttcgagctctcggacgaaaaggcaacgaacccgatcgtgccttcctttgacatgaataatgaaggatcttatttcttcggagacaatgcagaggagtatgacaattaa

SEQ ID NO: 46 - DNA, 1497 bp, Unknown
  Deoptimized Influenza A virus
atggctagtcaggggactaaacgatcatacgaacagatggaaaccggaggcgaacgacagaacgctacagagattagagcgagtgtgggacgtatggttagcggaatcggtagattctatatacagatgtgcacagagcttaagctatctgactatgagggaagactgatacagaattcgattacgatcgaaagaatggtgctatccgcattcgacgaaagaaggaataggtatctcgaagagcatcctagtgccggtaaggacccaaaaaaaaccgggggaccgatctatagacgtagagacggaaaatgggtgagagagcttatactgtatgacaaagaggagattagacggatttggagacaagcgaataacggagaggacgcaaccgcaggactgacacaccttatgatatggcactctaaccttaacgacgcaacttatcagagaactagagcactcgttagaaccggaatggaccctagaatgtgctcacttatgcagggatctacactccctagacggtctggcgcagccggagccgcagtgaagggagtcggaactatggttatggaactgattagaatgattaagagggggattaacgataggaatttttggagaggcgaaaacggaagacggactagaatcgcatacgaacggatgtgcaatatactgaaaggcaaattccaaaccgcagcgcaaagggcaatgatggaccaggtgagagagtctagaaatcccggtaacgcagagatcgaagacttaatctttctcgctagatccgctctcatactcagagggagtgtcgcacataaatcttgcctacccgcatgcgtatacggactcgcagtcgctagcggatacgatttcgaacgcgaagggtatagtctcgtcggaatcgacccattcagattgttgcagaattcgcaagtgtttagtctgattaggcctaacgagaatcccgctcacaaatcgcaactcgtttggatggcttgccattccgcagcattcgaagaccttagagtgagttcttttattaggggaactagggtagtgcctagggggcaactgtcaactaggggggtgcaaatcgcatctaacgagaatatggagactatggactctaatacactcgaactgagatctaggtattgggcaattagaactaggtccggagggaatacgaatcagcaacgagctagcgcaggacagattagcgttcagccaacatttagtgtgcaacggaatctgccattcgaaagagcgacaattatggccgcattcacagggaataccgagggtagaactagcgatatgcgtacagagataatcaaaatgatggagtccgctagaccagaggacgtaagttttcagggaaggggggtgttcgaactgtctgacgaaaaggcaacgaatccgatagtgccatcattcgatatgaataacgagggatcttattttttcggagataacgccgaagagtacgataactaa

SEQ ID NO: 47 - DNA, 1350 bp, Influenza A virus
atgaatccaaatcagaagataataaccattgggtcaatctgtatggtaattggaatagttagcttaatgttacaaattgggaacataatctcaatatgggtcagtcattcaattcaaacagggaatcaacaccaagatgaaccaatcagaaatgctaattttcttactgagaacgctgtggcttcagtaacattagcgggcaattcatctctttgccccgttagaggatgggctgtacacagtaaagacaacagtataaggattggttccaagggggatgtgtttgttattagagagccgttcatctcatgctcccacttggaatgcagaactttctttttgactcagggagccttactgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacaattggaatttctggcccagacaatggggctgtggctgtattgaaatacaatggcataataacagacaccatcaagagttggaggaacaacatactgagaactcaagagtctgaatgtgcatgtgtaaatggctcttgctttactgtaatgactgatggaccaagtaatgggcaggcatcatataagatcttcaaaatggaaaaaggaaaagtggttaaatcagtcgaattgaatgcccctaattatcactatgaggaatgctcctgttatcctgatgctggcgaaatcacatgtgtgtgcagggataattggcatggctcaaataggccatgggtatctttcaatcagaatttggagtatcaaataggatatatatgtagtggagttttcggagacaatccacgccccaatgatggaacaggtagttgtgatccagtgtcccctaacggggcatatgggataaaagggttttcatttaaatacggcaatggtgtttggatcggaagaaccaaaagcactaattccaggagtggttttgaaatgatttgggatccaaatgggtggactgaaacggacagtagcttttcagtgaaacaagatatagtagcaataactgattggtcagggtatagcgggagttttgttcagcatccagaactgacaggattagattgcataagaccttgcttctgggttgagttaatcagagggcggcccaaagagagcacaatttggactagtgggagcagcatatctttttgtggtgtaaatagcgacactgtgagttggtcttggccagacggtgctgagttgccattcaccattgacaagtag

SEQ ID NO: 48 - DNA, 1350 bp, Unknown
  Deoptimized Influenza A virus
atgaatccgaatcaaaaaattataacaatagggtcaatctgtatggtaatcggtatagtgtcacttatgttacaaatcgggaatattatatctatttgggtgtcacactcaatccaaaccggtaatcaacaccaagacgaacctatacggaatgcgaatttcttaacagagaatgccgtagctagcgttacgttagccggtaatagttcattgtgtcccgttagggggtgggctgtgcatagtaaggataatagtattaggatagggtctaaaggcgacgtattcgtgatacgcgaaccttttatctcttgctcacacttagagtgtagaacattttttctgactcaaggcgcactgttaaacgataaacactctaacggtacagttaaggataggtcaccacataggacattgatgtcatgtcccgtaggcgaagctcctagtccatataatagtagattcgaaagcgttgcatggtccgctagcgcttgtcacgacggaactagttggttgacaatcgggatatccggacccgataatggcgcagtcgcagtgttgaagtataatgggattataaccgatactatcaaatcatggagaaataatatactgagaacacaggagtccgaatgcgcttgcgttaacggatcatgctttaccgttatgactgacggaccatctaacgggcaagctagttataaaattttcaaaatggagaaaggtaaggtagtgaaatccgttgagcttaacgctccaaattatcattacgaagagtgtagttgctatccagacgctggcgaaattacttgcgtatgtagagacaattggcacggatctaatagaccatgggttagctttaatcagaatttagagtatcagatagggtatatatgttccggagtgttcggcgataatcctagacctaacgacggtacagggtcatgcgatccagtgagtccaaacggcgcatacggaattaaagggtttagctttaagtatgggaatggcgtatggatcggtaggactaagtctactaatagtagatccggattcgaaatgatatgggaccctaatgggtggactgagactgatagtagttttagcgtaaaacaggatatagtcgctataaccgattggagcgggtatagcggatcattcgtacagcatcccgaattgactgggttagactgtattagaccatgcttttgggtcgaattgattagggggagaccaaaagagtcaactatatggactagcggatctagtattagtttttgcggagtgaattccgataccgttagttggtcatggccagacggagctgagttgccatttacaatcgataaatag

SEQ ID NO: 49 - DNA, 2274 bp, Influenza A virus
atggatgtcaatccgactttacttttcttgaaagttccagcgcaaaatgccataagcaccacattcccatatactggagatcctccatacagccatggaacgggaacaggatacaccatggacacagtcaacagaacacatcaatattcagaaaaggggaaatggacaacaaacacagaaactggagcaccacaactcaacccaattgatggaccattacctgaggataatgagccaagtggatatgcacaaacagattgtgtcctggaagcaatggctttccttgaagagtcccacccaggaatctttgaaaactcgtgtctcgaaacgatggaagttgttcagcaaacaagagtggacaagctgactcaaggtcgccagacctatgattggacattgaacaggaatcagccggctgcaactgcattagctaatactatagaggttttcagatcaaacggtctaacggccaatgaatcaggaaggctgatagacttcctcaaggatgtgatggaatcaatggacaaagaagaaatggagataacaacgcacttccaaagaaaaagaagagtaagggacaacatgaccaagaaaatggtcacacaaagaacaataggaaagaagaagcagagattaaacaagagaagttatttaataagagcattgacactgaacacaatgacaaaagacgctgaaagaggcaagttaaagagaagagcaattgcaacacccgggatgcaaatcagaggatttgtgtattttgttgaaacattggcgagaagcatctgtgagaagcttgaacagtctgggctcccagtcggaggcaatgaaaagaaggctaaactggcaaatgtcgtgaggaaaatgatgactaactcacaggacacagagctctcttttacaatcactggagacaacaccaaatggaatgaaaatcagaaccctagaatgtttctggcaatgataacatacataacaagaaatcaacctgaatggttcaggaatgtcttgagcatcgcacctataatgttctcgaataaaatggcaaggctagggaaaggatacatgtttgaaagcaaaagcatgaagcttcgaacacaggtatcagcagaaatgctagcaaatattgacctgaagtatttcaatgaatcaacaaaaaagaaaatagagaagataaggcctcttttaatagatggcacagcctcattgagtcccggaatgatgatgggcatgttcaacatgctaagcacagttttaggagtttcaatcctaaatctgggacaaaagaaatacaccaaaacaacgtattggtgggacggactccaatcctctgatgactttgctctcatagtgaatgcactgaatcatgagggaatacaagcaggagtagatagattctataggacttgcaaactagtcggaatcaatatgagcaaaaagaagtcctacataaacaggacaggaacgtttgaattcacaagctttttctatcgctatgggttcgtagccaatttcagcatggaactgcccagctttggagtgtctgggatcaatgaatcggctgacatgagcattggggtaacagtgataaagaacaacatgataaacaatgaccttgggccagcaacggcccaaatggctctccagctgttcatcaaggattacagatatacataccggtgccacagaggggacacacaaatccagacaaggagatcattcgagctgaagaaattatgggaacaaacccgatcaaaggcgggactgctggtttccgatgggggaccaaacctgtacaatatccgaaatctccacattccggaagtctgcttgaaatgggagctgatggacgaagaatatcagggaaggctttgtaaccccttgaacccatttgtcagccataaggagatagagtctgtgaacaatgcagtggtgatgccagctcacggcccagccaaaagcatggaatatgatgctgttgctactacgcattcctggatccccaagaggaatcgctccattcttaacacgagtcaaaggggaatcctcgaagatgaacagatgtatcaaaagtgctgcaatctattcgaaaagttcttccctagcagttcgtacagaagaccggtcgggatttctagcatgggggaggccatggtatctagggcccgaattgatgcgcgaattgacttcgaatctggacggattaagaaagaggagtttgctgagatcatgaagatctgttccaccattgaagaactcagacggcagaaatag

SEQ ID NO: 50 - DNA, 2274 bp, Unknown
  Deoptimized Influenza A virus
atggacgttaatccgacactgttattccttaaagtgccagcgcaaaacgcaatctctacgacattcccatatacaggcgatccaccttatagtcacggaaccggaacagggtatacaatggatacagtgaatagaacacaccaatactccgaaaagggtaagtggacaacgaataccgagacaggggcaccacaattgaacccaatcgacggaccattgcccgaagataacgaacctagcggatacgcacaaaccgattgcgtactcgaggctatggcctttctcgaagagtctcaccctgggatattcgaaaactcatgtctcgaaactatggaggtcgtgcaacaaacaagagtcgacaaactgactcaggggagacagacatacgattggacactgaataggaatcagccagccgcaaccgcactagcgaatacaatcgaagtgtttagatcaaacggattgaccgctaacgaatccggaaggttgatcgatttccttaaggacgttatggagtcaatggataaggaggaaatggagataacaacgcattttcagagaaagagaagggtgagagacaatatgacaaaaaagatggttacgcaacggacaatcggaaagaaaaaacagagactgaataagcgatcatatctgatacgcgcactaacgttgaacactatgactaaggacgccgaaaggggaaagcttaaaagacgcgcaatcgcaacacccggtatgcagatacgcggattcgtatatttcgtcgagacactcgctagatccatttgcgaaaagctcgaacaatccggactgccagtcggggggaacgaaaaaaaagcgaaactcgctaacgtcgttagaaagatgatgactaattcgcaagacacagagcttagctttacgataacaggcgataatacgaaatggaacgagaatcagaaccctagaatgtttctcgctatgataacatacataacacgaaaccaacccgaatggtttagaaacgtactatcaatcgcacctataatgtttagcaataagatggctaggttagggaaagggtatatgttcgaatctaagtctatgaaactgagaacgcaagttagcgccgaaatgctcgcaaatatcgatcttaagtactttaacgaatcaacaaaaaaaaaaatcgaaaagattaggccactactgatagacggaaccgctagcttaagccctggaatgatgatgggaatgttcaatatgttgagtacagtgttaggcgtaagcatactgaatctgggacagaaaaagtatacaaagactacttattggtgggacggactgcaatctagcgacgatttcgcattaatcgttaacgcactgaatcacgaagggatacaggccggagtcgataggttctatagaacatgtaagttagtcggaattaacatgagtaaaaaaaagtcatacattaatagaaccggaacattcgaattcacaagctttttttataggtacggattcgtcgctaacttttcgatggagttaccgtcattcggagtgagcggaattaacgaatccgccgatatgtcaatcggagttacagtgataaagaataatatgattaataacgatctcggaccagcaaccgcacaaatggccttacaactgttcattaaggactataggtatacatatagatgtcataggggcgatacgcaaatacagactagacggtcattcgaactgaaaaagttatgggaacagactagatcgaaagccggattgctcgtaagcgacgggggaccgaatctatacaatattagaaatctgcatatacccgaagtgtgtcttaagtgggagttgatggacgaagagtatcagggacgactatgcaacccactgaatccattcgttagccataaggagatcgaatccgttaataacgcagtcgtaatgccagctcacggacccgctaagtctatggaatacgacgcagtcgcaactacacatagttggataccgaaaagaaataggtcaatacttaacacaagccaaagggggatactcgaagacgaacagatgtaccaaaaatgttgcaatctattcgaaaagtttttcccaagctctagctatagacgacctgtcgggattagctctatgggcgaagcgatggtgagtagggctagaatcgacgctaggatcgatttcgaatccggacgaataaaaaaggaggaattcgcagagattatgaagatttgctcgacaatcgaagagcttagacggcaaaagtag

SEQ ID NO: 51 - DNA, 1659 bp, Influenza A virus
atgaacattcaaattctggcattcattgcttgtgtgctgactggagctaaaggagacaaaatatgtcttgggcaccatgctgtggcaaatggaacaaaagtgaacacattaacagagagggggattgaagtagtgaatgccacagagacagttgaaactgcgaatatcaagaaaatatgtactcaagggaaaagaccaacagatctgggacaatgtggacttctagggaccctaataggacctccccaatgtgatcaattcctggagttttcctctgatttgataattgagcgaagagaaggaaccgatgtatgctatcccggtaaattcacaaatgaagaatcactgagacagatccttcgaagatcaggaggaattggtaaggagtcaatgggcttcacctatagtggaataaggaccaatggagcgacaagtgcctgcacaagatcaggttcttctttctatgcagagatgaagtggttgctgtcgaattcagacaatgcagcattcccacagatgacaaaatcgtatagaaatcccagaaacaaaccagctctgataatttggggagttcatcactctgaatcggttagcgagcagaccaaactctatggaagtggaaacaagttgataaaagtaagaagctcaaaataccaacaatcatttaccccaaatcctggagcacggagaatcgatttccactggctactcctggatcccaatgacacagtgaccttcactttcaatggggcattcatagcccctgacagggcaagtttctttagaggagaatcaataggagtccagagtgatgctcctttggattctagttgtggagggaattgctttcacagtgggggtacgatagtcagttccctgccattccaaaacatcaaccctagaactgtgggaaaatgccctcggtatgtcaaacagaaaagcctccttctggctacaggaatgagaaatgttccagagaaaccaaagaaaagaggcctttttggagcaattgctggattcatagagaacggatgggagggtctcatcaatggatggtatggtttcagacatcaaaatgcacaaggagagggaactgcagctgactacaaaagcacccagtctgcaatagatcagatcacaggcaaattgaatcgtctaattggcaaaacaaatcagcagtttgggctgatagacaatgagttcaatgaggtagaacaacaaataggaaatgtcattaattggacacaagacgcaatgactgagatatggtcgtataatgctgagctgttggtggcaatggaaaatcaacatacaatagatcttacggattcagaaatgagcaaactttatgagcgtgtcagaaaacaactgagggagaatgctgaagaagatgggactggatgtttcgaaatattccataagtgtgatgatcattgtatggagagcataagaaacaacacttatgaccatactcaatacagaacagagtcactgcagaatagaatacagatagacccagtgaaattgagtagtggatacaaagacataatcttatggtttagcttcggggcatcatgttttcttcttctagccattgcaatgggattggttttcatttgcataaaaaatggaaacatgcagtgcactatttgtatatag

SEQ ID NO: 52 - DNA, 1659 bp, Unknown
  Deoptimized Influenza A virus
atgaatatacagatactcgcattcatagcttgcgtacttaccggagctaaaggcgataagatatgtctagggcatcacgcagtcgcaaacggaacgaaagtgaatacacttacagagagagggatagaggtcgttaacgctacagagacagtcgaaaccgcaaatattaaaaaaatttgtacacaaggaaaacgaccaaccgatctgggacaatgcggactgttagggacactgataggaccaccacaatgcgatcaattccttgagtttagtagcgatctgataatcgaacgaagagagggaactgacgtttgttatcccggtaagttcactaacgaagagagtcttagacagatactgagacggtcagggggaatcggaaaagagtcaatggggtttacgtattctgggattaggactaatggcgcaactagcgcatgtactagaagcggatcatcattctatgccgaaatgaaatggttgttgtcgaattccgataacgctgcattcccacaaatgactaaatcgtatagaaatcctaggaataaacccgcactgataatatggggagtgcatcatagcgaatccgtaagtgaacagactaaattgtacggatcaggtaataaactgattaaagtgagatctagtaagtatcagcaatcgtttacacctaatcccggagctagacgtatcgatttccattggctattgctcgaccctaacgataccgttacattcacattcaatggcgcattcatagcgccagatagggcaagtttttttagaggcgaatcaatcggagtgcaatcagacgcaccacttgactcaagttgcggagggaattgtttccatagcggagggactatagtgagtagtctgccattccaaaatattaatcctagaacagtgggtaagtgtcctagatacgttaaacagaaaagtctgttactcgcaaccggaatgcgtaacgtacccgaaaaacctaaaaaaaggggattgttcggagcgatagccggattcatagagaatggatgggagggactgattaacggatggtacggatttagacaccaaaacgctcagggagagggaaccgcagccgattataaatcgacacaatctgcaatcgatcagattaccggtaagcttaatagattgattggtaagactaatcagcaattcggactgatagacaatgagtttaacgaagtcgagcaacagatagggaatgtgattaattggacacaagacgctatgactgagatttggtcttataatgccgaactgctagtcgctatggagaatcaacacacaatcgatctaaccgatagcgaaatgtcaaaattgtatgagagagtgagaaaacagcttagagagaatgcagaggaagacggaactgggtgtttcgagatattccataaatgcgacgatcactgtatggaatctattagaaataatacatacgatcatacacagtatagaacagagtcacttcaaaatcggatacagatagacccagttaaactatctagcggatataaagacataatactgtggttctcattcggagctagttgttttctgttgctcgcaatcgctatgggacttgtattcatatgtattaaaaacggtaatatgcaatgtacaatttgcatatag

SEQ ID NO: 53 - DNA, 1497 bp, Influenza A virus
atggcgtctcaaggcaccaaacgatcttatgagcagatggaaactggaggggaacgccagaatgccactgagatcagagcatctgttgggagaatggttggtggaattgggagattctacatacagatgtgtactgaactcaaactcagtgactatgaaggaagactgatccaaaacagcataacaatagagagaatggttctctcggcatttgatgagagaagaaatagatatctggaagagcatcccagtgctggaaaagaccctaagaaaactggaggcccaatctacaggaggagagatgggaaatgggtgagagaattgatcctgtatgacaaggaggagatcaggaggatttggcgtcaagcaaataatggagaagatgcgactgctggtctcacccatttgatgatctggcattccaatctgaatgatgccacatatcagaggacaagggcacttgtgcgcagtgggatggaccccagaatgtgctctctgatgcaaggctcaactcttccgaggagatctggagcagccggagcagcagtaaaaggagttggaacaatggtgatggaattggtccggatgatcaagcggggaatcaatgataggaatttctggagaggcgaaaatggacggaaaacaagaattgcttacgaaagaatgtgcaacattctcaaggggaaattccaaacagcagcacaacgagcaatgatggaccaggtaagggaaagccggaatcctgggaatgctgaaattgaggatctcatcttcctggcacgatctgctctcattctgagaggatcagtggctcacaaatcctgtctgcctgcttgtgtgtatggacttgctgtagccagtggatacgattttgaaagagaaggatactccctagttggaattgatcctttccgcctgctccaaaacagtcaagtcttcagccttatcaggccgaacgaaaatccagcacataaaagtcaactggtatggatggcatgccactctgcagcatttgaagacctaagagtgtcaagcttcatcagaggaacaaaagtggttccaagagggcaactgtccaccagaggagtccaagtcgcttcaaatgagaacatggagacgatggattccagcactcttgaattgagaagtagatactgggctataagaaccagaagtggaggaaacacaaatcagcagagagcgtccgcagggcaaatcagcgtacagccaacattctctgtccagagaaaccttccattcgagagagcaaccattatggcggcatttacagggaacactgaaggcagaacttcagacatgagaactgagataataaggatgatggaaaatgccaaacctgaagatgtgtctttccaagggcggggagtcttcgagctatcggacgaaaaggcaacgaacccgatcgtgccttcctttgacatgagtaacgaagggtcttatttcttcggagacaatgcagaggagtatgacaattga

SEQ ID NO: 54 - DNA, 1497 bp, Unknown
  Deoptimized Influenza A virus
atggctagtcagggaacaaaaagatcatacgaacagatggagacaggcggagagagacaaaacgctaccgaaattagggcaagcgtagggagaatggtcggcggaatcggaaggttctatatccaaatgtgtacagagcttaaattgtccgattacgagggtagactgatacagaattcgattacaatcgaaagaatggtgcttagcgcattcgacgaaagacgtaatcggtatctcgaagagcaccctagcgcaggtaaggatccaaaaaaaaccggaggaccaatctatagacggagagacggaaaatgggtgagagagttgatactgtatgacaaagaggaaatcagaagaatctggcgacaagcgaataacggcgaagacgctactgccggactgacacaccttatgatatggcatagtaatctgaacgacgcaacatatcaacggactagggcactcgttagatccggaatggaccctagaatgtgctctcttatgcaggggagtacactccctagacgatccggagccgcaggcgcagccgttaagggagtgggaactatggttatggaactcgttagaatgatcaaaagggggattaacgataggaatttttggagaggcgaaaacggaagaaagactagaatcgcatacgaacggatgtgtaatatactgaaagggaaattccaaaccgcagcgcaacgcgctatgatggatcaggttagggagtctagaaatcccggaaacgcagaaatcgaagacctaatctttctcgctagatccgcactgatacttagggggtctgtcgcacataaaagttgtctaccagcatgcgtatacggactcgcagtcgctagcggatacgatttcgaacgcgaagggtatagtctagtcggaatcgatccgtttagattgttgcagaattcgcaagtgttctcactgattagacctaatgagaatcccgcacataagtctcaactcgtatggatggcatgccattccgcagcattcgaagaccttagagtgagttcattcataagggggactaaggtcgtgcctagggggcaactgtctactaggggagtgcaagtcgctagtaacgagaatatggagacaatggactctagtactctcgaactgagatctagatattgggcgattagaactagatccggagggaatacgaatcagcaacgcgcatccgccggacagattagcgtgcaacctacattctcagtgcaacgaaatctgccattcgaaagggctacgattatggccgcattcacagggaataccgagggacggactagcgatatgagaaccgaaattatcagaatgatggagaacgctaaaccggaagacgtaagttttcaggggagaggggtattcgaactgtctgacgaaaaagcgactaatccaatcgttccgtcattcgatatgtctaacgagggatcttattttttcggagataacgctgaggaatacgataattga

SEQ ID NO: 55 - DNA, 1362 bp, Influenza A virus
atgaatccaaatcagaagataataacaattggctccgtctctctaaccattgcaacagtatgtttcctcatgcagattgccattctagcaatgactgtaacactgcatttcaggcaaaatgaatgcagcatttccgcgaacagtcaggtagtgccgtgtgaaccaactacagagaaagaggtctgttcgaacgtagtagactatagaagctggtcaaagccgcagtgtcaaattacaggatttgcccctttttccaaggacaactcaattcgactttctgctggtggagacatttggataacaagagagccttatgtgtcgtgtgacaccagcaaatgttaccaatttgcacttgggcaggggaccacactggataacaaacattcaaacggaacaatacatgatagaatctcccatcggacccttttgatgaatgaactgggtgttccatttcacttgggaaccaaacaagtttgcatagcatggtccagctcaagttgccatgatgggaaagcatggttgcacgtttgtgtcactggggatgatagaaatgcaactgctagtttcatttacaatgggatgcttgttgacagtattggttcatggtctcaaaatatcctcaggacccaggagtcagaatgcgtttgcatcaatgggtcttgtacagtagtgatgactgatggaagtgcctcagggaaggccgatactaggatattattcgtcaaagaaggaaagattgttcacattagcccattgtcaggaagtgctcagcatatagaggaatgttcctgttatccccgatacccaaacgtcagatgtgtctgcagggacaactggaagggctctaataggcctgttatagacataaacatggcagattatagcatcgactccagttatgtgtgctcaggactcgttggggacacaccaaggaatgaggatagttctagcagcagcaactgtagggatcccaatgaagagaggggaaacccaggagtgaaaggatgggcctttgacagtggagatgatgtttggatgggtagaacaatcagtagggattcgcggtcaggctatgagacatttagggtcattggtggttggaccactgccaattccaaatcacagaccagcagacaagtcatagttgataataacaattggtctggttattctggtattttctctgttgaacacaaaagctgtatcaataggtgtttttatgtggagttaataagaggaaggccgaaagaaactagagtatggtggacctcaaacagtattgtcgtgttttgtggcacttctggcacttatggaacaggctcatggcctgatggggcgaacatcaatttcatgcctatataa

SEQ ID NO: 56 - DNA, 1362 bp, Unknown
  Deoptimized Influenza A virus
atgaatccgaatcagaaaatcattactatcggatccgttagcttgacaatcgcaaccgtatgttttcttatgcagattgcgatactcgcaatgaccgttacattgcattttagacaaaacgagtgttctattagcgctaactctcaggtcgtgccatgcgaacctacaaccgaaaaagaggtttgttcaaacgtagtcgattataggtcatggtctaaaccgcaatgtcagattaccggattcgcaccattttcgaaagacaattcgattagactatccgccggaggcgatatttggataactagggaaccatacgtgtcatgcgatacaagtaagtgttatcaattcgcactcggccaagggactacactcgataacaaacactctaacggtacaatacacgataggattagtcataggacactgcttatgaacgagttaggcgtaccattccatctgggaactaaacaggtatgcatagcctggtcatctagttcatgtcacgacggtaaggcatggttgcacgtatgcgtaaccggcgacgatagaaacgctaccgcctcattcatatataacggtatgctagtcgactcaatcgggtcatggtcacaaaatatacttaggacacaggaatccgaatgcgtatgtattaacggatcatgtacagtcgttatgaccgacggatccgctagcggtaaggccgatacacggatactgttcgttaaagagggtaagatagtgcatattagcccacttagcggatccgcccaacatatcgaagagtgttcatgttatcctagatatccgaacgttaggtgcgtttgtagggataattggaaagggtctaatcgacccgttatcgatattaatatggccgattatagtatcgatagttcatacgtttgttccggattagtcggcgatactcctagaaacgaagatagttctagctctagtaattgtagagacccaaacgaagagagagggaatcccggagtgaaagggtgggcattcgatagcggtgacgacgtttggatgggtaggacaattagtagggactctagatccgggtatgagacttttagggtgataggcggatggacaaccgcaaactctaagagtcagactagtagacaggtgatagtcgataataataattggtccgggtatagcgggatttttagcgtcgagcataagtcatgtattaatcggtgtttttatgtcgaattgattagggggcgacctaaagagactagggtgtggtggactagcaattcgatagtcgttttttgcggtactagcggaacatacggaaccggaagttggccagacggagcgaatattaattttatgcctatataa

SEQ ID NO: 57 - DNA, 2274 bp, Influenza A virus
atggatgtcaatccgactttacttttcttgaaagttccagcgcaaaatgccataagcaccacattcccatacactggagatcctccatacagccatggaacgggaacaggatacaccatggacacagtcaacagaacacaccaatattcagaaaaggggaaatggacaaccaacacagagactggagcaccccaacttaacccaattgacggaccactgcctgaggacaatgagccaagtggatatgcacaaacagactgtgtccttgaagcaatggctttccttgaagagtcccacccaggaatctttgaaaactcgtgtcttgaaacgatggaagttgttcaacaaacaagagtggacaaactaactcaaggtcgtcagacctatgattggacattaaacaggaatcaaccggctgcaactgcattagccaatactatagaggtcttcagattgaacggtctgacagctaatgaatcaggaaggctaatagatttcctcaaagatgttatggagtcaatggataaagaggaaatggaaataacaacacacttccaaagaaaaagaagagtgagggacaacatgaccaagaaaatggtcacacaaagaacaataggaaagaagaaacaaaggctaaacaagagaagctatctaataagagcactgacactgaacacaatgacaaaagacgctgaaagaggcaaactgaagagaagagcaattgcaacacccggaatgcaaatcagaggatttgtatactttgttgaaacattggcaaggagcatttgtgagaagcttgaacaatctgggctcccggttggaggtaatgaaaagaaggctaaactggcaaatgttgtgagaaaaatgatgactaattcacaagacacagagctctctttcacaatcactggagacaacaccaaatggaacgaaaatcaaaaccccagaatgtttctggcaatgataacatacataacaagaaaccaacctgaatggtttaggaatgtcttgagcattgcacctgtaatgttctcaaataaaatggcaagactagggaaaggatacatgttcgaaagcaagagcatgaagcttcgaacacaaataccggcagaaatgctagcaaatattgatctgaaatatttcaatgagtcaacaaagaagaaaatagagaagataaggcctcttctgatagatggtacagcctcattgagccctggaatgatgatgggcatgttcaacatgctaagtacagtcttgggagtctcgattctaaatctagggcaaaagaggtacaccaaaacaacatactggtgggacggactccaatcctccgatgactttgctctcatagtgaatgctccgaatcatgagggaatacaagcaggagtagatagattctataggacctgcaagctggtcggaatcaacatgagcaaaaagaagtcctacataaacaggacaggaacatttgaattcacaagctttttctatcgctacggatttgtagccaattttagcatggaactgcccagttttggagtatctggaattaatgaatctgccgacatgagcattggagtaacagtgataaagaacaacatgataaacaatgaccttggaccagcaacagctcaaatggctcttcagctgttcatcaaggattacagatacacgtaccggtgccacaggggggacacacaaattcagacaaggaggtcattcgaactgaaaaagttgtgggaacaaacccgctcaaaggcaggactgttggtttcagatggagggccaaacttatacaatattcggaatctccacattccggaagtctgcctgaagtgggggctgatggacgaagactatcagggaaggctctgtaatcctctgaatccatttgtcagccacaaagagatagagtctgtaaacaatgctgtggtgatgccagctcatggtcctgccaagagcatggaatatgatgctgttgctaccacacactcctggatccctaagaggaaccgctccatcctcaacacaagccaaaggggaatccttgaagatgaacagatgtatcaaaagtgctgcaatctattcgagaaattcttccctagcagttcatacaggagaccggttggaatttccagcatggtggaggccatggtttccagggcccgaattgatgcgcgaattgacttcgaatctggacggattaagaaggaggagtttgctgagatcatgaagatctgttccaccattgaagagctcagacggcagaaatag

SEQ ID NO: 58 - DNA, 2274 bp, Unknown
  Deoptimized Influenza A virus
atggacgttaatccgacattgctattccttaaggtacccgcacagaacgctattagcacaacattcccatatacaggcgatccgccatactcacacggaaccggaaccggatacacaatggacacagttaacagaacacaccaatactccgaaaagggtaagtggacaactaataccgaaaccggagcaccacaactgaatccgatagacggaccactgccagaggataacgaacctagcggatacgcacaaaccgattgcgtactcgaagctatggcatttctcgaagagtcacaccctgggatattcgaaaactcatgtctcgagacaatggaggtagtgcaacagacaagggtcgataagttgacacagggacgacagacatacgattggacactgaataggaatcaacctgccgcaaccgcattggcgaatacaatcgaagtgtttagacttaacggattgaccgctaacgaatccggtaggttgatcgattttctgaaagacgtaatggagtctatggacaaagaggagatggagattacgacacactttcagagaaagagacgcgttagggataatatgactaaaaagatggttacgcaacggacaatcggaaaaaaaaagcaacgactgaataagagatcctatctgatacgcgcactgacactgaatactatgacaaaagacgccgaaaggggtaagcttaagagacgcgcaatcgctacacccggaatgcaaattagggggttcgtatacttcgtcgagacactcgctagatcgatttgcgaaaagctcgaacaatccggactgccagtcggaggaaacgagaaaaaagcgaaactcgctaacgtcgttaggaaaatgatgacaaatagccaagacacagagcttagctttacaattaccggagacaatacaaaatggaacgagaatcaaaaccctagaatgttccttgcaatgataacatacataactaggaaccaacctgaatggtttagaaacgtactgtcaatcgcacccgttatgtttagcaataaaatggctagattgggtaagggatatatgttcgaaagtaagtctatgaaacttagaacacaaattccagccgaaatgctcgctaacatagaccttaagtactttaacgaatcgacaaaaaaaaaaatcgaaaagattagaccattactgatagacggaaccgctagcctatccccaggaatgatgatgggaatgttcaatatgctatcaaccgtactcggagtgagcatactgaacctagggcaaaagagatacactaagacaacatattggtgggacggactgcaatcaagcgacgatttcgcactgatagtgaacgcacctaatcacgaagggatacaagccggagtcgataggttttatagaacatgtaagttagtcggaataaatatgtcaaaaaaaaagtcatacattaataggaccggaacattcgaattcacaagctttttttataggtacggattcgtcgctaactttagcatggagctacctagtttcggcgttagcggaattaacgaatccgccgatatgtcaatcggagtgacagtgattaagaataatatgattaataacgatctagggcctgcaacagcccaaatggcactgcaactattcataaaggattacagatatacatataggtgtcataggggggatacgcaaatacagactagacggtcattcgagcttaagaagttgtgggagcaaactagatctaaggccggactgttagttagcgacggaggacctaacctatataatattaggaacctacacataccggaagtgtgtcttaagtggggacttatggacgaagattaccaaggtaggttgtgcaatccgttaaacccattcgttagccataaagagatagagtccgttaataacgccgtcgttatgcccgcacacggaccggctaagtctatggaatacgacgcagtcgcaacaacacatagttggataccgaaacggaataggtccatactgaatactagccaaagggggatactcgaagacgaacaaatgtaccaaaagtgttgcaatctattcgaaaaatttttccctagctctagctatagacgaccagtcggaattagctcaatggtcgaagctatggtgagtagggctagaatcgacgctagaatcgatttcgaatccggacgtattaagaaagaggaattcgcagagattatgaaaatttgctctacaatcgaagagcttagacggcaaaagtag

SEQ ID NO: 59 - DNA, 1704 bp, Influenza A virus
atgaatactcaaattttggcattcattgcttgtatgctgattggaactaaaggagacaaaatatgtcttgggcaccatgctgtggcaaatgggacaaaagtgaacacactaacagagaggggaattgaagtagtcaatgccacggagacggtggaaactgtaaatattaagaaaatatgcactcaaggaaaaaggccaacagatctgggacaatgtggacttctaggaaccctaataggacctccccaatgcgatcaatttctggagtttgacgctaatttgataattgaacgaagagaaggaaccgatgtgtgctatcccgggaagttcacaaatgaagaatcactgaggcagatccttcgagggtcaggaggaattgataaagagtcaatgggtttcacctatagtggaataagaaccaatggggcgacgagtgcctgcagaagatcaggttcttctttctatgcggagatgaaatggttactgtcgaattcagacaatgcggcatttccccaaatgactaagtcgtataggaatcccaggaacaaaccagctctgataatctggggagtgcatcactctggatcagctactgagcagaccaaactctatggaagtggaaacaagttgataacagtaggaagctcgaaataccagcaatcattcactccaagtccgggagcacggccacaagtgaatggacaatcaggaaggattgattttcattggctactccttgaccccaatgacacagtgaccttcactttcaatggggcattcatagcccctgacagggcaagtttctttagaggagaatcgctaggagtccagagtgatgttcctttggattctggttgtgaaggggattgcttccacagtgggggtacgatagtcagttccctgccattccaaaacatcaaccctagaacagtggggaaatgccctcgatatgtcaaacagacaagcctccttttggctacaggaatgagaaacgtcccagagaaccccaagcaggcctaccagaaacggatgaccagaggcctttttggagcgattgctggattcatagagaatggatgggaaggtctcatcgatggatggtatggtttcagacatcaaaatgcacaaggagaaggaactgcagctgactacaaaagcacccaatctgcaatagatcagatcacaggcaaattgaatcgtctgattgacaaaacaaaccagcagtttgaactgatagacaatgaattcagtgagatagaacaacaaatcgggaatgtcattaactggacacgagactcaatgactgaggtatggtcgtataatgctgagctgttggtggcaatggagaatcagcatacaatagatcttgcagactcagaaatgaacaaactttacgaacgcgtcagaaaacaactaagggaaaatgctgaagaagatggaactggatgctttgagatattccataagtgtgatgatcagtgtatggagagcataaggaacaacacttatgaccatacccaatacaggacagagtcattgcagaatagaatacagatagacccagtgaaattgagtagtggatacaaagacataatcttatggtttagcttcggggcatcatgttttcttcttctagccattgcaatgggattggttttcatttgcataaagaatggaaacatgcggtgcactatttgtatatag

SEQ ID NO: 60 - DNA, 1704 bp, Unknown
  Deoptimized Influenza A virus
atgaatacacagatactcgcattcatagcgtgtatgcttatcggaactaaaggcgataaaatttgcttagggcatcacgcagtcgctaacggaactaaagtgaatacgcttaccgaacgcggaatagaggtcgtgaacgctaccgagacagtcgaaacagtcaatataaaaaaaatttgtacacagggaaaaagaccaaccgatctgggacaatgcggactgttagggacactaatcggaccaccacaatgcgatcaattcctcgaattcgacgctaatctgataatcgaacggagagagggaactgacgtatgctatcccggtaagtttacgaacgaagagtcacttagacagatacttagggggtcaggggggatagacaaagagtctatggggtttacatatagcggaatacggactaacggagctacaagtgcatgtagacgatccggatcatcgttttacgccgaaatgaaatggttgttgtctaatagcgataacgctgcattcccacaaatgactaagtcttataggaatcctagaaataaacccgcactgattatttggggagtgcatcatagtggatcagcaaccgaacagactaagttgtacggatcaggtaataaactgattacagtcggatcgagtaaatatcagcaatcgttcacacctagtcccggagctagaccgcaagtgaacggacaatctggtaggattgactttcattggttgcttctagacccaaacgatacagtgacattcacttttaacggagcatttatcgcacccgatagggctagtttctttaggggagagtcactcggagtgcaatcagacgtaccacttgatagcggatgcgaaggcgattgttttcactcagggggaactatagtgagtagtctgccattccaaaatattaatcctagaaccgtcggtaagtgtcctaggtacgttaaacagactagtctattgctcgcaaccggaatgcgtaacgtacccgaaaatcctaaacaggcatatcagaaacggatgactagggggctattcggagcgattgccggattcatagagaatgggtgggagggactgatagacggatggtacgggttcagacaccaaaacgctcagggagagggaacagccgcagactataagtctacgcaatcggcaatcgatcagattaccggtaagcttaatagactgatagacaaaactaatcagcaattcgaactgatagacaacgaatttagtgagatagagcaacagatagggaatgtgataaattggactagagactcaatgactgaggtatggtcatataacgccgaactgttggtcgcaatggagaatcagcatacaatcgatctagccgatagcgaaatgaataaactttacgaaagggtgcgaaaacaattgcgagagaatgcggaagaggacggaaccggatgtttcgaaattttccataaatgcgacgatcaatgtatggaatcgattaggaataatacatacgatcatacacaatatagaaccgaatcacttcagaataggattcaaatcgatcccgttaagttgagtagcggatataaagacattatactatggttctcattcggagctagttgctttctattgcttgcgatagctatgggattggtgttcatatgcataaaaaacggtaatatgcgatgtacgatttgcatatag

SEQ ID NO: 61 - DNA, 1497 bp, Influenza A virus
atggcgtctcaaggcaccaaacgatcttatgaacagatggaaactggtggagaacgccagaatgccactgaaatcagagcatctgttgggagaatggttggtgggatcggaagattctacatacagatgtgcactgaactcaagctcagtgactatgaagggaggctgatccaaaacagcatcacaatagagagaatggttctctcagcatttgatgagaggagaaacaaatatctggaggagcatcccagtgctggaaaagaccctaagaagactggaggtccaatctacaagaggagagatgggaaatggatgagagaattgatcctatatgataaagaggagatcagaaggatttggcgtcaagcgaataatggagaagacgcaactgccggcctcacccatttgatgatctggcactccaatctgaatgatgccacctatcagaggacgagggcacttgtgcgtactggaatggatcccaggatgtgttctctgatgcaaggctcgactcttccgaggaggtctggagctgctggagcagcagtgaaaggagttggaacaatggtgatggaattgatccgaatgatcaagcgagggatcaatgataggaatttctggagaggcgaaaatgggcggagaacaagaattgcttatgagagaatgtgcaacatcctcaaagggaagtttcaaacagcggcacaaagagcgatgatggaccaggtgagggaaagccggaatcctgggaatgctgaaattgaagatctcatatttctcgcacggtctgctctcattctgaggggatcagtggctcataagtcttgcctgcctgcttgtgtgtatggacttgctgtggccagtggatacgactttgaaagggagggatactccctagtcggaatcgatcctttccgtctgctccaaaacagtcaagtcttcagtctcatcagaccaaacgaaaacccagcacataaaagtcagctggtatggatggcatgccactctgcagcttttgaagatctgagagtgtcaagcttcattagaggaacaagagtagtcccaagaggacagctgtccaccagaggagttcagattgcttcaaatgagaacatggagacaatggactccagtactcttgaactgaggagcagatactgggctataaggaccagaagtggaggaaacactaaccagcagagagcatccgcagggcagatcagcgtacagcccacattctctgtacagaggaacctcccattcgagagagcaaccattatggcggcatttacagggaacactgaaggcagaacttcagacatgagaacagaaatcataaggatgatggaaaatgccagacctgaggatgtgtctttccaggggcggggagtcttcgagctctcggacgaaaaggcaacgaacccgatcgtgccttcctttgacatgagtaacgaaggatcttatttcttcggagacaatgcagaggagtatgacaattaa

SEQ ID NO: 62 - DNA, 1497 bp, Unknown
  Deoptimized Influenza A virus
atggctagtcagggaacgaaacggtcttacgaacagatggagacagggggagagagacagaacgctaccgaaattagggctagtgtgggaagaatggtcgggggaatcggtaggttctatatacagatgtgtaccgaactcaaactgtccgattacgaagggagattgatccaaaactcaatcacaatcgaacgtatggtgcttagcgcattcgacgaaagacgaaataagtatctcgaagagcatcctagcgcaggtaaggacccaaaaaagacaggcggaccaatctataaacgtagggacggaaaatggatgagggaactgatactgtatgataaggaggagatcagacggatttggagacaggctaataacggcgaagacgcaaccgcaggactgacacaccttatgatttggcactctaatctgaacgacgctacatatcaacggactagagctctcgttagaaccggaatggaccctagaatgtgtagtctgatgcagggatctacactccctaggagatctggcgcagccggagcggcagttaagggagtcggaactatggtaatggagttgatcagaatgatcaaaagggggattaacgatagaaatttttggaggggcgaaaacggaaggcgaactaggatcgcatacgaacgtatgtgcaatatccttaagggaaagtttcagactgccgcacagagagctatgatggatcaggttagggagtctaggaatcccggtaacgccgaaatcgaagatctgatctttctcgctagatccgcactcatactcagagggtccgttgcgcataagtcttgcctacccgcatgcgtatacggactcgcagtcgctagcggatacgatttcgaacgagaggggtatagtctcgtcggaatcgatccatttaggttgctccagaatagtcaggtgtttagtctgattagaccgaacgagaatcctgcacataaatcgcaactcgtttggatggcatgccatagcgcagcattcgaagaccttagagtgtcatctttcatacgcggaactagggtagtgcctagggggcaactgtctactaggggggtgcaaatcgctagtaacgagaatatggagactatggactctagtacactcgaactgagatctaggtattgggcaatcagaactagatccggagggaatacgaatcagcaaagagcgtcagccggacagatatccgtgcaacctacattctcagtgcaacggaatctgccattcgaaagagcgactattatggccgcattcacagggaataccgaagggagaactagcgatatgagaaccgagattatcagaatgatggagaacgctagacccgaagacgtgagttttcagggaaggggagtgttcgaactatccgacgaaaaagcgactaacccaatcgtaccgtcattcgatatgtctaacgagggatcgtattttttcggcgataacgctgaagagtatgacaattaa

SEQ ID NO: 63 - DNA, 1410 bp, Influenza A virus
atgaatccgaatcagaagataataacaatcggggtagtgaataccactctgtcaacaatagcccttctcattggagtgggaaacttagttttcaacacagtcatacatgagaaaataggagaccatcaaatagtgacccatccaacaataatgacccctgaagtaccgaactgcagtgacactataataacatacaataacactgttataaacaacataacaacaacaataataactgaagcagaaaggcctttcaagtctccactaccgctgtgccccttcagaggattcttcccttttcacaaggacaatgcaatacgactgggtgaaaacaaagacgtcatagtcacaagggagccttatgttagctgcgataatgacaactgctggtcctttgctctcgcacaaggagcattgctagggactaaacatagcaatgggaccattaaagacagaacaccatataggtctctaattcgtttcccaataggaacagctccagtactaggaaattacaaagagatatgcattgcttggtcgagcagcagttgctttgacgggaaagagtggatgcatgtgtgcatgacagggaatgataatgatgcaagtgcccagataatatatggaggaagaatgacagactccattaaatcatggaggaaagacatactaagaacccaggagtctgaatgtcaatgcattgacgggacttgtgttgttgctgtcacagatggccctgctgctaatagtgcagatcacagggtttactggatacgggagggaagaataataaagtatgaaaatgttcccaaaacaaagatacaacacttagaagaatgttcctgctatgtggacattgatgtttactgtatatgtagggacaattggaagggctctaacagaccttggatgagaatcaacaacgagactatactggaaacaggatatgtatgtagtaaatttcactcagacacccccaggccagctgacccttcaataatgtcatgtgactccccaagcaatgtcaatggaggacccggagtgaaggggtttggtttcaaagctggcaatgatgtatggttaggtagaacagtgtcaactagtggtagatcgggctttgaaattatcaaagttacagaagggtggatcaactctcctaaccatgtcaaatcaattacacaaacactagtgtccaacaatgactggtcaggctattcaggtagcttcattgtcaaagccaaggactgttttcagccctgtttttatgttgagcttatacgagggaggcccaacaagaatgatgacgtctcttggacaagtaatagtatagttactttctgtggactagacaatgaacctggatcgggaaattggccagatggttctaacattgggtttatgcccaagtaa

SEQ ID NO: 64 - DNA, 1410 bp, Unknown
  Deoptimized Influenza A virus
atgaatcctaatcagaaaataattactataggggtcgttaatactacactatctacaatcgctctactaatcggagtcggtaatctagtctttaatacagtgatacacgaaaagataggcgaccatcagatagtgacacatcctacaattatgacacccgaagtgcctaattgtagcgatacaataattacatataacaataccgttataaacaatattacaacaacaattataaccgaagccgaacgaccattcaaaagtccactacccctatgtccatttagggggttttttccgtttcataaggataacgctatacggttaggcgaaaataaagacgtaatcgttactagggagccatacgttagttgcgataacgataattgttggtcattcgcactcgctcaaggcgcactgttagggactaaacactctaacggaacaattaaagacagaacaccttataggtcactgataagattccctatcggaaccgctcccgtactaggcaattataaagagatatgcatagcatggtcaagttcgtcatgtttcgacggtaaagagtggatgcacgtatgtatgaccggtaacgataacgacgctagcgcacagataatatacggagggcgaatgacagactcaattaagagttggcgtaaagacatactgagaacacaagagtccgaatgccaatgcatagacggaacttgcgtagtcgccgttacagacggacccgcagctaactccgctgaccatagagtgtattggattagggagggaaggataataaagtatgagaacgtgcctaagactaagatacaacatcttgaagagtgttcatgttatgtcgacatagacgtgtattgcatatgtagagacaattggaaagggtctaataggccatggatgagaataaataacgaaactatactcgaaaccggatacgtatgttctaagttccatagcgatacacctagacccgcagacccatctattatgtcatgcgatagcccatctaacgttaacggcggacccggagtcaaagggttcggattcaaagccggtaacgacgtttggttagggagaaccgttagtactagcggtaggtccggattcgaaattataaaggttacagaggggtggataaatagtccgaatcacgttaagtcaattacacaaacacttgtgtctaataacgattggtccggatatagcggatcattcatagtcaaagctaaggattgctttcagccatgtttttacgtcgaactgataagggggagaccgaataaaaacgacgacgttagttggactagtaattcgatagtgacattttgcggattggacaacgaacccggatccggtaattggcctgacggatcgaatatagggtttatgcctaaataa

SEQ ID NO: 65 - DNA, 2274 bp, Influenza A virus
atggatgtcaatccgactttacttttcttaaaagtgccagcgcaaaatgctataagtactacattcccttatactggagatcctccatacagccatggaacaggaacaggatacaccatggacacagtcaacagaacacatcaatactcagagaaggggaggtggacaacaaacacagagactggagcaccccaactcaacccaattgatggaccattacctgaggacaacgagccaagcggatatgcacaaacagattgcgtgttggaagcaatggctttccttgaagaatcccacccagggatctttgaaaactcttgtcttgaaacgatggaagtcgttcagcaaacaagagtggacaaactaacccaaggtcgccagacttatgactggacactgaatagaaaccagccagctgcaactgccttggccaacactatagaggttttcagatcgaacggtctgacagccaatgaatcggggagactaatagatttcctcaaggatgtaatggaatcaatggataaagaagaaatggaaataacaacacatttccagagaaagagaagagtaagggacaacatgaccaagaaaatggtcacacaaagaacaatagggaagaagaagcagaggctgaacaagaggagctatttaataagagcactgacattgaacacaatgacaaaggatgcagaaagaggcaaattgaagaggcgggcaattgcaacacccgggatgcagattagaggattcgtgtactttgtcgaaacactggcgaggagcatctgtgagaaacttgagcaatctggacttcccgttggggggaatgagaagaaggctaaattggcaaatgtcgtgagaaaaatgatgactaattcacaagacacagagctctcctttacaattactggagacaacaccaaatggaatgagaatcaaaatcctcggatgtttctggcaatgataacatacatcacaagaaaccaacctgagtggtttagaaatgtcttgagcattgcccccataatgttctcaaacaaaatggcaaggttaggaaaaggatacatgtttgagagtaagagcatgaagctacggacacaaataccagcagaaatgcttgcaaacattgacctgaaatacttcaacgaatcaacgagaaagaaaatcgagaaaataagacctctgctaatagatggcacagcctcattgagtcctggaatgatgatgggcatgttcaacatgctgagtacagtattaggagtttcaatcctgaatcttggacaaaagaggtacaccaaaactacatactggtgggatgggctccaatcctctgatgatttcgctctcatagtgaatgcaccgaatcatgagggaatacaagcgggagtggataggttctataggacctgcaaactggttggaatcaacatgagcaaaaagaagtcttatataaaccggacgggaacatttgagttcacaagctttttctaccgctatggatttgtagccaacttcagtatggaattgcccagcttcggagtgtctggaatcaatgaatcggctgacatgagcattggggttacagtgataaagaacaatatgataaacaatgaccttggaccagcaacagctcagatggctcttcagctattcatcaaggactacaggtacacataccgatgccacaggggtgatacacaaattcaaacaagaagatcattcgagctgaagaagctgtgggagcagacccgttcaaaggcaggactgttggtatcagatggaggaccaaacctatacaacatccggaatctccacatcccagaggtctgcttgaagtgggaactaatggatgaagattaccagggcaggctgtgtaaccctctgaatccgtttgtcagtcataaggaaattgaatccgtaaacaatgctgtggtaatgccagctcatggcccggccaagagcatggaatatgatgccgttgcgactacacactcatggatccctaagaggaatcgttccattctcaataccagccaaaggggaattcttgaggatgagcagatgtaccagaagtgctgcaacctatttgagaaattcttccccagtagttcatacaggaggccagttggaatttccagcatggtggaggccatggtgtctagggcccgaattgatgcacgcattgatttcgaatctggaaggatcaagaaagaagagtttgctgagatcatgaagatctgttccaccattgaagagctcagacggcaaaaatag

SEQ ID NO: 66 - DNA, 2274 bp, Unknown
  Deoptimized Influenza A virus
atggacgttaaccctacactgttattccttaaggtgccagcacaaaacgcaattagtacaacattcccatacacaggcgatccaccatactcacacggaaccggaaccggatacactatggataccgttaatagaacacaccaatactccgaaaagggaaggtggacaacgaatacagagacaggcgcaccacaactgaatccgatagacggaccactgccagaggataacgaacctagcggatacgcacagaccgattgcgtactcgaagcgatggcatttctcgaagagtcacatcccggaattttcgaaaacagttgcctcgagactatggaggtcgtgcaacagactagggtcgataagttgacacaggggagacagacatacgattggacactgaatagaaaccaacctgccgcaaccgcactcgctaacacaatcgaagtgtttaggtctaacggattgaccgctaacgagtccggaaggttgatcgatttccttaaggacgtaatggagtcaatggataaggaggagatggagattacaacacatttccaacggaaaagacgcgttagggataatatgactaaaaagatggtgacacaacggacaatcggtaagaaaaaacagagactgaataagagatcgtatctgattagggcacttacattgaatacaatgacaaaagacgccgaacgcggaaagttgaaacgtagagccatagccactcctggaatgcaaatacgcggattcgtatacttcgtcgagacactcgctaggtcaatttgcgaaaagctagagcaatccggactaccagtcggagggaacgaaaagaaagcgaaactcgctaacgtagtgagaaaaatgatgacaaactcacaggatacagagttaagctttacaattacaggcgataatacgaaatggaacgagaatcagaatcctagaatgtttctggcaatgataacatacataacacgtaaccaacccgaatggtttagaaacgtactgtcaatcgcacctattatgttttcgaataagatggctagattgggtaaggggtatatgttcgaaagtaagtctatgaagcttaggacacagatacccgccgaaatgttggcgaatatcgatcttaagtactttaacgaatcgacacgaaaaaagatcgaaaagattaggccactgttaatcgacggaaccgctagcctatcccccggtatgatgatggggatgtttaatatgctatcaacagtgttaggcgttagcatactgaacctagggcaaaaaaggtacactaagactacttattggtgggacggactgcaatctagcgacgatttcgcactaatcgttaacgcaccgaatcacgaagggatacaggccggagtcgataggttctatagaacatgcaagttagtcggaattaacatgtctaaaaaaaagtcatacattaacagaaccggaacattcgaattcacaagctttttttacaggtacggattcgtcgctaactttagcatggagttacctagtttcggagtgagcggaattaacgaatccgccgatatgtcaatcggagtgacagtgataaagaataatatgattaataacgatctcggacctgcaaccgcacaaatggcattgcaattgttcataaaggattataggtatacatataggtgccataggggcgatacacagatacagactagacgatcattcgaactgaaaaaactgtgggagcaaactaggtctaaggccggattgctagttagcgacggagggccaaacctatacaatatacggaatctgcatatacccgaagtgtgtcttaagtgggagcttatggacgaggattaccaggggagactgtgtaacccacttaacccattcgttagccataaggagatcgaatccgttaataacgccgtagtgatgcctgcacacggacccgctaagagtatggagtacgatgccgtagcgacaacgcatagttggataccgaaacggaataggtctatccttaacactagccaaagggggatactcgaagacgaacaaatgtatcaaaagtgttgcaatctgttcgaaaagttctttccgtctagctcatacagaagaccagtcggaattagctcaatggtcgaggctatggtgagtagggctagaatcgacgctagaatcgatttcgaatccggacggattaagaaagaggagttcgcagagataatgaaaatttgtagtacaatcgaagagcttagacggcaaaaatag

SEQ ID NO: 67 - DNA, 1710 bp, Influenza A virus
agcaaaagcaggggatacaaaatgaacactcaaatcctggtattcgctctggtggcgagcattccgacaaatgcagacaagatctgccttgggcatcatgccgtgtcaaacgggactaaagtaaacacattaactgagagaggagtggaagtcgttaatgcaactgaaacggtggaacgaacaaacgttcccaggatctgctcaaaagggaaaaggacagttgacctcggtcaatgtggacttctgggaacaatcactgggccaccccaatgtgaccaattcctagaattttcggccgacttaattattgagaggcgagaaggaagtgatgtctgttatcctgggaaattcgtgaatgaagaagctctgaggcaaattctcagagagtcaggcggaattgacaaggagacaatgggattcacctacagcggaataagaactaatggaacaaccagtgcatgtaggagatcaggatcttcattctatgcagagatgaaatggctcctgtcaaacacagacaatgctgctttcccgcaaatgactaagtcatacaagaacacaaggaaagacccagctctgataatatgggggatccaccattccggatcaactacagaacagaccaagctatatgggagtggaaacaaactgataacagttgggagttctaattaccaacagtcctttgtaccgagtccaggagcgagaccacaagtgaatggccaatctggaagaattgactttcattggctgatactaaaccctaatgacacggtcactttcagtttcaatggggccttcatagctccagaccgtgcaagctttctgagagggaagtccatgggaattcagagtgaagtacaggttgatgccaattgtgaaggagattgctatcatagtggagggacaataataagtaatttgccctttcagaacataaatagcagggcagtaggaaaatgtccgagatatgttaagcaagagagtctgctgttggcaacaggaatgaagaatgttcccgaaatcccaaagaggaggaggagaggcctatttggtgctatagcgggtttcattgaaaatggatgggaaggtttgattgatgggtggtatggcttcaggcatcaaaatgcacaaggggagggaactgctgcagattacaaaagcacccaatcagcaattgatcaaataacagggaaattaaatcggcttatagaaaaaactaaccaacagtttgagttaatagacaacgaattcactgaggttgaaaggcaaattggcaatgtgataaactggaccagagattccatgacagaagtgtggtcctataacgctgaactcttagtagcaatggagaatcagcacacaattgatctggccgactcagaaatgaacaaactgtacgaacgagtgaagagacaactgagagagaatgccgaagaagatggcactggttgcttcgaaatatttcacaagtgtgatgacgactgcatggccagtattagaaacaacacctatgatcacagcaagtacagggaagaagcaatacaaaatagaatacagattgacccagtcaaactaagcagcggctacaaagatgtgatactttggtttagcttcggggcatcatgtttcatacttctggccattgcaatgggccttgtcttcatatgtgtgaagaatggaaacatgcggtgcactatttgtatataa

SEQ ID NO: 68 - DNA, 1710 bp, Unknown
  Deoptimized Influenza A virus
agtaagagtagggggtataaaatgaatacacagatactcgtattcgcactcgttgcgtcaataccgacaaacgccgataagatttgcctagggcatcacgcagtgtcaaacggaactaaagtgaatacacttaccgaaaggggcgttgaggtagtgaacgctacagagactgtcgaacggactaacgtacctaggatttgtagtaagggtaaaagaacagtcgacctagggcaatgcggactgttaggcacaattaccggaccaccacaatgcgaccaatttctcgaatttagcgctgatctgattatcgaacggagagagggatccgacgtttgttatcccggtaaattcgttaacgaagaggcactgagacagatacttagagaatccggagggatagacaaagagacaatggggtttacatatagcggaattagaactaacggaactactagcgcatgtaggagatccggatctagcttttacgccgaaatgaaatggttactgtcaaataccgataacgccgcatttccgcaaatgactaagtcatataagaatactaggaaagaccccgcactgataatttgggggatacaccatagcggatcgactaccgaacagacaaagctatacggtagcgggaataaactgataacagtgggatcaagtaattaccaacagtcattcgtaccgagtccaggcgctagaccacaagtgaacggacaatccggacgtatagatttccattggttgatactgaatccgaacgatacagtgacatttagctttaacggcgcattcatagcacccgatagggcatcattccttaggggtaagagtatggggatacaaagcgaagtgcaagtcgacgctaattgcgaaggcgattgttatcatagcggggggactattattagtaatctgccattccaaaatattaatagtagggcagtgggaaagtgtccaaggtacgttaaacaggaatcactgttactcgcaaccggaatgaaaaacgtaccagagatacctaagagacgaagaagggggttgttcggcgctatagccggattcatagagaacggatgggagggactgatagacggatggtacgggttcagacaccaaaacgctcaaggcgaagggacagccgcagactataagagtacacaatccgctatcgatcaaattaccggtaagcttaatagactgatcgaaaaaactaatcaacaattcgaactaatcgataacgaatttacggaagtcgaaagacagattggcaatgtgataaattggactagagactctatgactgaggtttggtcatataacgccgaactgttagtcgcaatggaaaatcagcatacgatagaccttgccgatagcgaaatgaataagctatacgaaagggtgaaacgacaattgagggaaaacgccgaagaggacggaacagggtgtttcgaaatttttcacaaatgcgacgacgattgtatggctagtattaggaataatacatacgaccatagtaagtatagagaggaagcgatacagaataggattcaaatcgatcccgtaaaactgtctagcggatacaaagacgttatactgtggttctcattcggagcgtcatgtttcatactgcttgcaatcgctatggggttagtgttcatatgcgttaaaaacggaaatatgcgatgtactatttgtatttaa

SEQ ID NO: 69 - DNA, 1497 bp, Influenza A virus
atggcgtctcaaggcaccaaacgatcttatgaacagatggaaactggtggggaacgccagaatgctactgagatcagagcatctgtcggaagaatggttggtggaattgggaggttttacatacagatgtgcacagaactcaaactcagcgaccatgaagggaggctgatccagaacagcataacaatagagagaatggttctctctgcatttgatgaaagaaggaacaaatacctggaagaacatcccagtgcggggaaggacccgaagaaaactggaggtccaatctaccgaaggagagacgggaaatggatgagggagttgattctgtatgacaaagaggagatcaggaggatctggcgtcaagcaaacaacggagaagacgcaactgctggtctcactcatttgatgatctggcattccaacctgaatgatgccacatatcagagaacgagagctctcgtgcgcactggtatggacccaagaatgtgctctctgatgcaaggatcaaccctcccgaggagatctggagctgctggtgcagcagtaaaaggagtcgggacgatggtgatggaactaattcggatgataaagcgagggattaacgataggaatttctggagaggcgaaaacggaaggaggacaagaattgcatatgagagaatgtgcaacatcctcaaagggaaattccaaacagcagcacaaagagcaatgatggatcaagtacgagaaagcagaaatcctgggaatgctgaaattgaagatctcatctttctggcacggtctgcactcatcctgagaggatcagtggcccataagtcctgcttgcctgcttgtgtgtacggacttgctgtggccagtggatatgactttgagagagaaggatactctctggtcggaatagatcctttccgtcttctccaaaacagccaggtcttcagtctcattagaccaaatgagaatccagcacacaagagtcaactggtatggatggcatgccattctgcagcgtttgaagacctgagagtatcaagtttcatcagagggacaagagtggttccaagaggacaactatccaccagaggagttcaaattgcttcaaatgagaacatggaaacaatggactccagtactctcgaactgagaagcagatattgggctataaggaccaggagtggaggaaacaccaaccaacagagagcatctgcaggacaaatcagtgtacaacctaccttctcagtacagagaaatcttccctttgaaagggcgaccattatggcggcatttacagggaacactgagggcagaacatctgacatgaggactgaaatcataagaatgatggaaagtgccagaccagaagatgtgtctttccaggggcggggagtcttcgagctctcggacgaaaaggcaacgaacccgatcgtgccttcctttgacatgagtaatgaaggatcttatttcttcggagacaatgcagaggagtatgacaattaa

SEQ ID NO: 70 - DNA, 1497 bp, Unknown
  Deoptimized Influenza A virus
atggctagtcagggaactaagagatcatacgaacagatggaaaccggaggcgaacgacaaaacgcaaccgaaatcagggctagcgtcggaaggatggtagggggaatcggaagattctatatccaaatgtgtacggaactcaaattgtccgatcacgaaggtagactgatacagaattcgattacaatcgagagaatggtgcttagcgcattcgacgaaagacgtaataagtatctcgaagagcatccatccgcaggtaaggacccaaaaaaaaccggagggccaatctatagaaggagagacggtaagtggatgcgcgaactcatactgtatgacaaagaggagattagacggatttggcgacaagcgaataacggagaggacgctacagccggattgacacatctgatgatatggcattctaatctgaacgacgctacttatcaacgaactagggcactcgttaggaccggtatggaccctagaatgtgctctcttatgcaggggtctacactccctagacggtctggcgctgccggagccgcagttaaaggagtcggaactatggttatggaactgattagaatgattaaaagggggattaacgatagaaacttttggagaggcgaaaacggtaggcgaactagaatcgcatacgaaaggatgtgcaatatactcaaaggtaagtttcagaccgcagcgcaacgcgctatgatggaccaagtgagagagtctaggaatcccggaaacgctgagatcgaagacctaatctttctcgctagatccgcactgatactgagagggtcagtcgcacataagtcttgcctaccagcatgcgtttacggactcgcagtcgcaagcggatacgatttcgaaagagaggggtatagtctcgtcggaatcgatccgtttagattgctccaaaatagtcaggtgtttagtctgataagacctaacgagaatcccgcacataaatctcaactcgtatggatggcatgccatagcgcagcattcgaagaccttagagtgagtagttttattagggggactagggtagtgcctagggggcaactgtctactaggggggtgcaaatcgctagtaacgaaaacatggagactatggactcttctacactcgaactcagatctagatattgggcaatcagaacgagatccggagggaatacgaatcaacagagagcgagtgcaggacagattagtgtgcaaccgacattctcagtgcaacggaatctgccattcgaaagagcgacaattatggccgcattcacagggaataccgaagggagaacaagcgatatgagaaccgaaatcatacgtatgatggaatccgctaggccagaggacgtaagttttcagggaaggggagtattcgaactgtctgacgaaaaagcgactaaccctatcgtaccgtcattcgatatgtctaacgagggatcatattttttcggagacaacgcagaggaatacgataactaa

SEQ ID NO: 71 - DNA, 1416 bp, Influenza A virus
atgaatccaaatcagaaactatttgcattatctggagtggcaatagcacttagtgtactgaacttattgataggaatctcaaacgtcggattgaacgtatctctacatctaaaggaaaaaggacccaaacaggaggagaatttaacatgcacgaccattaatcaaaacaacactactgtagtagaaaacacatatgtaaataatacaacaataattaccaagggaactgatttgaaaacaccaagctatctgctgttgaacaagagcctgtgcaatgttgaagggtgggtcgtgatagcaaaagacaatgcagtaagatttggggaaagtgaacaaatcattgttaccagggagccatatgtatcatgcgacccaacaggatgcaaaatgtatgccttgcaccaagggactaccattaggaacaaacattcaaatggaacgattcatgacagaacagctttcagaggtctcatctccactccattgggcactccaccaaccgtaagtaacagtgactttatgtgtgttggatggtcaagcacaacttgccatgatgggattgctaggatgactatctgtatacaaggaaataatgacaatgctacagcaacggtttattacaacagaaggctgaccactaccattaagacctgggccagaaacattctgaggactcaagaatcagaatgtgtgtgccacaatggcacatgtgcagttgtaatgaccgacggatcggctagtagtcaagcctatacaaaagtaatgtatttccacaagggattagtagttaaggaggaggagttaaggggatcagccagacatattgaggaatgctcctgttatggacacaatcaaaaggtgacctgtgtgtgcagagataactggcagggagcaaacaggcctattatagaaattgatatgagcacattggagcacacaagtagatacgtgtgcactggaattctcacagacaccagcagacctggggacaaatctagtggtgattgttccaatccaataactgggagtcccggcgttccgggagtgaagggattcgggtttctaaatggggataacacatggcttggtaggaccatcagccccagatcaagaagtggattcgaaatgttgaaaatacctaatgcaggtactgatcccaattctagaatagcagaacgacaggaaattgtcgacaataacaattggtcaggctattccggaagctttattgactattggaatgataacagtgaatgctacaatccatgcttttacgtagagttaattagaggaagacccgaagaggctaaatacgtatggtgggcaagtaacagtctaattgccctatgtggaagcccattcccagttgggtctggttccttccccgatggggcacaaatccaatacttttcgtaa

SEQ ID NO: 72 - DNA, 1416 bp, Unknown
  Deoptimized Influenza A virus
atgaatccgaaccaaaaattgttcgcattaagcggagtcgcaatcgcactaagcgtactgaatctgttgatagggataagtaacgtagggttgaacgtatcactacatttgaaagagaaagggcctaaacaggaagagaatttgacatgtactacaattaatcagaataatactaccgtagtcgaaaatacatacgttaacaatacaacaattattactaagggaaccgatctgaaaactccaagttatctgttactgaataaatctctatgtaacgttgagggatgggtagtgatcgcaaaggataacgccgttagattcggcgaaagcgaacagattatagtgactagagagccatacgtatcatgcgatccaaccggatgcaaaatgtacgcattacaccaagggacaactattaggaataaacactctaacggtacgatacacgatagaaccgcatttagggggttgattagtacaccactcggtacaccaccaaccgtttcgaatagcgactttatgtgcgtagggtggtctagtactacatgtcacgacggaatcgctagaatgacaatttgcatacaggggaataacgataacgctaccgcaaccgtatattataatagaagactaactactactattaagacatgggctaggaatatactgagaacgcaagaatccgaatgcgtttgtcataacggtacatgcgccgtagtgatgaccgacggatccgctagttcgcaagcatatactaaggtaatgtattttcacaaagggttagtagtgaaagaggaagagttgagggggtccgctagacatattgaggaatgctcatgttacggacataatcaaaaggtgacatgcgtatgtagagacaattggcaaggcgcaaatagacccattatcgaaatcgatatgagtacactcgaacatactagtagatatgtgtgtaccggaatactaaccgatacgagtagacccggcgataagtctagcggagattgctcaaacccaattaccggatcacccggagtgccaggcgttaagggattcggattccttaacggagacaatacatggttagggagaactattagtcctaggagtaggtccggattcgaaatgcttaagatacctaacgccggaaccgacccaaatagtaggattgccgaacgacaagagattgtcgacaataacaattggtccggatatagcggatcattcatagactattggaacgacaatagcgaatgctataacccatgtttttacgttgagttgattaggggtagacccgaagaggcaaaatacgtttggtgggcatctaacagtctaatcgcattatgcggatcaccatttcccgtaggtagcggatcatttcccgacggagcccaaattcaatattttagttaa

SEQ ID NO: 73 - DNA, 2277 bp, Influenza A virus
atggatgtcaatccgactttacttttcttaaaagtgccagcgcaaaatgcaataagtaccacattcccttatactggagatcccccatatagccatggaacaggaacaggatacaccatggacacagtcaacagaacacatcaatattcagaaaaagggaggtggacaacaaacacagagaccggagcaccccaactcaaccctattgatggaccattacctgaagacaatgagccgagcgggtatgcacaaacagattgtgtattggaagcaatggctttccttgaagaatcccacccaggactctttgaaaactcatgtcttgaaacgatggaagttgtccagcaaacgagagtggataagctgacccaaggtcgccagacttatgactggacattgaatagaaaccagccggctgcaactgctttggccaacaccatagaagtattcagatcgaacggtctaacagccaatgagtcaggaaggttaatagatttcctcaaggacgtaatggaatcaatggataaggaagaaatggaaataacaacacatttccagagaaagagaagagtgagggacaacatgaccaagaaaatggtcacacaaagaacaatagggaagaagaagcaaaagctgacaaaaaagagctacctaataagagcactgacactgaacacaatgacaaaagatgctgaaaggggaaaattgaaaagacgagcgattgcaacacccggaatgcaaatcagaggattcgtgcactttgtcgaagcactagcaaggagcatctgtgaaaaacttgagcaatctggactccccgttggagggaatgagaagaaggctaaattggcaaatgttgtgagaaagatgatgactaactcacaagacacagagctctcctttacagttaccggagacaacaccaaatggaatgagaatcagaatcctcgaatatttctagcaatgataacatacatcacaaggaaccaacctgaatggtttagaaatgtcttgagcattgcccctataatgttctcaaataaaatggcgaggttaggaaaaggatacatgttcgagagtaagagcatgaagctacggacacaaataccagcagaaatgcttgcaaacattgacttgaaatacttcaacgaatcgacgagaaagaaaattgagaaaataagacctctactaatagagggcacagcctcattgagtccagggatgatgatgggcatgtttaatatgctaagtacggtcttaggagtctcaatcttaaatcttgggcagaagaggtacaccaaaaccacatactggtgggatgggctccaatcctctgatgatttcgctctcatagtgaatgcaccaaatcatgagggaatacaagcaggagtggatagattctataggacttgcaagctagttggaatcaacatgagcaaaaagaagtcttacataaatcggacaggaacatttgagttcacaagctttttctaccgctatgggtttgtagccaacttcagcatggagctgcccagctttggagtttccggaattaatgaatcggctgacatgagcattggagttacagtgataaagaataatatgataaacaacgaccttggaccagcaacagcccagatggctcttcagctgttcattaaagactacagatacacctaccgatgccacagaggtgatacacaaattcaaactagaagatcatttgaattgaagaagctgtgggagcagacccgctcaaaggcaggactgttggtttcagatggagggccgaatttatacaacatccggaatcttcacattccagaagtttgcttgaagtgggagttgatggatgaagattaccagggaagactgtgtaaccctctgaacccgtttgtcagtcataaggaagttgaatccgtcaacaatgctgtggtaatgccagcccatggtccggccaagagcatggaatatgatgccgttgcaactacacattcatggattcccaagagaaatcgctccattctcaacactagccaaaggggaattcttgaggatgaacaaatgtaccagaagtgctgcactctattcgagaaattcttccctagcagttcatatcggaggccagttggaatttccagcatgatggaggccatggtgtctagggcccgaattgatgcacggattgacttcgagtctggaaggattaagaaagaagaatttgctgagatcttgaagatctgttccaccattgaagagctcggacggcaagggaagtga

SEQ ID NO: 74 - DNA, 2277 bp, Unknown
  Deoptimized Influenza A virus
atggacgttaaccctacactgttgttccttaaggtacccgctcaaaacgctataagcacaacatttccatataccggagatccgccatactcacacggaaccggaaccggatacacaatggataccgttaataggacacaccaatatagcgaaaagggaaggtggactacgaataccgaaaccggagcaccacaattgaatccgatagacggaccgttacccgaggataacgaacctagcggatacgcccaaaccgattgcgtactcgaggctatggcctttctcgaagagtcacatcccggactattcgaaaactcatgtctcgaaacaatggaggtcgtgcaacagactagggtcgataagcttacacaagggcgacagacatacgattggacactgaatagaaaccaacctgccgcaaccgcactagcgaatacaatagaggtgtttagatctaacggattgaccgcaaacgaatccggacggttgatcgatttccttaaggacgtaatggagtcaatggataaagaggagatggagattacgacacatttccaacgaaaaagacgcgttagggataacatgacaaaaaagatggtgacacaacggactatcggtaagaaaaaacaaaaacttacgaaaaaatcttatctgatacgcgcactaacacttaatacaatgactaaggacgccgaacgcggtaagcttaagagacgcgcaatcgcaacacccggaatgcaaattagggggttcgtgcatttcgttgaggcactcgctagatcgatttgcgaaaagctcgagcaatccggactgccagtcggggggaacgaaaaaaaggctaagttagcgaacgttgtgagaaaaatgatgactaatagccaggatacagagttaagctttaccgttaccggagataatactaagtggaacgagaatcagaatcctagaatattcttggcaatgattacatacattacacgaaatcaaccggaatggtttagaaacgtattgagcatagcccctattatgttttcgaacaaaatggcaagattgggtaaggggtatatgttcgaatcgaagtctatgaaattgagaacacagataccagccgaaatgcttgcgaatatcgatcttaagtactttaacgaatcaactaggaaaaaaatcgaaaagattagaccactactgatagagggaacagcaagcctatcccccggaatgatgatggggatgtttaatatgttgagcacagtgttaggcgttagcatactgaatctcggacagaagagatacactaagacaacatattggtgggacggattgcaatctagcgacgatttcgcactgatagttaacgcacctaaccacgaagggatacaggccggagtcgataggttctatagaacatgtaagttagtcggaattaatatgagtaagaaaaagtcatacattaatagaaccggaacattcgaattcacaagctttttttacagatacggattcgtcgctaactttagtatggagttgccatcattcggagtgtccggaattaacgaatccgccgatatgtcaatcggagtgacagtgattaaaaacaatatgattaataacgatctcggaccagcaaccgcacaaatggcactgcaattgttcattaaggattacagatatacatacagatgccataggggcgatacacagatacagacaagacggtcattcgaattgaaaaagttatgggagcaaactaggagtaaggccggactactcgttagcgacggagggcctaacctatacaatattaggaatctacatatacccgaagtgtgtcttaagtgggagcttatggacgaagactatcagggacggttatgcaatccactaaacccattcgttagccataaggaggtcgaatccgttaataacgccgtcgttatgcctgcacacggacctgctaagtctatggaatacgacgcagtcgcaactacacatagttggataccgaaacggaatagatccatactgaatacgagtcaaagggggatactcgaagacgaacaaatgtatcaaaagtgttgtacactattcgaaaagttttttccgtcaagctcatacagacgaccagtcggaattagctcaatgatggaggctatggtaagtagggctaggatagacgctagaatcgatttcgaatccggacggattaagaaagaggaattcgccgaaattctgaaaatttgctcaacaatcgaagagttagggagacagggtaagtga

SEQ ID NO: 75 - DNA, 1683 bp, Influenza A virus
atggaaacaatatcactaataactatactactagtagtaacagcaagcaatgcagataaaatctgcatcggccaccagtcaacaaactccacagaaactgtggacacgctaacagaaaccaatgttcctgtgacacatgccaaagaattgctccacacagagcataatggaatgctgtgtgcaacaagcctgggacatcccctcattctagacacatgcactattgaaggactagtctatggcaacccttcttgtgacctgctgttgggaggaagagaatggtcctacatcgtcgaaagatcatcagctgtaaatggaacgtgttaccctgggaatgtagaaaacctagaggaactcaggacactttttagttccgctagttcctaccaaagaatccaaatcttcccagacacaacctggaatgtgacttacactggaacaagcagagcatgttcaggttcattctacaggagtatgagatggctgactcaaaagagcggtttttaccctgttcaagacgcccaatacacaaataacaggggaaagagcattcttttcgtgtggggcatacatcacccacccacctataccgagcaaacaaatttgtacataagaaacgacacaacaacaagcgtgacaacagaagatttgaataggaccttcaaaccagtgatagggccaaggccccttgtcaatggtctgcagggaagaattgattattattggtcggtactaaaaccaggccaaacattgcgagtacgatccaatgggaatctaattgctccatggtatggacacgttctttcaggagggagccatggaagaatcctgaagactgatttaaaaggtggtaattgtgtagtgcaatgtcagactgaaaaaggtggcttaaacagtacattgccattccacaatatcagtaaatatgcatttggaacctgccccaaatatgtaagagttaatagtctcaaactggcagtcggtctgaggaacgtgcctgctagatcaagtagaggactatttggagccatagctggattcatagaaggaggttggccaggactagtcgctggctggtatggtttccagcattcaaatgatcaaggggttggtatggctgcagatagggattcaactcaaaaggcaattgataaaataacatccaaggtgaataatatagtcgacaagatgaacaagcaatatgaaataattgatcatgaattcagtgaggttgaaactagactcaatatgatcaataataagattgatgaccaaatacaagacgtatgggcatataatgcagaattgctagtactacttgaaaatcaaaaaacactcgatgagcatgatgcgaacgtgaacaatctatataacaaggtgaagagggcactgggctccaatgctatggaagatgggaaaggctgtttcgagctataccataaatgtgatgatcagtgcatggaaacaattcggaacgggacctataataggagaaagtatagagaggaatcaagactagaaaggcagaaaatagagggggttaagctggaatctgagggaacttacaaaatcctcaccatttattcgactgtcgcctcatctcttgtgcttgcaatggggtttgctgccttcctgttctgggccatgtccaatggatcttgcagatgcaacatttgtatataa

SEQ ID NO: 76 - DNA, 1683 bp, Unknown
  Deoptimized Influenza A virus
atggagacaattagtctgattactatactattggtcgttacagcgtcaaacgctgacaaaatatgtataggccatcaatccactaattcaaccgaaacagtcgatacactaaccgaaacgaatgtgccagtgacacacgctaaagagctactgcataccgaacataacggaatgctatgcgctactagcctagggcatccactgatactcgatacatgtactatcgagggactcgtatacggtaatcctagttgcgatctactgttaggcggtagggaatggtcatacatagtcgaacgatcatccgccgtaaacggaacatgttatcccggtaatgtcgagaatctcgaagagcttaggacactattctcatccgctagctcataccaacgaatacagatttttcccgatactacatggaatgtgacatataccggaactagtagggcatgttccggatcattctatagatcaatgagatggttgacacaaaaatccggcttttaccctgtgcaagacgcacaatatacgaataataggggtaaatctatactattcgtatggggtatacatcatccacctacttataccgaacagactaatctgtatattagaaacgatacaactacatccgttacaaccgaagacttgaataggacattcaaacccgtaatcggacctagaccactagtgaacggattgcagggtagaatcgattactattggtccgtacttaagccagggcaaacacttagagtgagatctaacggtaatctaatcgcaccatggtacggacacgtacttagcggagggtcacacggtaggatacttaagaccgatctgaaaggggggaattgcgtagtgcaatgccaaaccgaaaaaggcggactgaattcgacactaccattccataatattagcaaatacgcattcggaacatgtcctaagtacgttagggtgaatagtctgaaactcgcagtgggattgagaaacgtacccgctagatcgagtagggggctattcggcgcaatcgcagggtttatcgaaggcggatggccaggactagttgccggatggtacggattccaacatagtaacgatcaaggcgtagggatggccgccgatagggatagcacacaaaaagcaatcgataagattactagtaaggttaataatatagtcgataagatgaataagcaatacgaaattatcgatcacgaatttagcgaagtcgaaactagactgaatatgataaataataagatagacgatcagatacaagacgtatgggcatataacgccgaactgttagtgttgcttgagaatcagaagacactcgacgaacacgacgcaaacgttaataatctgtataataaagtgaaaagagcactagggtctaacgctatggaggacggtaagggatgtttcgaactatatcataaatgcgacgatcaatgcatggagacaattagaaacggtacatataatcggagaaagtatagagaggaatctagactcgaaagacagaaaatcgaaggcgttaaactcgaatccgaaggaacatataagatactgactatttatagtacagtcgctagctcactagtgcttgctatgggattcgccgcattcttgttttgggctatgtcaaacggatcatgtaggtgtaatatttgtatttaa

SEQ ID NO: 77 - DNA, 1497 bp, Influenza A virus
atggcgtcgcaaggcaccaaacgatcctatgaacagatggaaactggtggagaacgccagaatgccactgagatcagggcatctgttggaagaatggttggtggaattgggaggttttacgtacagatgtgcactgaactcaaactcagcgaccaagaaggaaggttgatccagaacagtataacaatagagagaatggttctctccgcatttgatgaaaggaggaacaggtacctagaggaacatcccagtgcggggaaggacccgaagaagaccggaggtccaatctaccgaaggagagacgggaaatgggtgagagagctgattctgtatgacaaagaggagataaggagaatttggcgtcaagcgaacaatggagaagacgcaactgctggtctcactcatatgatgatctggcattccaacctaaatgatgccacataccagagaacaagagccctcgtgcggactggaatggaccccagaatgtgctctctgatgcaaggatcaaccctcccgaggagatctggagctgctggtgcagcaataaagggagtcgggacaatggtaatggaactaattcggatgataaagcgaggcattaatgaccggaacttctggagaggcgataatggacgaagaacaaggattgcatatgagagaatgtgcaacatcctcaaagggaaatttcaaacagcagcacaaagagcaatgatggatcaggtgcgagaaagcagaaatcctgggaatgctgaaattgaagatctcatctttctggcacggtctgcactcatcctgagaggatccgtagcccataagtcctgcttgcctgcttgtgtgtacgggctcgctgtggccagtggatatgattttgagagggaagggtactctctggttgggatagatcctttccgtctgcttcagaacagtcaggtcttcagtcttattagaccaaatgagaatccagcacataaaagtcaattggtatggatggcatgccattctgcagcatttgaggacctgagagtctcaagtttcattagaggaacaagagtgatcccaagaggacaactatccactagaggagttcagattgcttcaaatgagaacgtggaagcaatggattccagcactcttgaactgagaagcagatattgggctataaggaccaggagtggaggaaacaccaatcaacagagagcatctgcaggacaaatcagtgtacagcccactttctcagtacagagaaatcttcccttcgaaagaccgaccattatggctgcgtttaaggggaataccgagggcagaacatctgacatgaggactgaaatcataaggatgatggaaagtgccagaccagaagatgtgtctttccaggggcggggagtcttcgagctctcggacgaaaaggcaacgaacccgatcgtgccttcctttgacatgagtaatgaaggatcttatttcttcggagacaatgcagaggaatatgacaattga

SEQ ID NO: 78 - DNA, 1497 bp, Unknown
  Deoptimized Influenza A virus
atggctagtcagggaacaaaacggtcttacgaacagatggagacaggcggagagagacagaacgcaaccgagattagggctagtgtcggaagaatggtcggagggatcggacgattttacgttcagatgtgtaccgaactcaaactctctgaccaagagggaagactgatacagaattcgattactatcgaaagaatggtgctatccgcattcgacgaacgtaggaataggtatctcgaagagcatcctagcgcaggtaaggatccgaaaaaaaccggagggccaatctatagacgtagagacggtaagtgggttagggaactgatactgtatgacaaagaggagattagaaggatttggagacaggcgaataacggagaggacgcaaccgccggactgacacatatgatgatatggcatagtaatcttaacgacgctacatatcaacggactagggcactcgttagaaccggaatggaccctagaatgtgcagtctgatgcaggggtcaacactccctagaagatccggagccgcaggcgcagcaattaagggagtgggaactatggttatggaactgattagaatgattaagagagggattaacgataggaatttttggcgaggcgataacggaagacgaactagaatcgcatacgaaaggatgtgcaatatccttaagggtaagtttcagactgccgcacaacgagcaatgatggaccaagtgagagagtctagaaatcccggtaacgctgaaatcgaagacctaatctttctcgcacgatccgcactgatacttaggggatccgttgcgcataagtcttgcctacccgcatgcgtatacggactcgcagtcgctagcggatacgatttcgaaagagagggatatagtctcgtagggatcgatccgtttagactgttgcagaatagtcaggtgtttagtctgataagaccgaacgagaatcccgcacataagtctcaactcgtatggatggcatgccattccgccgcattcgaagaccttagggtgagttcgttcattagggggactagagtgatacctagggggcaattgtctactaggggagtgcaaatcgctagtaacgagaatgtcgaagcgatggactctagtacactcgaattgaggtctagatattgggcaatacggactagatccggagggaatacgaatcagcaacgcgctagcgccggacagattagtgtgcaaccaacattctcagtgcaacggaatctcccattcgaaagaccaactattatggccgcattcaaagggaataccgagggacggacatccgatatgagaaccgaaatcataagaatgatggaatccgctagacccgaagacgtaagctttcagggtaggggggtattcgaactatctgacgaaaaagcgactaatccaatcgtaccgtcattcgatatgtctaacgaagggtcatattttttcggcgataacgctgaagagtacgataattga

SEQ ID NO: 79 - DNA, 1404 bp, Influenza A virus
atgaatccaaatcaaaagataatagcacttggctctgtttctataactattgcgacaatatgtttactcatgcagattgccatcttagcaacgactatgacactacatttcaatgaatgtaccaacccatcgaacaatcaagcagtgccatgtgaaccaatcataatagaaaggaacataacagagatagtgcatttgaataatactaccatagagaaggaaagttgtcctaaagtagcagaatacaagaattggtcaaaaccgcaatgtcaaattacagggttcgcccctttctccaaggacaactcaattaggctttctgcaggcggggatatttgggtgacaagagaaccttatgtatcgtgcggtcttggtaaatgttaccaatttgcacttgggcagggaaccactttgaacaacaaacactcaaatggcacaatacatgataggagtccccatagaacccttttaatgaacgagttgggtgttccatttcatttgggaaccaaacaagtgtgcatagcatggtccagctcaagctgccatgatgggaaggcatggttacatgtttgtgtcactggggatgatagaaatgcgactgctagcatcatttatgatgggatgcttaccgacagtattggttcatggtctaagaacatcctcagaactcaggagtcagaatgcgtttgcatcaatggaacttgtacagtagtaatgactgatggaagtgcatcaggaagggctgatactaaaatactattcattagagaagggaaaattgtccacattggtccactgtcaggaagtgctcagcatgtggaggaatgctcctgttacccccggtatccagaagttagatgtgtttgcagagacaattggaagggctccaatagacccgtgctatatataaatgtggcagattatagtgttgattctagttatgtgtgctcaggacttgttggcgacacaccaagaaatgacgatagctccagcagcagtaactgcagggatcctaataacgagagagggggcccaggagtgaaagggtgggcctttgacaatggaaatgatgtttggatgggacgaacaatcaagaaagattcgcgctctggttatgagactttcagggtcgttggtggttggactacggctaattccaagtcacaaataaataggcaagtcatagttgacagtgataactggtctgggtattctggtatattctctgttgaaggaaaaacctgcatcaacaggtgtttttatgtggagttgataagagggagaccacaggagaccagagtatggtggacttcaaatagcatcattgtattttgtggaacttcaggtacctatggaacaggctcatggcctgatggagcgaatatcaatttcatgtctatataa

SEQ ID NO: 80 - DNA, 1404 bp, Unknown
  Deoptimized Influenza A virus
atgaatccgaatcagaaaataatcgcattagggtccgtttcgattactatagcgactatatgcctattgatgcaaatcgcaatactcgcaacgactatgacattgcattttaacgaatgcactaatccctctaataatcaggccgttccatgcgaaccaatcataatcgaacggaatattaccgagatagtgcatcttaacaatacgactatcgaaaaagagtcatgccctaaggtagcggaatataaaaattggtctaagcctcaatgtcagattaccggattcgcaccattctctaaagataattcaattaggcttagcgcaggcggagatatatgggtgactagagagccatacgtaagttgcggactcggtaagtgttatcaattcgcattaggccaagggacaacccttaataataagcatagtaacggtactatacacgataggagtccacataggactcttcttatgaacgagttaggcgtaccattccatttagggactaaacaggtttgtatcgcatggtctagtagttcatgtcatgacggtaaggcatggttgcatgtttgcgttaccggcgacgatagaaacgctaccgcttcaatcatatacgacggtatgcttaccgattcaatcggatcatggtctaaaaatatacttagaacccaagagtccgaatgcgtatgtattaacggtacatgtacagtcgttatgacagacggatccgctagcggtagggccgatacaaagatactattcatacgcgaaggtaagatagtgcatatcggaccattgtccggatccgcacaacacgttgaggaatgctcatgttatcctagatatcccgaagtgagatgcgtatgtagagataattggaaagggtcaaatagacccgtactgtatataaacgttgccgattatagcgtcgatagttcatatgtgtgtagcggactagtgggcgatacacctagaaacgacgattcatctagtagttcgaattgtagggatcctaataacgaaagaggcggaccaggcgttaaagggtgggcattcgataacggtaacgacgtttggatggggagaactattaaaaaagattctagatcagggtatgagacattcagagtggtgggggggtggactaccgctaactctaagtctcaaattaatagacaggtgatagtcgatagcgataattggtcagggtattccggtatttttagcgttgagggtaagacatgtattaataggtgtttttatgtcgaattgattagggggcgaccacaagagactagggtttggtggactagtaattcgattatagtgttttgcggaactagcggaacatacggaaccggatcatggccagacggagcgaatataaattttatgtctatataa

SEQ ID NO: 81 - DNA, 2341 bp, Influenza virus
  CDS (25)..(2298)
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaaaaaagaagaaatggggatcacaactcattttcagagaaagagacgggtgagagacaatatgactaagaaaatgataacacagagaacaataggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgacaagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 82 - protein, 757 aa, Influenza virus
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMKKEEMGITTHFQRKRRVRDNMTKKMITQRTIGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 83 - DNA, 2341 bp, unknown
  synthetic
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggacgttatggagtctatgaaaaaagaggaaatggggattacgacacattttcaacgaaaaagacgggttagggataatatgacaaaaaaaatgattacgcaacgaacaatcggaaagaaaaaacagagactgaataagcgatcatacttgattagggcacttacacttaacactatgactaaggacgccgaaaggggaaagctaaagcgtagagcaattgcaacacccggaatgcaaattagggggttcgtatacttcgtcgagacactcgctagatccatatgcgaaaagttagagcaatccggactgccagtcggggggaacgaaaaaaaagcgaaactcgctaacgtcgttagaaaaatgatgactaatagtcaggataccgaactgtcatttacgattaccggcgataatactaagtggaacgagaatcagaatcctagaatgtttctcgcaatgatcacatatatgacacgtaaccaacccgaatggtttagaaacgtactgtcaatcgcaccaattatgtttagcaataagatggctagattgggcaaggggtatatgtttgaatctaagagtatgaaattgcgaacacagatacctgccgaaatgctagcatcaatcgatctaaagtactttaacgatagtacacgaaaaaaaatcgaaaagattagaccgttactgatagagggaaccgccagcctatcccccggaatgatgatggggatgtttaatatgcttagtaccgtgttaggcgttagcatacttaacttagggcaaaaacgttatactaagactacatattggtgggacggactgcaatctagcgacgatttcgcactaatcgttaacgcacctaaccatgaggggatacaagccggagtcgatagattctatagaacatgcaaactgttagggattaatatgtctaaaaaaaagtcatacataaatagaaccggaacatttgaattcactagctttttttacagatacggattcgttgctaattttagtatggagttacctagtttcggagttagcggaattaacgaatccgccgatatgtcaatcggcgtaaccgttattaagaataatatgattaataacgatctagggccagcaaccgcacaaatggcattgcagttgttcataaaggattatcgttatacatatagatgtcatagaggcgatacacagatacagactagacgatcatttgaaatcaaaaaattgtgggagcaaactaggtctaaagccggactgttagtgtccgacggagggcctaatctatacaatattaggaatctgcatatacccgaagtgtgtctaaagtgggagcttatggacgaagactatcaggggagattgtgcaatccgcttaacccattcgttagccataaagagatagagtcaatgaataacgccgttatgatgccagcacacggacccgctaagaatatggaatacgacgcagtcgcaactacacatagttggataccgaaacggaatcgatccatactgaatacatcccaaagaggcgtactcgaagacgaacaaatgtaccaacggtgttgcaatctatttgaaaaattttttcctagtagtagctatagacgaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 84 - protein, 757 aa, unknown; identical to SEQ ID NO: 82
  Synthetic Construct

SEQ ID NO: 85 - DNA, 2341 bp, unknown
  synthetic
agcgaaagcaggcaaaccatttgaatggatgtcaatccgaccttacttttcttaaaagtgccagcacaaaatgctataagcacaactttcccttatactggagaccctccttacagccatgggacaggaacaggatacaccatggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaagcaatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggacgttatggagtctatgaaaaaagaggaaatggggattacgacacattttcaacgaaaaagacgggttagggataatatgacaaaaaaaatgattacgcaacgaacaatcggaaagaaaaaacagagactgaataagcgatcatacttgattagggcacttacacttaacactatgactaaggacgccgaaaggggaaagctaaagcgtagagcaattgcaacacccggaatgcaaattagggggttcgtatacttcgtcgagacactcgctagatccatatgcgaaaagttagagcaatccggactgccagtcggggggaacgaaaaaaaagcgaaactcgctaacgtcgttagaaaaatgatgactaatagtcaggataccgaactgtcatttacgattaccggcgataatactaagtggaacgagaatcagaatcctagaatgtttctcgcaatgatcacatatatgacacgtaaccaacccgaatggtttagaaacgtactgtcaatcgcaccaattatgtttagcaataagatggctagattgggcaaggggtatatgtttgaatctaagagtatgaaattgcgaacacagatacctgccgaaatgctagcatcaatcgatctaaagtactttaacgatagtacacgaaaaaaaatcgaaaagattagaccgttactgatagagggaaccgccagcctatcccccggaatgatgatggggatgtttaatatgcttagtaccgtgttaggcgttagcatacttaacttagggcaaaaacgttatactaagactacatattggtgggacggactgcaatctagcgacgatttcgcactaatcgttaacgcacctaaccatgaggggatacaagccggagtcgatagattctatagaacatgcaaactgttagggattaatatgtctaaaaaaaagtcatacataaatagaaccggaacatttgaattcacaagttttttctatcgttatgggtttgttgccaatttcagcatggagctccccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatagaggtgacacacaaatacaaacccgaagatcatttgaaataaagaaactgtgggagcaaacccgttccaaagctggactgctggtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaagatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 86 - protein, 757 aa, unknown; identical to SEQ ID NO: 82
  Synthetic Construct

SEQ ID NO: 87 - DNA, 2341 bp, Influenza virus
agcgaaagcaggtcaattatattcaatatggaaagaataaaagaactaagaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgcaggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcagcgattgaatcctatgcatcaacttttaagacattttcagaaggatgcgaaagtgctttttcaaaattggggagttgaacctatcgacaatgtgatgggaatgattgggatattgccagacatgactccaagcatcgagatgtcaatgagaggagtgagaatcagcaaaatgggtgtagatgagtactccagcacggagagggtagtggtgagcattgaccgttttttgagaatccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 88 - DNA, 2341 bp, unknown
  synthetic
agcgaaagcaggtcaattatattcaatatggagagaatcaaagagcttaggaatcttatgtcacaatctagaactagagagatactgactaagactacagtcgatcatatggctataatcaaaaaatatactagcggaagacaggaaaaaaatcccgcacttagaatgaaatggatgatggctatgaaataccctattacagccgataagcgaattaccgaaatgataccagagagaaacgaacagggacagacattgtggtctaaaatgaacgacgccggatccgatagagtgatggtttcgccactagccgtaacatggtggaatagaaacggacctattacgaatacagtgcattaccctaagatatacaaaacatatttcgaaagagtcgagagactgaaacacggaacattcggaccagtgcattttcggaatcaggttaagattagacgtagagtcgatattaatccagggcatgcagatctctccgctaaagaggcacaagacgttattatggaggtcgtgtttcctaacgaggtcggcgctaggatactgactagcgaatcgcaattgacaattacgaaagagaaaaaagaggaactccaggattgcaaaattagcccacttatggtcgcatatatgctcgaacgcgaattggttagaaagactagattcctaccagtcgcaggcggaacgtctagcgtgtatatcgaagtgttgcatctaacacagggaacatgttgggagcaaatgtatactccaggaggcgaagtgagaaacgacgacgttgatcaatcgctaatcatagccgctaggaatatagtgagaagggcagccgttagcgcagacccacttgcgtcactactcgaaatgtgccatagtacgcaaatcggagggattagaatggtcgatatccttaggcagaatcctacagaggaacaggccgtagacatatgcaaagccgcaatgggattgcgaattagctcatcattctcattcggagggtttacgtttaaacggactagcggatctagcgtaaaacgcgaagaggaagtgcttactggcaatctgcaaacactaaagattagggtgcatgagggatacgaagagtttacaatggtcggacgtagagcaaccgctatacttagaaaagcgactaggagactgatacaattgatcgttagcggaagggacgaacagtcaatcgccgaagcgataatagtcgcaatggtgttttcgcaagaggattgcatgattaaggccgttaggggggatctgaatttcgttaatagggctaatcagagactgaatcctatgcatcaattgcttagacattttcagaaagacgctaaagtgttgtttcagaattggggagtcgaacctatcgataacgttatgggtatgatagggatactgccagatatgacaccatcaatcgaaatgtcaatgagaggcgttaggattagtaagatgggcgtagacgaatactccagcactgagagagtggtagtgtcaatcgatagatttcttaggattagggatcagagaggcaacgtactgctatcacccgaagaagttagcgaaacacagggaaccgaaaaattgacaattacgtatagtagtagtatgatgtgggagattaacggaccagagtcagtgttagtgaatacatatcaatggataatacggaattgggagacagtgaaaatacaatggtcacagaatcctacaatgctatacaataagatggagttcgaaccttttcaatcgttagtgcctaaggccataagaggccaatatagtgggttcgttagaacattgtttcagcaaatgagagacgtactcggaacattcgataccgcacagataattaagctattgccattcgcagccgcaccacctaagcaatctagaatgcaattttctagctttaccgttaacgttaggggatccggaatgcgaatactcgttagggggaatagtccagtgtttaattacaataaggcaactaagagattgacagtgttaggcaaggacgcaggaacattgaccgaagacccagacgagggaaccgctggagtggaatccgcagtgcttagggggtttctgatactcggaaaggaggataagagatacggacctgcactatcgattaacgaactatctaatctcgctaaaggcgaaaaagcgaatgtgttaatcggacagggagacgtagtgttagtgatgaaacggaaacgcgatagctcaatactgacagactcacaaaccgctactaagagaattcggatggcaattaattagtgtcgaatagtttaaaaacgaccttgtttctact

SEQ ID NO: 89 - DNA, 2233 bp, Influenza virus
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgctagcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 90 - DNA, 2233 bp, unknown
  synthetic
agcgaaagcaggtactgatccaaaatggaggatttcgttaggcaatgctttaatccaatgatagtcgagttagccgaaaagactatgaaagagtatggcgaagacctaaagattgagactaataaattcgccgcaatttgcacacaccttgaggtttgctttatgtattccgattttcactttattaacgaacagggagagtcaattatagtcgagttaggcgatccgaacgcattgctaaagcatagatttgaaattatagagggacgcgataggacaatggcatggaccgtagttaattcgatttgcaatacaaccggagccgaaaaaccgaaattcttacccgatctatacgattataaagagaataggtttatcgaaatcggagtgactagacgcgaagtgcatatttattatctcgaaaaagcgaataagattaagtccgaaaagacacacatacacatttttagctttaccggagaggaaatggcaacaaaagccgattatacacttgacgaagagtctagggctaggattaagactagactgtttacaattagacaggaaatggctagtagggggttgtgggatagctttagacaatccgaaagaggcgaagagacaatcgaagagagatttgaaattaccggaacaatgcgaaagcttgccgatcaatccctaccccccaatttctctagccttgagaattttagggcatacgttgacggattcgaacctaacggatatatagagggaaagctatcgcaaatgtctaaagaggttaacgctagaatcgaaccattcctaaagacaacacctagaccacttagactgccaaacggaccaccatgctcacagcgatctaagtttctgcttatggacgcactaaagttgtcaatcgaagacccatcacacgagggagaggggataccattgtacgacgcaattaagtgtatgcgaacatttttcggatggaaagagcctaacgtagtgaaaccacacgaaaaagggattaatccgaattatctgcttagttggaaacaggtgttagccgaattgcaggatatcgaaaacgaagagaaaattccgaaaactaagaatatgaaaaaaactagccaactgaaatgggcacttggcgagaatatggcacccgaaaaagtcgatttcgacgattgcaaagacgtcggcgatctaaagcaatacgatagcgacgaacccgaacttagatcactcgctagttggatacagaacgagttcaataaggcatgcgaattgaccgatagctcatggatagagcttgacgagataggcgaagacgtagcaccaatcgaacacatagcctctatgagacggaattattttacatccgaagtgtcacattgtagggcaacagagtatattatgaaaggggtgtatattaataccgcattgcttaacgctagttgcgccgcaatggacgatttccaactgataccgatgatctcgaagtgtagaacaaaagagggacgtagaaagactaatctgtatgggttcattattaagggaaggtctcatttaaggaacgatacagacgtagtgaatttcgttagtatggagtttagccttaccgatccgagactcgaaccacacaaatgggaaaagtattgcgtactagagataggggatatgttgattagatccgcaatcggacaggtttcgagaccaatgtttttgtacgttaggactaacggaacctcgaagattaaaatgaaatggggaatggagatgcgtagatgcctattgcaatcccttcagcaaatcgaatctatgatagaggccgaatctagcgttaaagagaaagatatgacaaaagagttttttgaaaataagtccgaaacatggccaatcggagagtcaccaaaaggggttgaggaatcctcaatcggaaaagtttgtagaacattgctcgcaaaatccgtattcaatagtctatacgccagcccacaactagagggattctctgctgagtcacgaaaactgttactgatagtgcaagcccttagggataatctcgaacccggaacattcgatctaggggggttgtacgaagcaatcgaagagtgtctgattaacgatccatgggtactgcttaacgctagttggtttaattcgttccttacacacgcactatcttagttgtggcagtgctactatttgctatccatactgtccaaaaaagtaccttgtttctact

SEQ ID NO: 91 - DNA, 1775 bp, Influenza virus
  CDS (33)..(1730)
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatatctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacactccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgagattagaatttcagaaatatgaggaaaaacacccttgtttctact

SEQ ID NO: 92 - protein, 565 aa, Influenza virus
MKANLLVLLSALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNIYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNTPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 93 - DNA, 1775 bp, unknown
  synthetic
agcaaaagcaggggaaaataaaaacaaccaaaatgaaggcaaacctactggtcctgttaagtgcacttgcagctgcagatgcagacacaatatgtataggctaccatgcgaacaattcaaccgacactgttgacacagtactcgagaagaatgtgacagtgacacactctgttaacctgttagaggactcacataacggaaagctatgtaggcttaagggaatcgcaccactgcaattgggcaagtgtaatatagccggatggttgttggggaatcccgaatgcgatccactgttacccgttaggtcatggtcatatatagtcgagacacctaatagcgaaaacggaatttgttatcccggcgattttatcgattacgaagagcttagagagcaattgtctagcgttagttcattcgaaagattcgaaatttttccgaaagagtctagttggccaaatcataatactaacggagtgactgccgcatgctcacacgaaggcaagtctagcttttataggaatctgttgtggttgactgagaaagagggatcatatccgaaactgaaaaactcatacgtgaacaaaaagggaaaggaagtgttagtgttgtgggggatacaccatccaccaaatagtaaagagcaacagaatatatatcagaacgaaaacgcatacgttagcgtcgtaactagtaattataatagaaggtttacacccgaaatcgcagagagaccgaaagttagagaccaagccggaagaatgaattattattggacactactgaaacccggcgatacaattatattcgaagcgaacggaaatctgatcgcaccgatgtatgcattcgcactatctagggggttcggatccggaattattactagtaacgctagtatgcacgaatgtaacacgaagtgtcagactccactaggcgcaattaactctagtctgccatatcagaatatacatcccgtaacaatcggcgaatgcccaaaatacgttagatccgctaagcttagaatggttaccggactgagaaatacaccatcaatccaatctagggggttgttcggagcgatagccggatttatcgaaggggggtggacagggatgatagacggttggtacggatatcatcaccaaaacgaacagggatccggatacgcagccgatcagaaatcgacgcaaaacgctattaacggaattactaataaagtgaataccgtaatcgaaaaaatgaatatccaatttaccgcagtcggaaaggaattcaataagcttgagaaaagaatggagaatctgaataaaaaagtcgacgacggatttctagacatatggacttataacgccgaactgttagtgttgctcgaaaacgaaagaacactagactttcacgactcaaacgttaagaatctatacgaaaaagtgaaatcccaattgaaaaataacgctaaagagatagggaacggatgtttcgagttctatcataaatgcgataacgaatgtatggaatccgttaggaacggaacatacgattatcctaagtatagcgaagagtcaaaactgaatagggagaaagtcgacggagtgaaactcgaatcaatggggatatatcagatactggcaatctatagtacagtcgccagctcactggttcttttggtctccctgggggcaatcagtttctggatgtgttctaatggatctttgcagtgcagaatatgcatctgagattagaatttcagaaatatgaggaaaaacacccttgtttctact

SEQ ID NO: 94 - protein, 565 aa, unknown; identical to SEQ ID NO: 92
  Synthetic Construct

SEQ ID NO: 95 - DNA, 1565 bp, Influenza virus
  CDS (46)..(1542)
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 96 - protein, 498 aa, Influenza virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 97 - DNA, 1565 bp, unknown
  synthetic
agcaaaagcagggtagataatcactcactgagtgacatcaaaatcatggcgtcccaaggcaccaaacggtcttacgaacagatggagactgatggagaacgccagaatgccactgaaatcagagctagcgtcggaaaaatgatagggggaatcggaaggttttacatacaaatgtgtaccgaactcaaattgtccgattacgaagggagattgatccaaaatagtctgacaatcgaaagaatggtgttaagcgcattcgacgaaagacggaataagtatctcgaagagcatcctagcgcaggcaaggatccaaaaaaaaccggagggccaatctataggagagtgaacggaaagtggatgcgcgaactgatactgtacgataaagaggagattagacggatatggcgacaagcgaataacggagacgacgctactgccggactgacacatatgatgatatggcactctaatcttaacgacgctacataccaacggactagggcactcgttagaaccggaatggatcctagaatgtgctcacttatgcagggatctacactccctagacgatccggagccgcaggagcagccgttaagggagtcggaactatggttatggaactcgttagaatgataaaaagggggattaacgataggaatttttggagaggcgaaaacggacgtaaaactagaatcgcatacgaaagaatgtgcaatatactcaaagggaaattccaaaccgcagcgcaaaaagctatgatggatcaagttagggagtctaggaatccaggaaatgccgaattcgaagaccttacatttctcgctcggtccgcactaatccttcgcggatcagtcgcacacaaatcttgcttacccgcatgcgtatacggacctgcagtcgctagcggatacgatttcgaacgcgaagggtatagtctagtaggaattgatccatttagattgctccaaaattcgcaagtgtatagtctgattagacctaacgagaatcctgcacacaaatctcaactcgtatggatggcatgccatagtgccgcattcgaagaccttagagtgctatctttcataaagggaacgaaagtgttgcctaggggaaagctatctactaggggagtgcaaatcgctagtaacgagaatatggagactatggagtctagtacactcgaactgagatctagatattgggctattaggactagatccggagggaatacgaatcagcaacgagctagcgccgggcaaatctcaatccaacctacattttccgtgcaacggaatctgccattcgatcggacaacgattatggccgcattcaatgggaataccgagggacggactagcgatatgagaaccgaaattatcagaatgatggaatccgctagaccagaggacgtttcgtttcaaggacggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 98 - protein, 498 aa, unknown; identical to SEQ ID NO: 96
  Synthetic Construct

SEQ ID NO: 99 - DNA, 1413 bp, Influenza virus
agcgaaagcaggggtttaaaatgaatccaaatcagaaaataacaaccattggatcaatctgtctggtagtcggactaattagcctaatattgcaaatagggaatataatctcaatatggattagccattcaattcaaactggaagtcaaaaccatactggaatatgcaaccaaaacatcattacctataaaaatagcacctgggtaaaggacacaacttcagtgatattaaccggcaattcatctctttgtcccatccgtgggtgggctatatacagcaaagacaatagcataagaattggttccaaaggagacgtttttgtcataagagagccctttatttcatgttctcacttggaatgcaggaccttttttctgacccaaggtgccttactgaatgacaagcattcaaatgggactgttaaggacagaagcccttatagggccttaatgagctgccctgtcggtgaagctccgtccccgtacaattcaagatttgaatcggttgcttggtcagcaagtgcatgtcatgatggcatgggctggctaacaatcggaatttcaggtccagataatggagcagtggctgtattaaaatacaacggcataataactgaaaccataaaaagttggaggaagaaaatattgaggacacaagagtctgaatgtgcctgtgtaaatggttcatgttttactataatgactgatggcccgagtgatgggctggcctcgtacaaaattttcaagatcgaaaaggggaaggttactaaatcaatagagttgaatgcacctaattctcactatgaggaatgttcctgttaccctgataccggcaaagtgatgtgtgtgtgcagagacaactggcatggttcgaaccggccatgggtgtctttcgatcaaaacctggattatcaaataggatacatctgcagtggggttttcggtgacaacccgcgtcccgaagatggaacaggcagctgtggtccagtgtatgttgatggagcaaacggagtaaagggattttcatataggtatggtaatggtgtttggataggaaggaccaaaagtcacagttccagacatgggtttgagatgatttgggatcctaatggatggacagagactgatagtaagttctctgttaggcaagatgttgtggcaatgactgattggtcagggtatagcggaagtttcgttcaacatcctgagctaacagggctagactgtatgaggccgtgcttctgggttgaattaatcaggggacgacctaaagaaaaaacaatctggactagtgcgagcagcatttctttttgtggcgtgaatagtgatactgtagattggtcttggccagacggtgctgagttgccattcagcattgacaagtagtctgttcaaaaaactccttgtttctact

SEQ ID NO: 100 - DNA, 1413 bp, unknown
  synthetic
agcgaaagcaggggtttaaaatgaatccaaatcagaaaataacaaccattggatcaatctgtctggtagtcggactaattagcctaatattgcaaatagggaatataatctcaatatggatttcgcattcaatccaaaccggatcacaaaatcatacaggcatatgcaatcagaatataattacttataaaaatagtacatgggtgaaagatactactagcgtgatactaaccggcaattctagtctatgtccgattagggggtgggctatatactctaaagacaatagtatacggatagggtctaagggagacgttttcgtaattagggaaccgtttataagttgttcacatctagagtgtaggaccttttttctgacacaaggcgcactattaaacgataagcattctaacggtacagttaaggataggtcaccttatagggcacttatgtcatgtcccgtaggcgaagcccctagtccatacaatagtagatttgaatccgttgcatggtccgctagcgcatgtcacgacggaatggggtggttgactatagggattagcggacccgataacggagccgttgccgtactgaaatataacggtataattaccgaaactattaagagttggcgtaaaaaaatattgcgtacacaagagtccgaatgcgcatgcgttaacggatcatgttttacaattatgactgacggacctagcgacgggttagcgtcatacaaaatttttaaaatcgaaaaaggcaaggttactaagtcaatcgagttaaacgcacctaattcgcattacgaagagtgttcatgttatcccgataccggaaaggttatgtgcgtttgtagggataattggcacggttcgaacagaccttgggtgtcattcgatcaaaatctagactatcaaatcggatatatatgtagcggagtgttcggcgataatcctagaccagaggacggtacaggcagctgtggaccggtttacgttgacggcgctaacggcgttaaggggtttagttatagatacggcaatggcgtatggatcggtaggactaagtcacatagttctagacacggatttgaaatgatatgggatcctaacggatggaccgaaaccgactcgaagtttagcgttaggcaagacgtagtcgctatgaccgattggtccgggtatagcggatcattcgtgcaacatccagagttaaccggattggattgtatgcgaccatgtttttgggttgagttgattagggggagaccgaaagagaaaactatatggactagcgcgagcagcatttctttttgtggcgtgaatagtgatactgtagattggtcttggccagacggtgctgagttgccattcagcattgacaagtagtctgttcaaaaaactccttgtttctact

SEQ ID NO: 101 - DNA, 1026 bp, Influenza virus
agcgaaagcaggtagatattgaaagatgagtcttctaaccgaggtcgaaacgtacgtactctctatcatcccgtcaggccccctcaaagccgagatcgcacagagacttgaagatgtctttgcagggaagaacactgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctctcgctattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctac

SEQ ID NO: 102 - DNA, 890 bp, Influenza virus
agcaaaagcagggtgacaaagacataatggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcagaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttaataataaaaaacacccttgtttctact

SEQ ID NO: 103 - DNA, 890 bp, unknown
  synthetic
agcaaaagcagggtgacaaagacataatggatccaaacactgtgtcaagctttcaggtagattgctttctttggcatgtccgcaaacgagttgcagaccaagaactaggtgatgccccattccttgaccgactgagacgggatcagaaatcccttaggggcaggggatcgaccctaggcctagacatcgaaaccgcaactagggccggaaagcagatcgtggagcgtatactgaaagaggagtccgacgaagcgcttaagatgactatggccagcgtacccgctagtcggtaccttaccgatatgacactcgaagagatgtcacgcgattggtctatgctaatccctaagcagaaagtggccggacctctatgtatacggatggaccaggcgattatggacaaaaacattatccttaaagcgaacttttccgtgatattcgatcgcctagagactctgatactgttgcgtgcattcacagaagagggagcaattgttggcgaaatttcaccattgccttctcttccaggacatactgctgaggatgtcaaaaatgcagttggagtcctcatcgggggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttaataataaaaaacacccttgtttctact